(19) United States
(12) Patent Application Publication
(10) Pub. No.: US 2025/0313824 A1
(43) Pub. Date: Oct. 9, 2025

(54) FUNCTIONALIZED NUCLEIC ACID CONDENSATES AND RELATED CONDENSATE MONOMERS, LAYERED STRUCTURES, COMPOSITIONS METHODS AND SYSTEMS FOR SEPARATION, PURIFICATION, AND/OR DETECTION OF A BIOMOLECULAR TARGET

(71) Applicant: California Institute of Technology, Pasadena, CA (US)

(72) Inventors: Paul W.K. ROTHEMUND, Pasadena, CA (US); Olivia A. ZOU, Pasadena, CA (US); Jaimie M. STEWART, Pasadena, CA (US); Byoung-jin JEON, San Diego, CA (US); Rustem F. ISMAGILOV, Altadena, CA (US)

(21) Appl. No.: 19/014,032

(22) Filed: Jan. 8, 2025

Related U.S. Application Data
(60) Provisional application No. 63/618,474, filed on Jan. 8, 2024.

(51) Int. Cl. C12N 15/10; C12N 15/115

(52) U.S. Cl. CPC C12N 15/1003; C12N 15/115; C12N 2310/16; C12N 2310/3519

(57) ABSTRACT

Provided herein are functionalized nucleic acid condensate monomers and related functionalized set of nucleic acid condensate monomers, functionalized nucleic acid condensate, as well as related composition methods and systems of manufacturing and related for separation, purification and/or detection of a biomolecular target.

### CROSS REFERENCE TO RELATED APPLICATIONS

The present application claims priority to U.S. Provisional Application No. 63/618,474, entitled “Nucleic Acid Condensates for Separation, Purification and Detection” filed on Jan. 8, 2024, with docket number CIT 8943-P2, which is incorporated herein by reference in its entirety.

### STATEMENT OF GOVERNMENT GRANT

This invention was made with government support under Grant No. MCB 2134772 awarded by the National Science Foundation. The government has certain rights in the invention.

### FIELD

The present disclosure generally relates to biochemistry and molecular biology, and more specifically to functionalized nucleic acid condensates and related condensate monomers, layered structures, components, compositions, methods and systems for separation, purification, and/or detection of a biomolecular target, such as a compounds or cells.

### REFERENCE TO SEQUENCE LISTING

Further, the computer readable form of the sequence listing of the ASCII (XML) text file P3104-US-Seq-List-ST26.xml, created on May 23, 2025, with a size of 29,233 bytes, is incorporated herein by reference in its entirety.

### BACKGROUND

In the biochemistry and molecular biology fields, several processes and reactions involve the separation of mixtures of one or more biomolecular targets, such as microbes and/or compounds comprised in the mixture typically as an analyte, together with additional compounds.

In particular, in the above fields several processes and reactions are known which involve separation of mixtures using a mobile phase and a stationary phase used in combination to separate biomolecular targets in a chromatographic approach based on their different affinities for the mobile and stationary phases.

However, despite the advancement of the technology, performing an efficient and effective chromatography separation of mixtures comprising one or more biochemical targets remains challenging, due to requiring the use of complex and expensive procedures, in particular when performing multiplexed separation and/or separation directed to the recovery of a functional target.

### SUMMARY

Provided herein, are functionalized nucleic acid condensate and related condensate monomers, layered structures components, compositions, methods and systems that can be used for separation, purification, and/or detection of a biomolecular target in a chromatographic approach in which the nucleic acid condensate provides a stationary phase used to perform specific and selective, capture and release of biochemical targets from a mixture with simpler and less costly procedures as well as with higher purity, specificity and accuracy compared to existing chromatography based approaches, as will be understood by a skilled person upon reading of the present disclosure.

In particular, described herein is a composition of matter, a class of new affinity-based materials based on nucleic acid condensates that makes affinity-based separation, purification, and detection of high value targets less expensive, multiplexable, faster, and easier.

Each new affinity-based material is based on one or more nucleic acid monomers (a single strand, or multi-stranded noncovalent complex or covalent complex) which are designed and synthesized so that they can condense, under mild conditions, to form a liquid or gel phase that separates readily from a complex bulk mixture.

In particular a nucleic acid monomer configuration comprises complexes with up to twelve double-stranded arms, wherein at least one of the double-stranded arms configured to interact with one another to form the condensate for example through complementary overlaps that mediate the interactions between condensate monomers.

The nucleic acid monomers herein described are configured to functionalized with binding sites for one or more targets. These binding sites can be implemented with small molecule ligands, such as antibodies, aptamers, or any other class of affinity reagent that can be chemically coupled to the nucleic acid monomers.

When a nucleic acid monomer aggregates it forms both: (1) a concentrated liquid-like or gel-like phase that settles to the bottom of a test tube spontaneously or under light centrifugation, and (2) a dilute gas-like phase on top.

Target molecules are partitioned into the nucleic acid condensate phase, and other components of the complex mixture are excluded in the supernatant. The nucleic acid condensate phase can be washed to remove contaminants. Following washing, the target molecules can be released from the nucleic acid condensate phase using traditional release methods, or in the case that the ligands are nucleic acid aptamers, the target molecules can be released using a highly specific nucleic acid displacement reaction, known as a kleptamer reaction. Once released, the target molecules can be easily separated from the nucleic acid condensate.

Multiple affinity-based reagents can be used simultaneously to achieve multiplexed separation of multiple high value targets from a single mixture. This is achieved by designing multiple affinity-based reagents that separate spontaneously or under light centrifugation into layers within a single test tube, as will be understood by a skilled person upon reading of the present disclosure.

According to a first aspect functionalized nucleic acid condensate monomer is described, configured to specifically bind a biomolecular target, and to form in combination with a same or different nucleic acid condensate monomers in aqueous solvent, a functionalized nucleic acid condensate having a distinct nucleic acid condensate density via liquid-liquid phase separation (LLPS), at condensing thermodynamic conditions.

The functionalized nucleic acid condensate monomer comprises n nucleic acid strands with n being an integer selected from 1 to 12, each nucleic acid strands comprising at least two arm domains ranging from 5 to 75 nucleotides and attached to one another optionally through a flexible linker domain ranging from 1 to 2 nucleotides in length

In the functionalized nucleic acid condensate monomer at least one nucleic acid strand of the n nucleic acid strand further comprises an interaction domain ranging from 1 to 15 nucleotides in length attached to a terminus of an arm domain optionally through a flexible linker domain ranging from 1 to 12 nucleotides in length.

In the functionalized nucleic acid condensate monomer at least one nucleic acid strand of the n nucleic acid strand further comprises a targeting domain ranging from 1 to 50 nucleotides in length which is different from the interaction domain, and is attached to or forms part of a nucleotide region of the arm domain located at a distance from a terminus of the arm domain attached attaches another arm domain, the distance equal to or higher than 10 nucleotides.

In the functionalized nucleic acid condensate monomer, each of the interaction domain, targeting domain and optional linker domain has sequences orthogonal to other sequences of the functionalized nucleic acid monomer

In the functionalized nucleic acid condensate monomer, in the aqueous solution the n nucleic acid strands form a nucleic acid structure through complementary binding of the at least two arm domains, the nucleic acid structure having a melting temperature Tmₘ.

In the nucleic acid structure of the functionalized nucleic acid condensate monomer, the at least two arm domains of the n nucleic acid complementarily binds to another arm domain of a same or different strand at to form at least three duplex arm segments ranging from 5 to 75 nucleotides in length.

In the nucleic acid structure of the functionalized nucleic acid condensate monomer, at least three of the at least three duplex arm segments presents at a terminus an interaction domain configured to form intermolecular interactions with at least one interaction domain of another nucleic acid monomer to initiate a liquid-liquid phase separation of the functionalized nucleic acid condensate at the condensing thermodynamic conditions, the number of interaction domains defining a valency of the functionalized nucleic acid condensate monomer.

In the nucleic acid structure of the functionalized nucleic acid condensate monomer, at least one duplex arm segment comprises the targeting domain attached to and presenting a ligand, capable of specifically binding the biomolecular target in the aqueous solution, at a ligand-target binding temperature Tb at least 10° C. lower than Tmₘ,

According to a second aspect a functionalized nucleic acid nanostar structure is described formed by n nucleic acid strands with n being an integer selected from 3 to 12, the nucleic acid strands configured to form a functionalized nucleic acid condensate monomer of the disclosure having a melting temperature Tmₘ in an aqueous solution

In the functionalized nucleic acid nanostar structure, each strand of the n strands comprises a first arm domain ranging from 5 to 75 nucleotide in length, a second arm domain ranging from 5 to 75 nucleotide in length, at least one of an interaction domain ranging from 1 to 15 nucleotide in length, a targeting domain ranging from 10 to 50 nucleotide in length, and optionally one or more linker domains ranging from 1 to 12 nucleotide in length, each domain having a 5′end and a 3′ end.

In each strand of the n strands of the functionalized nucleic acid nanostar structure the 5′end of the second arm domain is attached to the 3′ end of first arm domain, optionally via the linker domain.

In each of at least three nucleic acid strand of the n strands of the functionalized nucleic acid nanostar structure, the 5′ end of one of the interaction domain is attached to the 3′ end of the second arm domain, or in the alternative the 3′ end of the interaction domain is attached to the 5′ of the first arm domain.

In at least one strand of the n strands of the functionalized nucleic acid nanostar structure, the targeting domain is attached to or forms part of one of the first arm domain or the second arm domain, at a distance from a terminus of the one of the first arm domain or the second arm domain attached to the other of the first arm domain and the second arm domain of the at least one strand, In at least one strand, the distance is 10 nucleotides or higher.

In the functionalized nucleic acid nanostar structure, each of the interaction domain, targeting domain and optional linker domain has sequences orthogonal to other sequences of the functionalized nucleic acid nanostructure.

In the functionalized nucleic acid nanostar structure, the first arm domain of each strand complementarily binds the second arm domain of a first another strand of the nanostructure, and the second arm domain of each strand complementarily binds to the first arm domain of a second another strand of the nanostructure at the condensing thermodynamic conditions, to form n duplex arms segments of the functionalized nucleic acid nanostructure, each duplex arm segments independently ranging from 5 to 75 nucleotides in length

In the functionalized nucleic acid nanostar structure, at least three duplex arm segments present the interaction domain is configured to form at the condensing thermodynamic conditions intermolecular interactions with an interaction domain of a different nucleic acid nanostructure, the intermolecular interactions configured to initiate under the condensing thermodynamic conditions the liquid-liquid phase separation of the functionalized nucleic acid condensate,

In the functionalized nucleic acid nanostar structure at least one duplex arm segment comprises the targeting domain attached to and presenting a ligand configured to specifically bind the biomolecular target in the aqueous solution, at a ligand-target binding temperature Tb which is least 10° C. lower than Tmₘ

According to a third aspect a functionalized set of nucleic acid condensate monomers is described, configured to specifically bind a biomolecular target, and to form, in aqueous solvent and under condensing thermodynamic conditions, a functionalized nucleic acid condensate having a distinct nucleic acid condensate density via liquid-liquid phase separation (LLPS).

The functionalized set of nucleic acid condensate monomers comprises one or more functionalized nucleic acid monomers of the disclosure each configured to form a nucleic acid condensate at the condensing thermodynamic conditions through intermolecular interactions of interaction domains of another, same or different nucleic acid condensate monomer of the set of nucleic acid condensate monomers, and presenting at least one ligand configured to specifically bind the biomolecular target at a same temperature Tb.

In preferred embodiments, at least one condensate monomers of the functionalized set of nucleic acid condensate monomers is a functionalized nucleic acid nanostar structure herein described.

According to a fourth aspect a functionalized nucleic acid condensate is described having a distinct functionalized nucleic acid condensate density and configured to bind a biomolecular target. The functionalize nucleic acid condensate comprises a functionalized set of nucleic acid condensate monomers herein described configured to form a condensate in aqueous solvent and under the condensing thermodynamic conditions, the functionalized nucleic acid condensate having the distinct functionalized nucleic acid condensate density, via liquid-liquid phase separation (LLPS).

According to a fifth aspect a functionalized nucleic acid condensate is described, comprising one or more nucleic acid condensate monomers formed by a functionalized nucleic acid nanostar structure of the disclosure. In the functionalized nucleic acid condensate, the functionalized nucleic acid nanostar structure presents an interaction domain configured to form molecular interactions with other monomers of the functionalized nucleic acid condensate, and a ligand configure to bind a biomolecular target and presented on the nanostructure for binding with the target biomolecular target.

According to a sixth aspect a functionalized layered nucleic acid condensate structure, is described comprising: at least one functionalized nucleic acid condensate layer of the disclosure, each condensate layer having a distinct condensate density and comprising a distinct interaction domains and a distinct ligand specific for distinct one or more biomolecular targets.

In the nucleic acid condensate structure, at condensing thermodynamic conditions the at least one functionalized nucleic acid condensate layer is arranged in the structure, in order of increasing density, with the densest layer positioned opposite the least dense layer within the structure. In some embodiments the functionalized layered nucleic acid condensate structure comprises a diffuse layer and the diffuse layer is the least dense layer in the structure.

In the nucleic acid condensate structure, each distinct ligand specific for the one or more biomolecular target is in a configuration in which the ligand is presented for binding to the biomolecular target at a same temperature Tb, the binding facilitating selective capturing of the target compound within the nucleic acid condensate layer containing the ligand.

In preferred embodiment, the distinct interaction domains of each nucleic acid condensate layer are orthogonal to the distinct interaction domains of a different condensate layer of the functionalized layered nucleic acid condensate structure.

According to a seventh aspect a method and a system are described to separate a biomolecular target from a mixture in which the biomolecular target is comprised together with additional compounds. The method comprises providing a set of functionalized nucleic acid condensate monomer herein described functionalized with a ligand specific for the biomolecular target,

The method further comprises contacting the set of functionalized nucleic acid condensate monomers with the mixture to allow binding of the biomolecular target with the ligand, and. following the contacting with the mixture, inducing condensation of the set of functionalized nucleic acid condensate monomers to form a functionalized nucleic acid condensate.

The method also comprises layering of the functionalized nucleic acid condensate in nucleic acid condensate layer within a layered nucleic acid condensate structure of the disclosure; contacting the layered nucleic acid condensate structure with a release agent configured to specifically release the biomolecular target from the ligand of the functionalized nucleic acid monomer to obtain the release of the biomolecular target from the nucleic acid condensate; and separating the released biomolecular target from the nucleic acid condensate layer.

The system to separate a biomolecular target from a mixture in which the biomolecular target is comprised together with additional compounds. comprises at least one functionalized set of nucleic acid condensate monomers herein described functionalized with a ligand specific for the biomolecular target, and one or more release agents capable of releasing the biomolecular target from the functionalized nucleic acid condensate monomer.

According to an eight aspect a method and systems are described to separate at least two biomolecular targets from a mixture optionally comprising additional compounds. The method comprises providing at least two sets of functionalized nucleic acid condensate monomers herein described each set functionalized with one or more ligand specific for one or more of the at least two biomolecular targets, each of the at least two sets of nucleic acid condensate monomers configured to form a nucleic acid condensate layer having a distinct density

The method further comprises contacting the at least two sets of functionalized nucleic acid condensate monomers with the mixture to allow binding of the biomolecular target with the ligand, and. following the contacting, inducing condensation of the at least two sets of functionalized nucleic acid condensate monomers to form at least two nucleic acid condensates one for each set of monomers.

The method also comprises layering of the at least two functionalized nucleic acid condensates in at least two polynucleotide condensate layers within a layered nucleic acid condensate structure of the present disclosure; contacting the layered nucleic condensate structure with at least two release agents each configured to specifically release a biomolecular target from a corresponding ligand of the at least two sets of functionalized nucleic acid monomer to obtain the release of the at least biomolecular target from of the at least two sets of nucleic acid condensate monomers; and separating the released at least two biomolecular target from said the nucleic acid condensate layer.

The system comprises at least two sets of functionalized nucleic acid condensate monomers herein described each functionalized with one or more ligand specific for one or more of the at least two biomolecular targets, and at least two release agents capable of releasing the at least biomolecular targets from the at least two sets of functionalized nucleic acid condensate monomers.

According to a ninth aspect, a method and a system are described to manufacture a functionalized nucleic acid condensate monomer of the present disclosure configured to specifically bind a biomolecular target and to form a functionalized nucleic acid condensate having a distinct density.

The method comprises: providing n nucleic acid strands with n being an integer selected from 1 to 12, each nucleic acid strands comprising at least two arm domains ranging from 5 to 75 nucleotides and attached to one another optionally through a flexible linker domain ranging from 1 to 2 nucleotides in length, wherein
at least one nucleic acid strand of the n nucleic acid strand further comprises an interaction domain ranging from 1 to 15 nucleotides in length attached to a terminus of an arm domain optionally through a flexible linker domain ranging from 1 to 12 nucleotides in length.
at least one nucleic acid strand of the n nucleic acid strand further comprises a targeting domain ranging from 1 to 50 nucleotides in length which is different from the interaction domain, and is attached to or forms part of a nucleotide region of the arm domain located at a distance from a terminus of the arm domain attached attaches another arm domain, the distance equal to or higher than 10 nucleotides, with the provisos that if a duplex arm segment has a length less than 10 nucleotides, then the targeting domain is attached to the terminus of the arm domain that constitutes part of the terminus of the duplex arm segment,
each of the interaction domain, targeting domain and optional linker domain has sequences orthogonal to other sequences of the functionalized nucleic acid monomer

The method further comprises contacting the n nucleic acid strands with an aqueous solution for a time and under conditions to allow formation of a nucleic acid structure configured form in combination with a same or different nucleic acid structure a nucleic acid condensate having a distinct nucleic acid condensate density via liquid-liquid phase separation (LLPS), at condensing thermodynamic conditions.

The method further comprises attaching the ligand to the targeting domain of the n nucleic acid strands before or after the contacting to provide the functionalized nucleic acid condensate monomer of the disclosure

Optionally the method can further comprise inducing condensation of the functionalized set of nucleic acid condensate monomer to form a functionalized nucleic acid condensate, and detecting a density of the functionalized nucleic acid condensate to detect the distinct density of the functionalized nucleic acid condensate.

The system to manufacture a functionalized nucleic acid condensate monomer of the present disclosure comprises a set n nucleic acid strands with n being an integer selected from 1 to 12, in which
each nucleic acid strand comprising at least two arm domains ranging from 5 to 75 nucleotides and attached to one another optionally through a flexible linker domain ranging from 1 to 2 nucleotides in length, wherein
at least one nucleic acid strand of the n nucleic acid strand further comprises an interaction domain ranging from 1 to 15 nucleotides in length attached to a terminus of an arm domain optionally through a flexible linker domain ranging from 1 to 12 nucleotides in length.
at least one nucleic acid strand of the n nucleic acid strand further comprises a targeting domain ranging from 1 to 50 nucleotides in length which is different from the interaction domain, and is attached to or forms part of a nucleotide region of the arm domain located at a distance from a terminus of the arm domain attached attaches another arm domain, the distance equal to or higher than 10 nucleotides,
each of the interaction domain, targeting domain and optional linker domain has sequences orthogonal to other sequences of the functionalized nucleic acid monomer

The system further a ligand configured to specifically bind a biomolecular target and optionally agents for attaching the ligand to the nucleic acid strand configured for attachment to the targeting domain of the n nucleic acid strands according to a method to manufacture a functionalized condensate monomer herein described.

The system can optionally further comprise condensation agents, and devices to detect density of a corresponding nucleic acid condensate as will be understood by a skilled person upon reading of the disclosure.

According to a tenth aspect, a method and a system are described to provide a functionalized nucleic acid condensate herein described. The method comprises providing a set of functionalized nucleic acid condensate monomers herein described configured to form a nucleic acid condensate having a distinctive density; and inducing condensation of the set of functionalized condensate monomer to form a functionalized nucleic acid condensate having the distinctive density.

The system comprises a set of functionalized condensate monomer herein described configured to form a nucleic acid condensate having a same density in combination with one or condensation agents and or devices configured to provide the condensing thermodynamic conditions.

According to a eleventh aspect, a method and a system are described to provide a functionalized nucleic acid condensate layered structure herein described configured to separate a biomolecular target.

The method comprises providing a set of functionalized nucleic acid condensate monomers herein described configured to form a nucleic acid condensate having a distinctive density; the set comprising at least one functionalized nucleic acid condensate monomer attaching a ligand specific for the biomolecular target, in a configuration in which the ligand is presented for binding to the biomolecular target when present, the binding facilitating selective capturing of the target compound within the nucleic acid condensate layer containing the ligand

The method further comprises inducing condensation and layering of the set of functionalized condensate monomer in an aqueous solvent to form a functionalized nucleic acid condensate layer of the disclosure, and a diffuse phase layer, each layer having a distinct density. the diffuse phase layer and the functionalized nucleic acid condensate layer arranged in the structure, in order of increasing density, with the densest layer positioned opposite the least dense layer within the structure the diffuse layer being the least dense layer in the structure.

The system comprises a set of functionalized condensate monomer herein described configured to form a nucleic acid condensate having a same density and configured to specifically bind the biomolecular target. In the system a set of functionalized condensate monomers is comprised in combination with condensation and layering agents

According to twelfth aspect, a method and a system are described to provide a functionalized layered nucleic acid condensate structure herein described configured to separate two or more biomolecular target.

The method comprises providing at least two set of functionalized nucleic acid condensate monomers herein described configured to form a nucleic acid condensate having a distinctive density; each of the at least two set comprising at least functionalized nucleic acid condensate monomer attaching a ligand specific a distinct biomolecular target of the two or more biomolecular targets, in a configuration in which the ligand is presented for binding to the biomolecular target when present, the binding facilitating selective capturing of the target compound within the nucleic acid condensate layer containing the ligand

The method further comprises inducing condensation and layering of the at least two set of functionalized condensate monomer in an aqueous solvent to form at least two functionalized nucleic acid condensate layer of the disclosure, and a diffuse phase layer, each layer having a distinct density. The diffuse phase layer and the at least one functionalized nucleic acid condensate layer arranged in the structure, in order of increasing density, with the densest layer positioned opposite the least dense layer within the structure the diffuse layer being the least dense layer in the structure.

The system comprises at least two set of functionalized condensate monomer herein described configured to form a nucleic acid condensate having a same density and configured to specifically bind a distinct target of the two or more biomolecular target. In the system the at least two sets of functionalized condensate monomers are comprised in combination with condensation and layering agents.

Functionalized nucleic acid condensate and related layered structures, components, compositions, methods and systems herein described can be used in several embodiments to perform separation, purification, and/or detection of one or more biomolecular targets at a lower cost compared to existing affinity based-chromatography. Nucleic acid condensates can be less expensive than other affinity-based reagents. Accordingly the costs of the functionalized nucleic acid condensate and related components, compositions, methods and systems herein described can be one or two orders of magnitude lower per milliliter if compared with the cost of other affinity materials such as antibody-based materials.

Functionalized nucleic acid condensate and related layered structures, components, compositions, layered structures methods and systems herein described can be used in several embodiments to perform separation, purification, and/or detection of one or more biomolecular target with a simplified process compared to existing affinity based-chromatography approaches. In particular, no nanoparticle synthesis and no chemical coupling of the affinity reagent to particles or resin are required in connection with functionalized nucleic acid condensate of the present disclosure, which fact simplifies the related set up and execution as will be understood by a skilled person.

In particular, functionalized nucleic acid condensate and related layered structures, components, compositions, methods and systems herein described can be used in several embodiments to effectively perform multiplexed separation, purification, and/or detection of one or more biomolecular target with simpler process compared to existing affinity columns which are typically used in series to perform multiplexed detection. Functionalized nucleic acid condensate of the present disclosure allow configuration of multiple orthogonal condensates which spontaneously phase separate into at least three layers each layer can separate a different target or set of targets simultaneously, in the same step

Functionalized nucleic acid condensate and related layered structures, components, compositions, methods and systems herein described can be used in several embodiments to effectively perform separation, purification, and/or detection of one or more biomolecular target with an improved recovery compared to existing affinity based-chromatography approaches. In particular functionalized nucleic acid condensate allow recovery of separated biomolecular target with no need to perform harsh chemical step that can denature the target which is later renatured, a step that causes decreases to yield. Functionalized nucleic acid condensate also allow recovery of separated biomolecular target minimizing the release of nonspecifically bound components that are undesired, decreasing the purity of the final product.

Accordingly, in some embodiments, functionalized nucleic acid condensate and related components, compositions, methods and systems herein described can be used in several embodiments to perform separation of a biomolecular target of interest with highest purity (95% or higher) to maximize safety and efficacy of subsequent use of the biomolecular target. Subsequent uses that can benefit from high purity separation comprise therapeutic uses (for biomolecular target which are used as therapeutic agents) biochemical transformation (for biomolecular target which are enzymes), as well as structure determination (for biomolecular targets which is crystallized).

Functionalized nucleic acid condensate and related layered structures, components, compositions, methods and systems herein described can be used in several embodiments to effectively perform detection of one or more biomolecular target improving the resolution of the detection with respect to existing affinity based-chromatography approaches. The absence of particles used as a stationary phase allows easier detection of the separate biomolecular target and because multiple layers can be designed to bind multiple targets, the capture of different targets can be measured during the same separation.

Additionally, functionalized nucleic acid condensate and related layered structures, components, compositions, methods and systems herein described can be used in several embodiments to effectively perform separation, purification, and/or detection of one or more biomolecular target minimizing the presence of other component of the mixture due to the absence of stationary phase formed by particles or resins and to the dense nature of the condensate which tends to exclude molecules that do not have strong specific interactions with the condensate monomers, as will be understood by a skilled person.

Accordingly functionalized nucleic acid condensate and related layered structures, components, compositions, methods and systems herein described can be used in several embodiments to perform separation and subsequent quantitative or qualitative detection of one or more biomolecular targets with high sensitivity, high accuracy, high specificity, and low limit of detection, to maximize the reliability of the subsequent use of the qualitative or quantitative detection provided. Subsequent uses that can benefit from high accuracy, high sensitivity, high specificity, and low limit of detection comprise for example diagnosis or determination of occurrence of biological event following detection of biomolecular targets comprising biomarker. Examples include measurement of troponin which peaks in concentration 3-12 hours after a heart attack, or prostate specific antigen whose elevated level in blood is an indicator of prostate cancer.

The functionalized nucleic acid condensate and related layered structures, components, compositions, methods and systems herein described can be used in connection with various applications wherein separation, purification and/or detection of mixtures comprising one or more biomolecular targets together with other compounds is desired. For example, methods and systems herein described and related compositions can be used in applications to separate, purify detect and/or amplify nucleic acids, proteins, simple compounds, cells, viruses from mixtures such as processed or unprocessed samples. Additional exemplary applications include separation and/or uses of the separated nucleic acid and/or target compounds in several fields including medical applications such as clinical applications and diagnostics, basic biology research, applied biology, bio-engineering, medical research, diagnostics and therapeutics identification and uses, and in additional fields identifiable by a skilled person upon reading of the present disclosure.

The details of one or more embodiments of the disclosure are set forth in the accompanying drawings and the description below. Other features, objects, and advantages will be apparent from the description and drawings, and from the claims.

### BRIEF DESCRIPTION OF DRAWINGS

The accompanying drawings, which are incorporated into and constitute a part of this specification, illustrate one or more embodiments of the present disclosure and, together with the description of example embodiments, serve to explain the principles and implementations of the disclosure.

FIG. 1 Panels A-B shows a schematic illustration of nucleotide condensate monomers configured to form a condensate under condensing thermodynamic conditions and formed by a single strand (FIG. 1 Panel A), or four nucleic acid strands complementarily bound to form a nucleic acid nanostar structure (FIG. 1 Panel B).

FIG. 2 Panel A-B shows a schematic illustration of the nucleotide condensate monomers of FIG. 1 Panels A to B respectively configured to form a condensate under condensing thermodynamic conditions and presenting a ligand binding a biomolecular target. In particular FIG. 2 Panel A shows a single stranded nucleic acid condensate monomer, and FIG. 2 Panel B shows a four stranded nucleic acid condensate monomer forming a nucleic acid nanostructure, each functionalized with a ligand and corresponding target.

FIG. 3 shows results of experiments performed by Biff et al 2013 and shown in FIG. 1 of Biffi et al 2013 [1] including the overall architecture of multi-arm nanostars, a 3-arm and 4 arm example schematics, experiments having capillaries with liquids, and an experimental phase diagram. FIG. 3 Panel A shows a schematic illustration of a DNA nanostar structure and having valence 4 in which each arm terminate with one sticky overhang each. FIG. 3 Panel B show the phase behavior of DNA nanostars with valence f=3 and f=4 formed by the self-assembly of three and four oligomers, respectively from FIGS. 1A and 1B of Biffi et al 2013 [1]. FIG. 3 Panel C shows Fluorescent emission from a capillary tube containing a sample of EtBr-marked f=4 nanostars photographed after the sample was centrifuged from FIG. 1C of Biffi et al 2013 [1]. FIG. 3 Panel D shows experimentally determined consolution curve for nanostars with f=3 (dark gray dots) and f=4 (light gray dots), from FIG. 1E of Biffi et al 2013 [1]

FIG. 4 shows a schematic representation of the biomolecular condensation and its features shown in Alberti et al 2017 [2], Sato et al 2019 [3] and Jeon et al [4] In particular, FIG. 4 Panel A shows an illustration of the related repertoire from FIG. 4 of Alberti et al 2017 [2] FIG. 4 Panel B shows an illustration of the compartments on a microscopic scale formed by the microscopic images of DNA droplets from FIG. 5C and the schematics of FIG. 6B. of Sato et al 2019 [3] and FIG. 4 Panel C shows an illustration of the compartments on a macroscopic scale formed by a Bulk phase separation of 100 μL of NS solution. NSs are visualized by adding YOYO-1 at 1:100 dye: NS molar ratio from FIG. 1A of Jeon et al [4].

FIG. 5 shows a schematic representation of structural features of a condensate monomer affect liquid properties of a condensate, such as density.

FIG. 6 shows schematics chart and picture reporting results of experiments illustrating that stronger sticky ends form denser DNA condensates. FIG. 6 Panel A shows schematics for 5 stars of decreasing sticky end strength. FIG. 6 Panel B is cartoon diagram showing the liquid phase concentration (in say mg/ml) vs strength of the sticky end (free energy, ΔG, e.g. kcal/mol). It shows that the liquid phase concentration should decrease with decreasing sticky end strength. FIG. 6 Panel C shows experimental data for condensates having 5 different sticky end strengths. FIG. 6 Panel D is the experimental analog of FIG. 6 Panel B.

FIG. 7 shows schematic representation and illustration of an exemplary separation of nanostars into 2 layers based on the number of arms, holding other structural properties roughly constant, using 4-armed and 6-armed nanostars. FIG. 7 Panel A shows schematic of the two nanostars used, 4-armed nanostar NS-A and a 6-armed nanostar NS-B. FIG. 7 Panel B and FIG. 7 Panel C shows Data showing the clear phase separation of nanostars based on the number of arms.

FIG. 8 shows a schematic representation and illustration reporting an exemplary separation performed in accordance with the disclosure. FIG. 8 Panel A is a schematic cartoon showing the separation of condensate layers based on the valency of the nanostars (number of sticky ends) which here corresponds with the number of arms. FIG. 8 Panel B shows separation of nanostars into 3 layers based on the strength of sticky ends, holding other structural properties roughly constant; ΔΔG=1.4 kcal/mol is demonstrated, meaning that no more than ΔΔG=1.4 to differentiate layers.

FIG. 9 shows schematics and illustrations reporting an exemplary separation of nanostars into 3 layers based on the average number of arms, holding other structural properties roughly constant, using 4-armed stars (NS_{A}), a 50-50 mix of 4 and 8-armed nanostars (NS_{b}), and 8-armed nanostars (NS_{c}).

FIG. 10 shows schematics and illustrations reporting an exemplary separation of nanostars into 3 layers based on the average number of arms, using a constant lower temperature nanostar formation protocol, and shorter, higher speed centrifugation.

FIG. 11 shows schematics illustrating the analogy between the use of a classic separatory funnel and the use of layered DNA condensates.

FIG. 12 shows schematics and illustrations exemplifying the basic workflow for the separation of molecules using a layered DNA condensate.

FIG. 13 schematics and illustrations reporting results demonstrating the capturing streptavidin in a DNA condensate via an Aptamer

FIG. 14 shows schematics and illustrations reporting an exemplary demonstration of how the salt dependence of streptavidin capture in a liquid layer is measured.

FIG. 15 shows a chart, schematics and illustrations reporting an exemplary embodiment in which streptavidin capture by aptamers in a DNA condensate saturates at 750 mM Sodium.

FIG. 16 shows schematics and illustrations showing an exemplary embodiment in which the addition of a release strand frees streptavidin into the gas phase.

FIG. 17 shows schematics and illustrations showing experimental condensates that demonstrate the principles required for separating targets with layered condensates.

FIG. 18 shows the use of nucleic acid condensates to separate and quantify bacteria, such as those responsible for bacterial sepsis. In particular, FIG. 18 shows artificial DNA (FIG. 18a-d) and RNA (FIG. 18h) condensates which will be fused to appropriate aptamers and used to capture bacteria nonspecifically (e) and specifically (j). Liquids will be separated centrifugally (FIG. 18d, FIG. 18f, FIG. 18j) or via wetting (FIG. 18i, FIG. 18k) and bacteria will be counted by culture (FIG. 18g) or digital LAMP (FIG. 18l, [5]).

FIGS. 19A and 19B show schematic representation of three different condensates that bind three different targets. FIG. 19A describes a condensate that has a ligand for the HIV-1 TAT peptide (labeled yellow, with FAM as the fluorophore). FIG. 19B describes a condensate that has a ligand for peptide P22 (labeled blue, with AF647 as the fluorophore). FIG. 19C describes a condensate that has a ligand for streptavidin (labeled red, with AF555 as the fluorophore. Colors are false colors and do not correspond to the normal fluorescence colors of the emitters.

FIG. 20 (SEQ ID 15 to SEQ ID 30) reports the sequences for the RNA condensates described in FIGS. 19A and 19B and experimentally imaged in FIG. 21.

FIG. 21 shows selected micrograph fields of the 3 condensates described in FIGS. 19A-C which show that at a microscopic level, three different condensates with three different ligands can compartmentalize three different targets, each one labeled with a different fluorescent color (red, yellow, or blue). The RNA condensates themselves are colorless, and would have no color without the fluorescently labeled targets. A mix of targets was applied to the condensates and the targets were spontaneously compartmentalized in the appropriate condensates.

FIG. 22 shows data illustrating exemplary features of an exemplary a 4 layer condensate built using the principle of varying only sticky end strength, where stronger sticky ends have greater density. FIG. 22 Panel A (SEQ ID 31 to SEQ ID 34) gives nanostar names, sticky end energies, and the Hamming distances and slide distances between adjacent condensates. These were minimized, with a target of [6, 0] (6 Hamming distance and 0 slide distance) for the purpose of keeping adjacent layers orthogonal and immiscible.) FIG. 22 Panel B shows the results of annealing only at left (which is insufficient for correct ordering) and the results of centrifuging after annealing which gives the correct layer ordering.

FIG. 23 shows data illustrating exemplary features of an exemplary a 5 layer condensate built on the principle of changing more than one variable, as long as the two changes in structural feature agree on the direction of the change of the trend in density. FIG. 23 Panel A documents the changed variables. FIG. 23 Panel B gives nanostar names, sticky end energies, and the Hamming distances and slide distances between adjacent condensates. These were minimized, with a target of [6, 0] (6 Hamming distance and 0 slide distance) for the purpose of keeping adjacent layers orthogonal and immiscible.) FIG. 23 Panel C gives the results, which shows that all nanostar condensates layer in order correctly.

### DETAILED DESCRIPTION

Provided herein, are a functionalized nucleic acid condensate and related functionalized nucleic acid monomers, layered structures components, compositions, methods and systems that can be used for separation, purification, and/or detection of a biomolecular target.

The terms “nucleic acid” “NA” or “polynucleotide” as used herein indicates an organic polymer composed of two or more monomers including nucleotides, nucleosides or analogs thereof. The term “nucleotide” refers to any of several compounds that consist of a ribose or deoxyribose sugar joined to a purine or pyrimidine base and to a phosphate group and that is the basic structural unit of nucleic acids. The term “nucleoside” refers to a compound (such as guanosine or adenosine) that consists of a purine or pyrimidine base combined with deoxyribose or ribose and is found especially in nucleic acids. The term “nucleotide analog” or “nucleoside analog” refers respectively to a nucleotide or nucleoside in which one or more individual atoms have been replaced with a different atom or a with a different functional group. Exemplary functional groups that can be comprised in an analog include methyl groups and hydroxyl groups and additional groups identifiable by a skilled person.

Exemplary monomers of a polynucleotide comprise deoxyribonucleotide, ribonucleotides, LNA nucleotides and PNA nucleotides. The term “deoxyribonucleotide” refers to the monomer, or single unit, of DNA, or deoxyribonucleic acid. Each deoxyribonucleotide comprises three parts: a nitrogenous base, a deoxyribose sugar, and one or more phosphate groups. The nitrogenous base is typically bonded to the 1′ carbon of the deoxyribose, which is distinguished from ribose by the presence of a proton on the 2′ carbon rather than an —OH group. The phosphate group is typically bound to the 5′ carbon of the sugar.

The term “DNA” or “deoxyribonucleic acid” as used herein indicates a polynucleotide composed of deoxyribonucleotide bases or an analog thereof to form an organic polymer. The term “deoxyribonucleotide” refers to any compounds that consist of a deoxyribose (deoxyribonucleotide) sugar joined to a purine or pyrimidine base and to a phosphate group, and that are the basic structural units of a deoxyribonucleic acid, typically adenine (A), cytosine (C), guanine (G), and thymine (T). In an DNA adjacent ribose nucleotide bases are chemically attached to one another in a chain typically via phosphodiester bonds. The term “deoxyribonucleotide analog” refers to a deoxyribonucleotide in which one or more individual atoms have been replaced with a different atom with a different functional group. For example, deoxyribonucleotide analogues include chemically modified deoxyribonucleotides, such as methylation hydroxymethylation glycosylation and additional modifications identifiable by a skilled person.

The term “modified nucleotides” refers to a nucleic acid monomer that is not the standard DNA or RNA nucleotide or nucleoside. In particular, modified nucleotides comprise nucleotide analogs presenting one or more individual atoms which have been replaced with a different atom or with a different functional group. Exemplary functional groups that can be comprised in an analog include methyl groups and hydroxyl groups and additional groups identifiable by a skilled person.

The term “ribonucleotide” refers to the monomer, or single unit, of RNA, or ribonucleic acid. Ribonucleotides have one, two, or three phosphate groups attached to the ribose sugar. The term “RNA” or “ribonucleic acid” as used herein indicates a polynucleotide composed of ribonucleotide bases or an analog thereof linked to form an organic polymer. The term “ribonucleotide” refers to any compounds that consist of a ribose (ribonucleotide) sugar joined to a purine or pyrimidine base and to a phosphate group, and that are the basic structural units of a ribonucleic acid, typically adenine (A), cytosine (C), guanine (G), and uracil (U). In an RNA adjacent ribose nucleotide bases are chemically attached to one another in a chain typically via phosphodiester bonds.

The term “locked nucleic acids” (LNA) as used herein indicates a modified RNA nucleotide. The ribose moiety of an LNA nucleotide is modified with an extra bridge connecting the 2′ and 4′ carbons. The bridge “locks” the ribose in the 3′-endo structural conformation, which is often found in the A-form of DNA or RNA. LNA nucleotides can be mixed with DNA or RNA bases in the oligonucleotide whenever desired. The locked ribose conformation enhances base stacking and backbone pre-organization. This significantly increases the thermal stability (melting temperature) of oligonucleotides. LNA oligonucleotides display unprecedented hybridization affinity toward complementary single-stranded RNA and complementary single- or double-stranded DNA. Structural studies have shown that LNA oligonucleotides induce A-type (RNA-like) duplex conformations as will be understood by a skilled person.

The term “polyamide polynucleotide”, “peptide nucleic acid” or “PNA” as used herein indicates a type of artificially synthesized polymer composed of monomers linked to form a backbone composed of repeating N-(2-aminoethyl)-glycine units linked by peptide bonds. The various purine and pyrimidine bases are linked to the backbone by methylene carbonyl bonds. Since the backbone of PNA contains no charged phosphate groups, the binding between PNA/DNA strands is stronger than between DNA/DNA strands due to the lack of electrostatic repulsion. PNA oligomers also show greater specificity in binding to complementary DNAs, with a PNA/DNA base mismatch being more destabilizing than a similar mismatch in a DNA/DNA duplex. This binding strength and specificity also applies to PNA/RNA duplexes. PNAs are not easily recognized by either nucleases or proteases, making them resistant to enzyme degradation. PNAs are also stable over a wide pH range. In some embodiments, polynucleotides can comprise one or more non-nucleotidic or non nucleosidic monomers identifiable by a skilled person.

Accordingly, the term “polynucleotide” includes nucleic acids of any length, and in particular DNA, RNA, analogs thereof, such as LNA and PNA, and fragments thereof, possibly including non-nucleotidic or non-nucleosidic monomers, a each of which can be isolated from natural sources, recombinantly produced, or artificially synthesized. A “nucleotidic oligomer” or “oligonucleotide” as used herein refers to a polynucleotide of three or more but equal to or less than 300 nucleotides. Polynucleotides can typically be provided in single-stranded form or double-stranded form (herein also duplex form, or duplex).

A “single-stranded polynucleotide” refers to an individual string of monomers linked together through an alternating sugar phosphate backbone. In particular, the sugar of one nucleotide is bond to the phosphate of the next adjacent nucleotide by a phosphodiester bond. Depending on the sequence of the nucleotides, a single-stranded polynucleotide can have various secondary structures, such as the stem-loop or hairpin structure, through intramolecular self-base-paring. A hairpin loop or stem loop structure occurs when two regions of the same strand, usually complementary in nucleotide sequence when read in opposite directions, base-pairs to form a double helix that ends in an unpaired loop. The resulting lollipop-shaped structure is a key building block of many RNA secondary structures as will be understood by a skilled person. The term “small hairpin RNA” or “short hairpin RNA” or “shRNA” as used herein indicate a sequence of RNA that makes a tight hairpin turn and can be used to silence gene expression via RNAi.

A single strand polynucleotide has a 5′ end and a 3′ end The terms “5′ end” and “3′ end” of a single stranded polynucleotide indicate the terminal residues of the single strand polynucleotide and are distinguished based on the nature of the free group on each extremity. The 5′-end of a single strand polynucleotide designates the terminal residue of the single strand polynucleotide that has the fifth carbon in the sugar-ring of the deoxyribose or ribose at its terminus (5′ terminus). The 3′-end of a single strand polynucleotide designates the residue terminating at the hydroxyl group of the third carbon in the sugar-ring of the nucleotide or nucleoside at its terminus (3′ terminus). The 5′ end and 3′ end terminus in various cases can be modified chemically or biologically e.g. by the addition of functional groups or other compounds as will be understood by the skilled person.

A “double-stranded polynucleotide” or “duplex polynucleotide” refers to two single-stranded polynucleotides bound to each other through complementarily binding. The duplex typically has a helical structure, such as a double-stranded DNA (dsDNA) molecule or a double stranded RNA, which is maintained largely by non-covalent bonding of base pairs between the strands and by base stacking interactions. The term “5′-3′ terminal base pair” with reference to a duplex polynucleotide refers to the base pair positioned at an end of the duplex polynucleotide that is formed by the ′5 end of one single strand of the two single strand forming the duplex polynucleotide base-paired with the 3′ end of the single strand forming the duplex polynucleotide complementary to the one single strand. Accordingly a duplex polynucleotide formed by a first single strand complementarily bound to a second single strand, has two opposite ends: a first end of the duplex polynucleotide having a “5′-3′ terminal base pair” formed by the 5′ end of the first single strand and the 3′ end of the second single strand, and a second end of the duplex polynucleotide opposite to the first formed by the 5′ end of the first single strand and the 3′ end of the second single strand.

The term “complementary” as used herein indicates a property of single stranded polynucleotides in which the sequence of the constituent monomers on one strand chemically matches the sequence on another other strand to form a double stranded polynucleotide. Chemical matching indicates that the base pairs between the monomers of the single strand can be non-covalently connected via two or three hydrogen bonds with corresponding monomers in the another strand. In particular, in this application, when two polynucleotide strands, sequences or segments are noted to be complementary, this indicates that they have a sufficient number of complementary bases to form a thermodynamically stable double-stranded duplex. Double stranded of complementary single stranded polynucleotides include dsDNA, dsRNA, DNA:RNA duplexes as well as intramolecular base paring duplexes formed by complementary sequences of a single polynucleotide strand (e.g., hairpin loop).

The terms “complementary bind”, “base pair”, and “complementary base pair” as used herein with respect to nucleic acids indicates the two nucleotides on opposite polynucleotide strands or sequences that are connected via hydrogen bonds. For example, in the canonical Watson-Crick DNA base pairing, adenine (A) forms a base pair with thymine (T) and guanine (G) forms a base pair with cytosine (C). In RNA base paring, adenine (A) forms a base pair with uracil (U) and guanine (G) forms a base pair with cytosine (C). Accordingly, the term “base pairing” as used herein indicates formation of hydrogen bonds between base pairs on opposite complementary polynucleotide strands or sequences following the Watson-Crick base pairing rule as will be applied by a skilled person to provide duplex polynucleotides. Accordingly, when two polynucleotide strands, sequences or segments are noted to be binding to each other through complementarily binding or complementarily bind to each other, this indicate that a sufficient number of bases pairs forms between the two strands, sequences or segments to form a thermodynamically stable double-stranded duplex, although the duplex can contain mismatches, bulges and/or wobble base pairs as will be understood by a skilled person.

The term “thermodynamic stability” as used herein indicates a lowest energy state of a chemical system. Thermodynamic stability can be used in connection with description of two chemical entities (e.g., two molecules or portions thereof) to compare the relative energies of the chemical entities. For example, when a chemical entity is a polynucleotide, thermodynamic stability can be used in absolute terms to indicate a conformation that is at a lowest energy state, or in relative terms to describe conformations of the polynucleotide or portions thereof to identify the prevailing conformation as a result of the prevailing conformation being in a lower energy state. Thermodynamic stability can be detected using methods and techniques identifiable by a skilled person. For example, for polynucleotides thermodynamic stability can be determined based on measurement of melting temperature Tₘ, among other methods, wherein a higher Tₘ can be associated with a more thermodynamically stable chemical entity as will be understood by a skilled person. Contributors to thermodynamic stability can comprise, chemical compositions, base compositions, neighboring chemical compositions, and geometry of the chemical entity.

Nucleic acid sequences can be designed to ensure complementary and specific binding of nucleic acids to form thermodynamically stable duplexes

The wording “specific” “specifically” or “specificity” as used herein with reference to the binding of a first molecule to second molecule refers to the recognition, contact and formation of a stable complex between the first molecule and the second molecule, together with substantially less to no recognition, contact and formation of a stable complex between each of the first molecule and the second molecule with other molecules that may be present. Exemplary specific bindings are antibody-antigen interaction, cellular receptor-ligand interactions, polynucleotide hybridization, enzyme substrate interactions and additional interactions identifiable by a skilled person.

Accordingly, the wording “specific binding” in nucleic acids refers to the precise molecular recognition between two sequences that form a thermodynamically stable interactions through complementary base pairing while avoiding unwanted cross-interactions with other sequences, as will be understood by a skilled person

Various tools are available that can be used to design sequences configured to form stable complexes through specific and complementary binding of nucleic acid Exemplary software for designing stable nucleic acid duplexes, comprise NUPACK (nupack.org) which includes tools for structure prediction and stability analysis, OligoAnalyzer (idtdna.com/calc/analyzer) which provides essential features for calculating thermodynamic parameters and predicting secondary structures; DNAforge (dnaforge.org) which enables automated sequence design with molecular dynamics simulation capabilities. PFRED, available through GitHub, which offers open-source solutions for oligonucleotide design with emphasis on stability criteria, and additional tools able to perform molecular weight calculations, melting temperature predictions, secondary structure evaluation, hybridization prediction, and cross-reactivity analysis required to manufacture stable, long-lasting complexes, identifiable by a skilled person.

In embodiments of the disclosure single stranded and/or double stranded nucleic acids can form nucleic acid condensates through intermolecular interactions among different nucleic acid molecules, which under proper thermodynamic conditions herein indicated as condensing thermodynamic condition initiate formation of nucleic acid condensate via liquid-liquid phase separation as will be understood by a skilled person.

Thermodynamic conditions represent the physical and chemical parameters that govern molecular interactions within an environment through thermodynamic variables. Molecular interactions in condensate monomeric structure of the disclosure comprise nucleic acid structural transitions, ligand-target binding events, and condensate formation.

The thermodynamic parameters defining the thermodynamic conditions are: temperature, salt concentration (monovalent and divalent ions), pH of the solution, Molecular concentrations (nucleic acids, ligands, targets), Macromolecular crowding agents and Phase-separating components as will be understood by a skilled person.

The thermodynamic variables affected by the thermodynamic parameters and determining stability and specificity of molecular interactions are Gibbs free energy (ΔG°), Enthalpy (ΔH°), Entropy (ΔS°), Binding/dissociation constants (KD), Surface tension, Critical concentrations for phase separation

The relationship between the thermodynamic variables follows the fundamental equation: ΔG°=ΔH°−TΔS°ΔG°=ΔH°−TΔS° as will be understood by a skilled person This framework describes how the system moves toward equilibrium, with all molecular processes driven by the minimization of free energy under the given conditions, including the formation of distinct phases and condensates, as will be understood by a skilled person

Under thermodynamic conditions in the sense of the disclosure thermodynamic parameters affect and control the thermodynamic variables, which in turn govern the molecular interactions. In particular, molecular interactions follows a clear sequence where thermodynamic conditions, defined by physical and chemical parameters such as temperature, salt concentration, pH, molecular concentrations, crowding agents, and phase-separating components, act as primary controlling factors. These parameters set up thermodynamic conditions which directly influence the thermodynamic variables (Gibbs free energy, enthalpy, and entropy) of the system. The resulting thermodynamic variables, in turn, determine the features of molecular interactions, such as melting temperature (Tm) and overall free energy change (ΔG), which ultimately govern whether specific interactions will occur spontaneously and how stable they will be.

Accordingly, thermodynamic conditions herein describe govern the formation, stability, and dissociation of nucleic acid structures, including their ability to form condensates through liquid-liquid phase separation. These conditions determine whether nucleic acid strands will form stable duplexes, remain single-stranded, participate in more complex structural arrangements, or undergo condensation into higher-order assemblies.

As consequence in embodiments herein described by modifying the thermodynamic parameter and corresponding conditions as skilled person can predictably affect the thermodynamic variables and, consequently, control the likelihood and stability of intramolecular and intermolecular interaction interactions, whether they involve nucleic acid structural changes, ligand-target binding, or condensate formation,

In embodiments of the disclosure, intramolecular interactions and intermolecular interactions among different nucleic acids occur as a result of intermolecular forces (MFs) which are attractive or repulsive forces mediating interactions between neighboring molecules without causing significant restructuring of their electronic structure. The IMF are electrostatic in nature and operate between molecules while maintaining individual molecular identity. Exemplary IMFs comprise Permanent dipole-dipole forces, Hydrogen bonds Van der Waals forces (including Keesom, Debye, and London dispersion forces) Ion-dipole forces and π-interactions (Cation-π, π-π bonding) and additional IMFs identifiable by a skilled person

Accordingly intermolecular interactions among nucleic acids which can initiate formation of nucleic condensate comprise weak, multivalent interactions that create flexible and dynamic assemblies. such as cation-anion interactions, dipole-dipole interactions, cation-π interactions, π-π interactions, hydrophobic interactions and additional interactions identifiable by a skilled person. [6]

The weak, multivalent interaction among nucleic acids create a dynamic network where: components can freely exchange between condensed and dilute phases, the assembly maintains flexibility through transient bonds and the interactions follow a “stickers-and-spacers” model, where specific binding regions (stickers) are connected by flexible segments (spacers).

The resulting condensate forms when these collective interactions reduce the solubility of the molecules and drive the local concentration above the saturation threshold, which creates a dense phase enriched in nucleic acid herein identified as “nucleic acid condensate” surrounded by a dilute phase.

In particular, the wording, “nucleic acid condensate” in the sense of the disclosure indicates dense membraneless compartments formed by nucleic acids molecules forming intermolecular interaction when the nucleic molecules collapse from an extended state into compact, orderly particles through phase separation processes [6]

Accordingly nucleic acid condensates in the sense of the disclosure indicate a compartment structure formed by nucleic acids which lack a membrane boundary but maintain a distinct inside-outside environment through surface tension. Nucleic acid condensate can exhibit various shapes and can form complex multiphase or multilayer architectures concentrating specific nucleic acids while excluding others will be understood by a skilled person. [7]

Nucleic acid condensates in the sense of the disclosure are in liquid phased and do not comprise (1) disordered solid precipitates, typical of school demonstrations or laboratory purifications using alcohol; these exhibit no clear interface and targets could not be easily released from the precipitates (2) rigid gel-like condensates that are not subject to layering via thermal annealing or centrifugation. [8]

In embodiments herein described at least some of the nucleic acid molecules forming the nucleic acid condensate (herein indicated as “nucleic acid condensate monomers”) are further configured to form sub-compartments within the nucleic acid condensate.

In those nucleic acid condensate monomers, duplex nucleic acid arm segments presenting interaction domains or segments, forms scaffold moieties which following condensation of the nucleic acid triggered by intermolecular interactions through the interaction domain, provide a structural framework within the condensate comprising sub-compartments within the nucleic acid condensate structure.

The structure of these duplexes segment and interaction domains can be defined by the respective melting temperature Tm and ΔG at the condensing thermodynamic conditions.

The wording “melting temperature (Tm) as used herein with reference to duplex nucleic acids indicates the temperature at which 50% of the double-stranded DNA molecules have dissociated into single strands, while the other 50% remains in the double-helix form at set conditions.

The wording “melting temperature” (Tm) as used herein with reference to single-stranded nucleic acids, refers to the structural transition temperature at which 50% of the intramolecular secondary structures, such as hairpins, stem-loops, or pseudoknots, are denatured at set conditions. These secondary structures form through self-complementary base pairing within the same molecule, and their stability depends on factors like sequence composition, salt concentration, and the length of complementary regions.

Key features affecting the melting temperature of a nucleic acid comprise GC content (higher GC content leads to higher melting temperatures due to the stronger bonding between G and C bases). DNA Length (Longer nucleic acid molecules require more energy to dissociate and therefore have higher melting temperatures than shorter sequences) ionic strength of the nucleic acid solution (higher ionic strength results in higher melting temperatures)

The melting temperature (Tm) of nucleic acids at set condition can be measured through UV spectrophotometry: which measures absorbance changes at 260 nm as a function of temperature and detects transition of nucleic acid from single stranded form to double stranded form and vice versa, as the single-stranded nucleic acids have higher absorbance than double-stranded forms

Accordingly, using a UV-Vis spectrophotometer, the temperature can be gradually increased while monitoring absorbance changes to determine the point where 50% of the molecules are denatured to determine the Tm of a duplex nucleic acid For single-stranded nucleic acids, UV spectrophotometry at 260 nm can detect the unstacking of bases that occurs during the structural transition to reveal the temperature at which 50% of the intramolecular secondary structures have denatured.

An additional method to measure Tm of a nucleic acid is provided by the High resolution melting (HRM) techniques which offers high sensitivity and can be used for measuring Tm of various nucleic acid structures including duplexes, triplexes, and hairpins. HRM provides comparable results to UV melting methods when the Tm is above 40° C. [9]. Additional methods are identifiable by a skilled person and include theoretical calculations which can calculate Tm with a margin of error of ±2-3 C with respect to a corresponding Tm measured as will also be understood by a skilled person

The term ΔG as used herein indicates the thermodynamic variable Gibbs free energy change (ΔG) which represents the maximum amount of non-volume expansion work that can be extracted from a closed system at constant temperature and pressure. It is mathematically defined by the equation ΔG=ΔH−TΔS, where ΔH is the change in enthalpy, T is the temperature in Kelvin, and ΔS is the change in entropy

ΔG can be determined through measurements of other thermodynamic variables. The most common approaches involve either calorimetric measurements to determine ΔH and ΔS, or equilibrium measurements to calculate ΔG through the relationship ΔG=−RTInK, where R is the gas constant, Tis temperature, and K is the equilibrium constant. For systems at constant temperature, ΔG can also be calculated by measuring volume changes as pressure varies, using the integral relationship ΔG=∫VdP

ΔG is particularly useful in predicting the direction of chemical reactions and physical processes under set thermodynamic conditions. A negative ΔG indicates that a process is thermodynamically favorable and will occur spontaneously, while a positive ΔG indicates that the process is unfavorable and requires energy input, as will be understood by a skilled person

In nucleic acid condensate monomers herein described forming condensate in each duplex arm segment each strand complementarily binds to the first arm domain of a second different strand of the nucleic acid condensate monomer at the condensing thermodynamic conditions with a ΔGₛ, to form the duplex arms segments of the functionalized nucleic acid nanostructure. Each duplex arm segments has a melting temperature Tmₛ as will be understood by a skilled person

In nucleic acid condensate monomers herein described forming condensate with sub-compartments, each interaction domains presented on a duplex arm segment independently has a melting temperature Tmᵢ and is configured to form at the condensing thermodynamic conditions intermolecular interactions with an interaction domain of a different nucleic acid nanostructure. having a ΔGᵢ,

in the nucleic acid condensate monomer of the present disclosure which forms sub-compartments within the condensate, at the condensing thermodynamic conditions Tmₛ, is at least 10° C. higher than Tmᵢ, and ΔGₛ is at least twice ΔGᵢ. In monomers with this configuration the arm duplexes will provide a scaffold forming a structural framework of the condensate comprising compartments held together by the MF resulting from the interaction of the interaction domains among each other as will be understood by a skilled person

In particular in embodiments herein described, under condensing thermodynamic conditions, intermolecular interactions among nucleic acid condensate monomers of the present disclosure, results in sharp phase transition and in a thermodynamic process involving nucleation, growth, and coarsening when the nucleic acid condensate monomer concentration exceeds the saturation threshold forming a condensate comprising sub-compartments as will be understood by a skilled person

In embodiments herein described nucleic acid condensate monomers can be engineered and configured to comprise duplexes arm segments providing scaffold moieties for the condensate and interaction domains or segments configured to form condensate with sub-compartments.

The term “domain” in the sense of the present disclosure indicates a part of a given nucleic acid having a structure specifically associated with a function and that exist independently of the rest of the polynucleotide. The structure/function association in a domain is typically conserved during the chemical and/or biological reaction associated with the nucleic acid.

The term “segment” as used herein indicates a portion of a construct having chemical and/or biological properties that are functional to the chemical and/or biological properties of the entire construct as a whole. The term “segment” as used herein in connection with a nucleic acid condensate monomer, thus indicates a portion of a nucleic acid condensate monomer having chemical and/or biological properties that are functional to changes in conformation of the nucleic acid condensate monomer construct, and/or to a related ability to perform the enzyme assisted release herein described.

Nucleic acid condensation monomers herein described can comprise n nucleic acid strands with n being an integer selected from 1 to 12, each nucleic acid strands comprising at least two arm domains ranging from 5 to 75 nucleotides and attached to one another optionally through a flexible linker domain ranging from 1 to 2 nucleotides in length.

In the nucleic acid condensate monomer at least one nucleic acid strand of the n nucleic acid strand further comprises an interaction domain ranging from 1 to 20 nucleotides in length attached to a terminus of an arm domain.

A “terminus” or “end” in nucleic acids refers to either end of a molecule, with each terminus comprising a single nucleotide. In nucleic acid herein described the 5′ terminus is named for the carbon atom at position 5 of the terminal nucleotide's sugar ring, while the 3′ terminus is named for the carbon atom at position 3 of the terminal sugar ring. Accordingly, in single-stranded nucleic acids, there is one 5′ end presenting a phosphate group and one 3′ end presenting a hydroxyl group. In double stranded nucleic acid, n double-stranded nucleic acids, there are four termini: two 5′ ends and two 3′ ends, one of each on both strands as will be understood by a skilled person.

Exemplary interaction domains comprise, single stranded ‘sticky’ ends independently ranging from 1 to 15 nucleotides in length presented on the 5′end or 3′end of an arm domain,

A further exemplary interaction domain is a single strand nucleic acid ranging from 4 to 40 nucleotides in length having a 5′ attached to the 3′ end of a first arm domain and the 3′ end attached to the 5′ end of a second arm domain to form a loop of a resulting a stem loop structure. In an interacting domain of the disclosure forming part of a nucleic acid loop up to 40 nucleotides in total length, 4-15 nucleotide forming interacting domain presented for interaction, and 0-25 nucleotide are flexible linkers as will be understood by a skilled person.

Another exemplary interaction domain is a double stranded strand nucleic acid formed by a single pair of nucleotides attached to the 3′ end of a first arm domain and 5′ end of a second arm domain as will be understood by a skilled person

In the functionalized nucleic acid condensate monomer, each of the arm domains, each of the interaction domain, and optional linker domain has sequences orthogonal to other sequences of the functionalized nucleic acid monomer and each arm domain complementarily binds to another arm domain of a same or different strand at the condensing thermodynamic conditions with a ΔGₛ to form a duplex arm segments ranging from 5 to 75 nucleotides in length and having a melting temperature Tmₛ

In the nucleic acid condensate monomer the n nucleic acids comprise at least six arm domains in a same or different strands which complementarily bind to one another at the condensing thermodynamic conditions form at least three duplex arm segments each configured to form a scaffold for the assembly of the condensate,

In the nucleic acid condensate monomer, each duplex arm segment presents at a terminus an interaction domain having a melting temperature Tmᵢ, In the functionalized nucleic acid condensate monomer, each interaction domain is configured to form intermolecular interactions with at least one interaction domain of a different nucleic acid monomer, the intermolecular interactions having a ΔGᵢ and configured to initiate under the condensing thermodynamic conditions the liquid-liquid phase separation of the functionalized nucleic acid condensate

In the nucleic acid condensate monomer at the condensing thermodynamic conditions Tmₛ, is at least 10° C. higher than Tmᵢ, and ΔGₛ is at least twice ΔGᵢ to trigger formation of a condensate with sub-compartment at the condensing thermodynamic conditions.

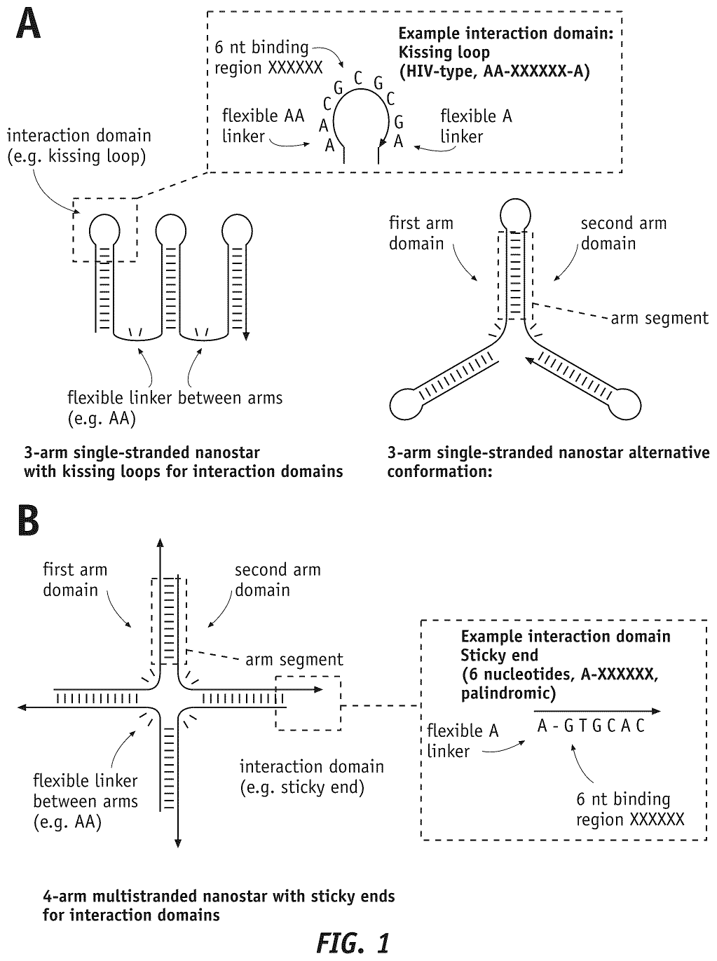

A schematic illustration of exemplary configurations of the arm domain, interaction domain and optional linker domain are shown in FIGS. 1A and 1B.

In particular in FIG. 1A, a single stranded nucleic acid condensate monomer is described which comprises three duplex arm segments attached to one another by 2 nt linker domains formed by unpaired sequences. In the schematic illustration of FIG. 1A each duplex arm segment is formed by secondary structures formed by six arm domains within the single nucleic acid which complementary bind to form the respective three duplex arm segments of the nucleic acid condensate monomers.

FIG. 1A shows a single-stranded 3-arm DNA nanostar condensate monomer. The monomer is termed “single-stranded” because it comprises a single strand. It can be comprised of DNA or RNA. When comprised of RNA the single strand can be made by RNA transcription from a DNA template. At left is a linear open representation, at right is a more closed, star-like representation of an alternative conformation that the monomer can assume. While the monomer is called single stranded because of its composition, it nevertheless has secondary structure which comprise double-stranded segments. The double-stranded arm segments provide a structural scaffold for the interaction domains, of which there must be at least three. The arm domains are shown as 12 nucleotides long, but they can range from 5 to 75 nucleotides. A flexible linker is incorporated between arm domains (here two unpaired nucleotides, ‘AA’). A small gray dashed box shows one of the three interaction domains. The large gray dashed box shows an example interaction domain, in this case an HIV-type 9 nucleotide kissing loop that comprises two flexible linkers and the binding region arranged 5′ AA-XXXXXX-A 3′, which would be typically used on an RNA monomer. When only one condensate monomer is used to build a condensate layer, XXXXXX is a self-complementary palindrome. However when two condensate monomers are used to build a condensate, then a first XXXXX on the first condensate is complementary to a second XXXXXX on the second condensate.

In the schematic illustration of FIG. 1A, each of the three duplex arm segments presents an interaction domain, and in particular, the duplex arm segments present a nucleic acid loop interaction domain.

A multistranded nucleic acid condensate monomer is described in FIG. 1B, where four nucleic acid strands are complementarily bound to form a nucleic acid nanostar structure. In the schematic illustration of FIG. 1B, each nucleic acid strands comprise a first arm domain and a second arm domain attached to one another by a flexible linker domain formed by 2 unpaired nucleotide sequences and presenting an interaction domain at a terminus.

In particular, the schematic illustration of FIG. 1B, shows an exemplification of the configuration of each strand of the nucleic acid nanostar structure in which the 5′end of the second arm domain is covalently attached to the 3′ end of first arm domain, optionally via the linker domain. In each strand, the 5′ end of one of the interaction domain is covalently attached to the 3′ end of the second arm domain, or in the alternative the 3′ end of the interaction domain is covalently attached to the 5′ of the first arm domain as will be understood by a skilled person upon reading of the disclosure.

FIG. 1B shows a multistranded 4-arm DNA nanostar condensate monomer. The monomer is termed “multistranded” because it comprises more than one strand. The arm domains are shown as 12 nucleotides long, but it can range from 5 to 75 nucleotides. The double-stranded arm segments provide a structural scaffold for the interaction domains, of which there must be at least three. Every strand has a first arm domain and a second arm domain. The first arm domain of one strand binds and forms a duplex with the corresponding second arm domain of a neighboring strand. A flexible linker is incorporated between arm domains (here two unpaired nucleotides, ‘AA’). A small gray dashed box shows one of the four interaction domains. The large gray dashed box shows an example interaction domain, in this case a sticky end that comprises one flexible linker and the binding region arranged 5′ A-XXXXXX-3′, which would be typically used on an DNA monomer. When only one condensate monomer is used to build a condensate layer, XXXXXX is a self-complementary palindrome. However when two condensate monomers are used to build a condensate, then a first XXXXX on the first condensate monomer is complementary to a second and different XXXXXX on the second condensate monomer.

The term “attach” “bind” or “attached” “bound” as used herein, refers to connecting or uniting by a bond, link, force, or tie in order to keep two or more components together, which encompasses either direct or indirect attachment. For example, “direct attachment” refers to a first molecule directly bound to a second molecule or material, while “indirect attachment” in refers to one or more intermediate molecules being disposed between the first molecule and the second molecule or material.

The term “covalent attachment” “covalent binding” or “covalently linked” as used herein indicates connection between two segments through formation of a chemical bonding that is characterized by sharing of pairs of electrons between atoms, known as the covalent bond. Examples of covalent binding can include, covalent bonds formed between any two of the following: RNA or portions RNA, DNA or portions of DNA, any nucleotide or derivative thereof, and/or enzyme.

The schematic illustration of FIG. 1B also shows that each of at least four nucleic acid strand of the functionalized nucleic acid nanostar structure exemplified in in FIG. 1B, the 5′ end of one of the first arm domain or the second domain of the strand, the 3′ end of the interaction domain.

In the nucleic acid nanostructure schematically illustrated in FIG. 1B, the interaction among domains of a condensate nanostructure is further exemplified. In particular in schematic illustration of FIG. 1B it is shown that the first arm domain of each strand complementarily binds the second arm domain of a different strand of the monomer to form four duplex arm segments each presenting an interaction domain formed by sticky ends. In the schematic illustration of FIG. 1B.

The configuration exemplified in FIG. 1B is representative of additional nucleic acid nanostar structures of the disclosure which can be formed by 3 to 12 strands, each strand comprising a first arm domain, a second arm domain, a linker domain and one of an interaction domain and a targeting domain, each domain having a 5′end and a 3′ end.

In a nucleic acid nanostar structure of the disclosure, the first and second arm domains of each strand have the function of specifically and complementarily binding the second and first arm domain of another strand of the same monomer, respectively, thus forming a duplex arm segment of the nucleic acid condensate monomer. The first arm domain of a strand of the nanostructure also has the function of presenting an interaction domain for binding with interactions domain of other nucleic acid condensate as it will be understood by a skilled person.

In a nucleic acid condensate monomers schematically exemplified in FIG. 1A and FIG. 1B, optional linker domains in each strand are interposed and connects the first arm domain and the second arm domain of that strand but can be absent in the corresponding structures as will be understood by a skilled person.

In a nucleic acid condensate monomers schematically exemplified in FIG. 1A and FIG. 1B a linker segment formed by three or more linker domains indicates the central branching point where multiple duplex nucleic acid arms intersect. This linker segment serves as the core architectural element that determines the overall geometry and structural properties of the condensate monomer in particular when the condensate monomer is formed by nanostar. In embodiments, where the nucleic acid condensate monomer comprises a nanostar, the linker segment conformation is defined by specific angles between the arms, with values ranging between 0 and 180 degrees.

In a typical four-arm nanostar exemplified by the schematic illustration of FIG. 1B, the linker segments connect multiple double-helical arms that extend outward from this central point, each typically ending in binding domains such as sticky ends or kissing loops The linker domain's flexibility plays a crucial role in the nanostar's overall stability and function as will be understood by a skilled person. While single branched linkers tend to be relatively flexible, their rigidity can be enhanced through various design strategies as will be understood by a skilled person.

In a nucleic acid condensate monomers schematically exemplified in FIGS. 1A to 1B the interaction domain of each strand is orthogonal to other sequences of the functionalized nucleic acid condensate monomer and is configured to form intermolecular interactions with an interaction domain of a different nucleic acid nanostructure. under condensing thermodynamic conditions to initiate a liquid-liquid phase separation of the nucleic acid nanostructure in a functionalized nucleic acid condensate having a distinct density

In a nucleic acid condensate monomers schematically exemplified in FIGS. 1A to 1B arm domains, duplex arm segments and interaction domains/segments and linker domains have a same length A skilled person will understand however that the length of the different domain and segments can be different as long as the Tmₛ, of each arm duplex segment is at least 10° C. higher than the Tmᵢ of each interaction domain, and the ΔGₛ of the complementary binding of the strand in each arm duplex segment is at least twice of the ΔG; of the interactions between interactions domain of different monomers.

In any one of the nucleic acid condensate monomers schematically exemplified in FIGS. 1A to 1B the interaction domains and the optional linker domain have a sequence orthogonal to other sequences of the n strands of the nucleic acid monomers,

The term “orthogonal” and “orthogonality” as used herein with respect to nucleic acid refer to the property of nucleic sequences that allows them to interact exclusively with their intended complementary partners while avoiding cross-hybridization with other sequences present in the same environment. This characteristic is used in sequence design, where nucleic acid strands maintain specificity in binding interactions and avoid unwanted base-pairing with non-target sequences, and encompass not only base-pairing but also broader structural and thermodynamic properties of nucleic acid molecules. Orthogonal nucleic acids are configured so that each nucleic acid component operates independently while maintaining its intended structure and function. An example orthogonal nucleic acid design is provided DNA origami, where hundreds of unique “staple” strands specifically bind to their intended targets on a scaffold strand without cross-hybridizing as will be understood by a skilled person.

In any one of the nucleic acid condensate monomers schematically exemplified in FIG. 1A and FIG. 1B, the configuration of the domains of each strand is directed to and drives formation of the condensate monomer through complementary binding of related sequence In particular, the duplex arm segments of each condensate monomer form an n number of arms of the nucleic acid nanostructure of each condensate monomer defining the valency of the condensate monomer.

The term “valency” as used herein with respect to nucleic acid condensates represents the average number of binding partners or interaction sites that a molecule can simultaneously engage with. Valency is a result effective variable in determining both the formation and architecture of condensed phases as well as the number of binding partners. The number of binding partners serves as an efficient control mechanism for condensate formation, where reducing monomer valency can effectively regulate assembly and disassembly processes in a more accurate and effective manner than adjusting total component concentrations. Valency directly influences the internal organization of condensates, affecting their structural layering and the spatial distribution of components, ultimately determining whether a system forms single multilayered condensates or multiple non-interacting condensates with varying compositions, as will be understood by a skilled person upon reading of the present disclosure.

In a nucleic acid condensate monomer, the valency of the as well as their sequence and length are also functional to control the density of the condensate as will be understood by a skilled person upon reading of the present disclosure.

Accordingly a nucleic acid condensate monomers schematically exemplified in FIGS. 1A to 1B and in particular in a nanostar structure exemplified by FIG. 1B, the duplex arm segments of the nucleic acid nanostructure as the function to provide the valency of the condensate monomer as well as presenting the interaction domain for binding to other monomer of the condensate thus affecting the formation and architecture of condensed phases as well as the number of binding partners as will be understood by a skilled person upon reading of the disclosure.

In some embodiments the nucleic acid condensate monomers can composed of DNA, RNA, LNA, PNA, or any analog of a nucleic acid. In some embodiments nucleic acid condensate monomers are hybrids of two or more types of nucleic acid or nucleic acid analogs, for example DNA/RNA hybrids, DNA/PNA hybrids, DNA/LNA, or DNA/RNA/PNA hybrids as will be understood by a skilled person.

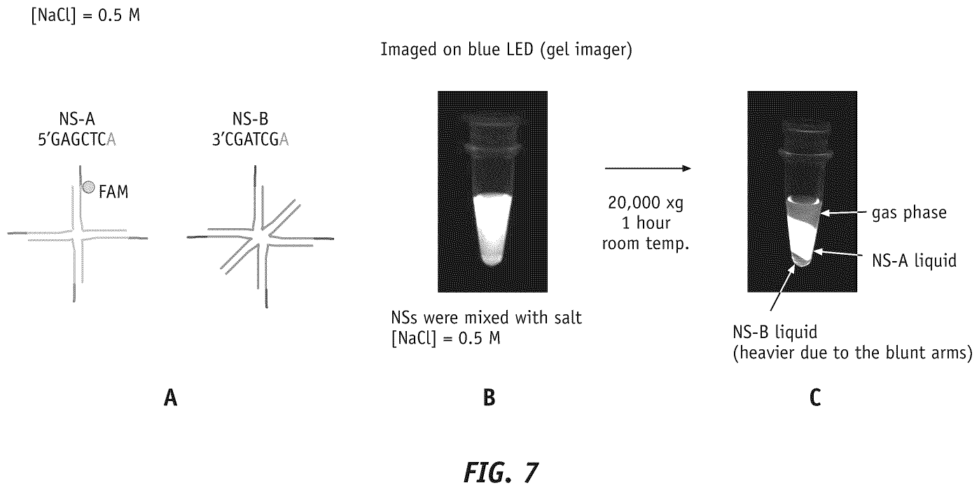

In some embodiments, the nucleic acid structure of the functionalized nucleic acid condensate monomer, each of the duplex arm segment of a nanostructure present an interaction domain for binding and forming of the condensate. In some embodiments, the nucleic acid structure of the functionalized nucleic acid condensate monomer, one or more duplex arm segments present no interaction domain (see Example 4, FIG. 7).

In embodiments, herein described nucleic acid condensate monomers comprising nanostar structure as well as single stranded or double stranded condensate monomer are engineered and synthesized so that they can condense, under condensing thermodynamic conditions, to form a liquid or gel phase that separates readily from a complex bulk mixture having a set density.

The term “density” when used herein with respect to a nucleic acid condensate, indicates a complex biophysical property that describes both the mass concentration and molecular distribution within these biomolecular assemblies. In the context of condensates, density manifests as the spatial organization of nucleic acids and associated proteins within a defined volume, typically measured through refractive index imaging techniques. This property reflects how tightly packed the molecular components are within the condensate compared to their surrounding environment. Accordingly, a skilled person will understand that the density within nucleic acid condensates is not static but rather represents a dynamic equilibrium influenced by nucleic acid-mediated interactions, multivalent cations, and the environment. This property becomes particularly evident when the nucleic acid transitions from an extended state to a condensed form, similar to the packaging observed in bacterial viruses and sperm cells. Thus the resulting density distribution reflects the balance between molecular interactions and environmental conditions that maintain the condensate's structure and function as will be understood by a skilled person.

A skilled person will understand that the density of a particular nucleic acid condensate is increases with increasing concentration of the nucleic acid material in that particular condensate. The density of a particular nucleic acid condensate increases with higher valency of the condensate monomers, the arm number, and/or the average arm number of the condensate monomer. The density of a particular nucleic acid condensate is decreases with increasing of the length of the duplex arm domain of the condensate monomers. A skilled person will also understand that the density of a particular nucleic acid condensate decreases with increasing the ΔGᵢ of the interactions among interactions domains of different monomers of the condensate. In particular increasing dGi is “weakening the strength of the binding of the interaction domains as will be understood by a skilled person.

A skilled person will understand that the density of a particular nucleic acid condensate can be related to the concentration of the nucleic acid condensate monomers in that particular condensate, using the molecular weight of the nanostar and well-known properties of DNA. Therefore a skilled person understands that measurements of trends in nanostar concentration (increasing or decreasing) as a function of a structural feature of a condensate reflect and imply a similar trend in density as a function of that same structural feature.

A skilled person will understand that if all structural features of a condensate monomer are fixed, and one is varied, a trend in density as a function of that structural feature can be measured and further used for designing condensates and ordered layers of condensates. In particular, a skilled person understands that if all the structural features of a particular nucleic acid condensate monomer are fixed, except for the valency (where valency is the number of arms having interaction domains), and the valency is increased, then the density of the corresponding condensate will increase. A skilled person understands that parallel trends hold for other structural features of condensate monomers. The density of a condensate corresponding to a particular monomer also increases with increasing arm number, or with increasing average arm number (again holding all other structural features fixed). A skilled person will understand that the density of condensate corresponding to a particular nucleic acid condensate monomer will increase as the length of the duplex arm domain of the monomer is decreased. A skilled person will also understand that the density of a condensate corresponding to a particular nucleic acid monomer will increase as the ΔGᵢ of the interactions among interactions domains of different monomers of the condensate is decreased, that is, as the interactions are strengthened.

Therefore, in nucleic acid condensates of the present disclosure the density can be increased by increasing the valency of the condensate monomers used, increasing the arm number of the condensate monomers used, increasing the average arm number of the condensate monomers used, by decreasing the length of the duplex arm segments in the monomers used to form the condensate and/or by decreasing the ΔGᵢ of the interactions among interactions domains of different monomers (strengthening the interaction between monomers) of the condensate as will be understood by a skilled person.

A skilled person will understand that in a condensate layer having a single condensate monomer or different condensate monomer having a same valence, same length of the duplex arm segments and interaction domains forming intermolecular interactions at a same ΔGᵢ The density of a nucleic acid condensate can be expressed through nucleic acid mass concentration, as will be understood by a skilled person.

Similarly in a condensate layer having a single condensate monomer or different condensate monomer having a same concentration, increase in valence, increase in arm number, increase in average arm number, decrease in the length of the duplex arm segments and/or decrease of ΔGᵢ of intermolecular interactions of interaction domains can be performed to increase density of a nucleic acid condensate as will be understood by a skilled person.

Several methods can be used to measure the density of nucleic acid condensates according to wet bench approaches,

An exemplary method to determine density through wet-bench techniques comprise by Refractive Index Imaging (RIIF) which provides a quantitative measure of mass concentration within condensates and enables three-dimensional distribution measurements of the refractive index, allowing researchers to determine total biomolecular density,

A further exemplary method is provided by Partition Ratio Analysis calculated as the ratio of concentration inside versus outside the condensate which is typically performed using fluorescence microscopy,

Another exemplary method is provided by Fluorescence Correlation Spectroscopy FCS, which offers precise measurements of local density within condensates, with a spatial resolution below 1 μm, making it particularly useful for examining density variations within sub-compartments and

An additional exemplary method is provided Permeability Assessment by analyzing the permeability of condensates to fluorescent protein probes, leveraging the higher accessibility of lower density condensates to probe molecules, and the stronger exclusion patterns to probe molecules of denser patter as will be understood by a skilled person

Several software tools are also available for designing NA nanostars for creating condensate-forming structures as will be understood by a skilled user. Software such as TIAMAT enables creating and modifying individual nucleic acid strands, specifying strand parameters, and designing both single and double-stranded regions [10]. DNAforge offers enables the design of complex wireframe nanostructures, generating full 3D nucleotide models and complete stapling arrangements [11], ox View allows visualization and analysis capable of handling structures with over a million nucleotides while enabling basic editing and simulation trajectory visualization [12] A typical design workflow involves creating an initial 3D mesh model using standard modeling software, converting it using one of these specialized tools, generating the routing model and nucleotide structure, and finally optimizing the design through visualization and simulation tools. The selection of software ultimately depends on specific design requirements, particularly regarding junction domain configuration, arm length, and overhang sequences, which are crucial parameters for condensate formation and stability

The flexible nature of the intermolecular interactions among nucleic acid condensate monomers enables a selective recruitment and partitioning of specific biomolecular target within a nucleic acid condensate as a result of the interplay of multiple molecular forces and interactions that drive both formation and selective partitioning of components through thermodynamic processes as will be understood by a skilled person In particular, when molecular encounter the condensate environment creates a distinct physicochemical space that selectively concentrates specific molecules while excluding others, driven by both entropic and enthalpic contributions. This dynamic system maintains a balance between stability and fluidity, allowing continuous exchange between the condensate and its surroundings while preserving its functional compartmentalization as will be understood by a skilled person. An exemplary illustration of the compartmentalization of molecules condensate formed by unfunctionalized nucleic acid compartments is illustrated by Example 2

In embodiments herein described the nucleic acid condensate monomer structures are engineered to capture biomolecular targets following functionalization of the monomer with ligands configured to specifically bind the biomolecular targets.

The term “target” as used herein indicates an analyte of interest. The term “analyte” refers to a substance, compound, moiety, or component whose presence or absence in a sample is to be detected. Analytes comprise biomolecules and in particular biomarkers as well as related complexes and cells.

Accordingly, the term “biomolecular target” as used herein indicates a substance, compound or component associated with a biological environment including but not limited to sugars, amino acids, peptides, proteins, oligonucleotides, polynucleotides, polypeptides, organic molecules, haptens, epitopes, biological cells, parts of biological cells, vitamins, hormones and the like. The term “biomarker” indicates a biomolecular target that is associated with a specific state of a biological environment including but not limited to a phase of cellular cycle, health and disease state. The presence, absence, reduction, upregulation of the biomarker is associated with and is indicative of a particular state. The “biological environment” refers to any biological setting, including, for example, ecosystems, orders, families, genera, species, subspecies, organisms, tissues, cells, viruses, organelles, cellular substructures, prions, and samples of biological origin.

The term “ligand” in the sense of the disclosure indicate is a moiety configured to form a complex with a biomolecular target by attaching the biomolecular target or portion thereof. This interaction relies on specific molecular forces, including ionic bonds, hydrogen bonds, and Van der Waals interactions. The strength and duration of this interaction depend on binding affinity, which is determined by both the chemical properties of the ligand-protein pair and the surrounding cellular environment.

Ligand in the sense of the disclosure comprise, proteins, peptides oligonucleotides and any further moiety which can be attached to a nucleic acid and specifically bind a target as will be understood by a skilled person

In some embodiments the ligands (herein also affinity reagents, binding partners) for the target molecules are small molecules, aptamers, antibodies, nanobodies, or DARPins. In some embodiments the ligands are aptamers composed of DNA, RNA, or other nucleic acid analogs. In some embodiments the ligands are aptamers that have been chemically modified (e.g., to have enhanced binding affinity or specificity to targets).

The term “small molecule” as used herein indicates an organic compound that is of synthetic or biological origin and that, although might include monomers and/or primary metabolites, is not a polymer. In particular, small molecules can comprise molecules that are not protein or nucleic acids, which play a biological role that is endogenous (e.g. inhibition or activation of a target) or exogenous (e.g. cell signaling), which are used as a tool in molecular biology, or which are suitable as drugs in medicine. Small molecules can also have no relationship to natural biological molecules. Typically, small molecules have a molar mass lower than 1 kg-mol-1. Exemplary small molecules include secondary metabolites (such as actinomycin-D), certain antiviral drugs (such as amantadine and rimantadine), teratogens and carcinogens (such as phorbol 12-myristate 13-acetate), natural products (such as penicillin, morphine and paclitaxel) and additional molecules identifiable by a skilled person upon reading of the present disclosure.

The term “aptamers” as used here indicates oligonucleic acid or peptide molecules that bind a specific target. In particular, nucleic acid aptamers can comprise, for example, nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues and organisms. Aptamers are useful in biotechnological and therapeutic applications as they offer molecular recognition properties that rival that of the antibodies. Peptide aptamers are peptides that are designed to specifically bind to and interfere with protein-protein interactions inside cells. In particular, peptide aptamers can be derived, for example, according to a selection strategy that is derived from the yeast two-hybrid (Y2H) system. In particular, according to this strategy, a variable peptide aptamer loop attached to a transcription factor binding domain is screened against the target protein attached to a transcription factor activating domain. In vivo binding of the peptide aptamer to its target via this selection strategy is detected as expression of a downstream yeast marker gene.

The terms “peptide” and “oligopeptide” usually indicate a polypeptide with less than 50 amino acid monomers, wherein the term “polypeptide” as used herein indicates an organic linear, circular, or branched polymer composed of two or more amino acid monomers and/or analogs thereof. The term “polypeptide” includes amino acid polymers of any length including full length proteins and peptides, as well as analogs and fragments thereof. As used herein the term “amino acid”, “amino acidic monomer”, or “amino acid residue” refers to any of the twenty naturally occurring amino acids, non-natural amino acids, and artificial amino acids and includes both D an L optical isomers. In particular, non-natural amino acids include D-stereoisomers of naturally occurring amino acids (these including useful ligand building blocks because they are not susceptible to enzymatic degradation). The term “artificial amino acids” indicate molecules that can be readily coupled together using standard amino acid coupling chemistry, but with molecular structures that do not resemble the naturally occurring amino acids. The term “amino acid analog” refers to an amino acid in which one or more individual atoms have been replaced, either with a different atom, isotope, or with a different functional group but is otherwise identical to original amino acid from which the analog is derived.

In functionalized nucleic acid monomer of the disclosure, a ligand forms part and is presented on a targeting domain of one or more nucleic acid strands forming the nucleic acid condensate monomer.

The wording “targeting domain” as used herein indicates a domain of a nucleic acid associated with the function of binding with a predetermined biomolecular target under set conditions.

In particular, attachment of a ligand to a targeting domain of a functionalized nucleic acid condensate monomer is performed so that the ligand can be presented for specific binding to a biomolecular target, and the ligand and/or the biomolecular target can then be released from the functionalized nucleic acid condensate monomer in a controlled manner.

The term “present” as used herein with reference to a compound or functional group indicates attachment performed to maintain the chemical reactivity of a compound or moiety as attached. Accordingly, a ligand presented on residue, a segment, or a molecule is able to perform under the appropriate conditions the one or more chemical reactions that chemically characterize the ligand, will be understood by a skilled person. Ligands in the sense of the disclosure are presented on a targeting domain of the functionalized nucleic acid condensate monomer.

The term “release” as used herein in connection with release of a biomolecular target from a corresponding ligand of a functionalized nucleic acid monomer condensate indicates the process where the bound target dissociates or separates from the ligand or the targeting domain after recognition or cleavage events. En exemplar exemplary release process comprise recognition and complementary binding to a guide strand of a of a targeting domain or nucleic acid ligand presented on the targeting domain performed by release polynucleotide. A further exemplary release process comprise the specific cleavage of a bond between ligand and biomolecular target or between the ligand and targeting domain resulting in an enzyme assisted release of the biomolecular target. A further exemplary release process comprises increasing the salt concentration in the condensate to at concentration at which the ligand-target concentration is significantly weakened. This exemplary release process can be performed on a single nucleic acid condensate separation system, or on single condensate that has been mechanically removed from a layered condensate having more than one condensate.

Accordingly, in embodiments of functionalized nucleic acid monomers herein described attachment of a ligand to a targeting domain of the functionalized nucleic acid monomer can be performed by direct or indirect covalent attachment of the ligand.

In functionalized nucleic acid condensate monomers of the present disclosure, each nucleic acid condensate monomer has a melting temperature Tmₘ in an aqueous solution. In some embodiments the aqueous solution comprises one or more salts at a concentration of 50 mM to 1M and at least one duplex arm segment of at least one nucleic acid condensate monomer of the set, comprises a targeting domain attached to and presenting a ligand configured to specifically bind the biomolecular target in the aqueous solution, at a ligand-target binding temperature Tb which is least 10° C. lower than Tmₘ

A skilled person will understand that Tb thus defines an operating temperature, selected based on both the working conditions and the intended outcome for the target captured within the condensate. A skilled person will understand that Tb not only affects the condensate behavior but also determines whether the captured target maintains its functional properties and original conformational state. The temperature selection can be performed considering condensate formation conditions, ligand-target binding conditions and desired characteristics of the retrieved target material. A skilled person will also understand that Tb is not equal to and not defined as the Tm of ligand-target binding.

Accordingly given a biomolecular target and a selected ligand a skilled person will be able to identify sequences and configuration of the monomer which can provide a Tmₘ least 10° C. higher of ligand-target binding temperature Tb of the selected target-ligand pair.

In some embodiments Tb can at least 4° C. and Tm at least 14° C., and in particular Tb ranges from 4° C. to 52° and Tm ranges from 14° C. to 65° C.

In some embodiments the aqueous solution comprises one or more monovalent salts such as sodium or potassium at a concentration of 50 to 500 mM, from 50 to 200 or from 50 to 100 mM and in some embodiments can comprise at least one Mg salt.

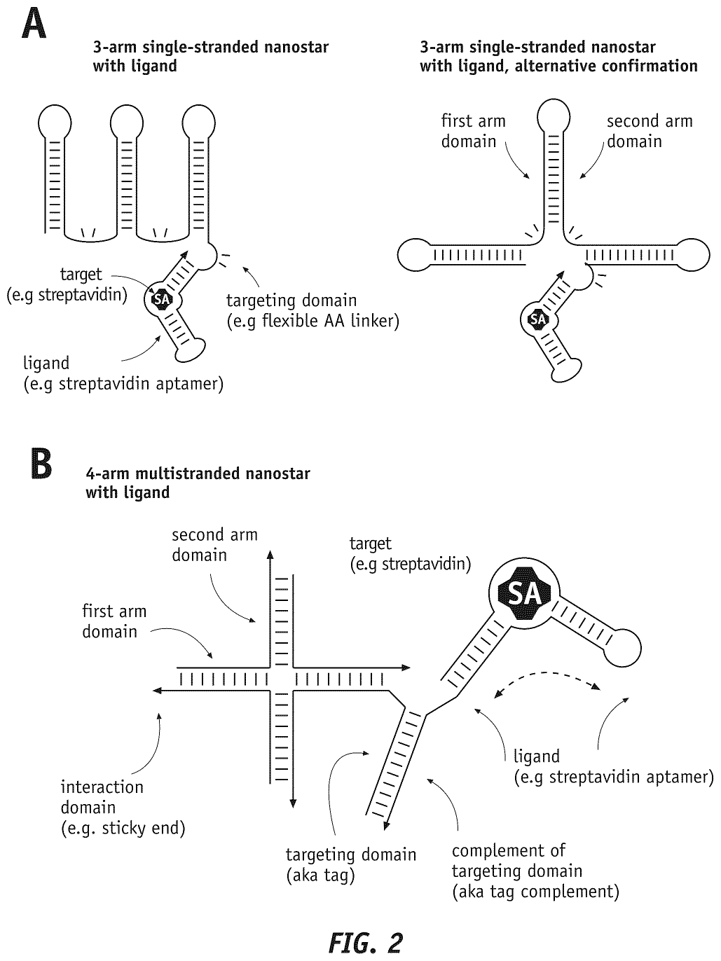

Reference is made to the schematic illustrations of FIGS. 2A to 2B which show nucleotide condensate monomer structures corresponding to the exemplary structurers schematically illustrated in FIGS. 1A to 1B and functionalized with an exemplary nucleic acid aptamer ligand binding an exemplary target biomolecule, (see Example 6)

In particular, the schematic illustration of FIGS. 2A to 2B shows respectively the single stranded condensate monomer of FIG. 1A, and the multistranded nanostar structure condensate monomer of FIG. 1B, each modified to include a targeting domain covalently attached at the 5′end of a strand of a duplex arm segment further presenting at the 3′ end of the complementary strand at stick end interaction domain.

FIG. 2A shows a single-stranded 3-arm DNA nanostar monomer in which the 3′ end of the strand has been extended with a targeting domain, which here is a flexible two nucleotide linker. Attached to the 3′ end of the targeting domain is a ligand, which here as an example, is a streptavidin aptamer.

FIG. 2B shows a multistranded 4-arm DNA nanostar monomer in which the 5′ end of one of the strands has been extended with a targeting domain. The targeting domain is shown as having 11 nucleotides, but it can range up to 50 nucleotides in length. The ligand (here a streptavidin aptamer) carries a domain that is complementary to the targeting domain, so that the targeting domain and the complement of the targeting domain form a duplex, and the ligand is attached to the condensate monomer.

Different configurations of the targeting domain are possible as will be understood by a skilled persons upon reading of the present disclosure. In particular a targeting domain can be attached to or form part of a region of an arm domain located at a distance equal to or higher than 10 nucleotides from a terminus of the arm domain which attaches another arm domain, optionally through a flexible linker.

In those embodiments, nucleic acid strand can be modified for the inclusion of a targeting domain comprising a suitable ligand, with the targeting domain separated from an adjacent arm domain or linker domain if present by at least 10 nucleotides minimum number of base pairs that serve as a spacer. For example, the targeting domain may be terminal and appended upstream of the 3′ or 5′ end of the interaction domain of a nucleic acid strand. Alternatively, the targeting domain can be placed in the middle of a nucleic acid strand, separated from adjacent arm domain or linker domain if present by at least a minimum 10 nucleotides that serve as spacers with the provisos that if an duplex arm segment has a length lower than 10 nucleotides the targeting domain is attached to the terminus of the arm domain which forms part of the terminus of the duplex arm segment.

In some embodiments for example, a region of the duplex arm segment of a DNA condensate monomer can have a sequence specific for a target DNA binding protein. In those embodiment the targeting domain is “embedded into the arm of the nucleic acid condensate can be used to specifically, bind, detect separate and/or purify the target DNA binding proteins, without addition of additional ligand moiety or aptamer sequence to the DNA condensate monomer Accordingly, functionalization of a nucleic acid condensate monomer can be performed by insertion of a DNA (or RNA) protein binding sequence into a duplex arm segment of the condensate monomer (e.g. within a nanostar structure)

In the schematic illustration of FIGS. 2A to 2B the targeting domain is located on the same single strand forming the condensate monomer (FIG. 2A) or four strands (FIG. 2B) forming the forming the condensate monomer, each of the strand presenting the targeting domain also present an interaction domain at opposite end of the same strand, and further presenting an additional interaction domain at a terminus of the nucleic acid strand complementary to the strand presenting the targeting domain.

Additional configuration of targeting domain with respect to the interactions domain of a functionalized condensate monomer of the disclosure are possible as will be understood by a skilled person. In some embodiments, the interaction and targeting domains of a nucleotide condensate monomer such as a nanostar can appear on an arm next to each other. For example, in a four arm nanostar, typically 3 arms can carry just the interaction domain, and the fourth arm can carry both an interaction domain, and a targeting domain adjacent to it a will be understood by a skilled person.

In some embodiments, the arm domain of the nucleic acid strands forming a nucleic acid condensate monomer range from 10 to 50 nucleotide in lengths

In some embodiments, the arm domain of the nucleic acid strands forming a nucleic acid condensate monomer range from 10 to 25 nucleotides in lengths.

In some embodiments, the interaction domain of the nucleic acid strands forming a nucleic acid condensate monomer is formed by single stranded “sticky end” range from 1 to 10 nucleotides in length.

In some embodiments, the interaction domain of the nucleic acid strands forming a nucleic acid condensate monomer is formed by single stranded loop range loop range from 4 to 40 nucleotides in total length, 4-15 nucleotide are binding regions presented for interaction, and 0-25 nucleotide flexible regions as will be understood by a skilled person

In some embodiments, the interaction domain of the nucleic acid strands forming a nucleic acid condensate monomer formed by a single nucleotide duplex structure [13]) and this would make condensates with a blunt interaction domain.

In some embodiments where the nucleic acid condensate monomer carries a blunt end interaction domain, and the blunt end has a 3′ G and a 5′ C.

In some embodiments where the functionalized nucleic acid condensate includes flexible linker domains the related sequences can preferably be AA or TT.

In some preferred embodiments, functionalized nucleic acid condensate of the disclosure can attach and present on a targeting domain an aptamer or an aptamer switch,

In some preferred embodiments, the ligand being an aptamer switch, the aptamer switch becomes fluorescent or more strongly fluorescent upon binding the target in a manner that provides for the detection and/or quantification of the target when the target binds, via quantification of the fluorescence of the aptamer.

In some preferred embodiments, the ligand being a fluorescent light-up aptamer, the aptamer becoming fluorescent or more strongly fluorescent upon binding a small molecule target fluorogen in a manner that provides for the detection and/or quantification of the target when the target binds, via quantification of the fluorescence of the light-up aptamer.

In some preferred embodiments, functionalized nucleic acid condensate of the disclosure can present on the targeting domain covalently attach a small molecule hapten.

In some preferred embodiments, functionalized nucleic acid condensate of the disclosure can present a single stranded nucleic acid aptamer covalently attached to a terminus of a duplex arm domain to form a nucleic acid extension,

In some preferred embodiments, functionalized nucleic acid condensate of the disclosure can present an antibody, nanobody, DARPin or other protein-based protein-binding entity covalently attached to a targeting domains.

In some preferred embodiments, functionalized nucleic acid condensate of the disclosure can comprise or attach to a targeting domain a double stranded DNA or RNA protein binding domain

In some embodiments, the ligand can be a small molecule, a peptide, a protein, a designed ankyrin repeat protein (DARPin), an antibody, a nanobody, a nucleic acid aptamer or a nucleic acid analog.

For example in some embodiments the ligand can be a small molecule such as those described in Tabana, 2023 #40} including biotin, encephalagen, aminopurvalanol, BRD0476, ssMP C11, melanogenin, sulfonyl amidine, TWS119, diminutol, GAPDS, quinostatin, SC1, QS11, KL001, withaferin, stauprimide, epolactaene, chromeceptin, myoseverin, PNRI-299, BMS-790052, ICG-001, 5-epi-sinuleptolide, resveratrol, bithionol, syrosingopine, rapamycin, nitazoxanide, ellagic acid, betulinic acid, gephyronic acid, axitinib, salinomycin, cryptotanshinone, FK506 and arctigenin

In other exemplary embodiments, the ligand can be a DARPin such as those that bind to human caspases and membrane transporter AcrB as described in “[14]

In further exemplary embodiments, the ligand can be a nanobody such as those that target SARS-CoV-2 as described in Mast et al., 2021 [15]

In additional exemplary embodiments, the ligand can be a nucleic acid aptamer as described in Pendergrast et al., 2005 and may include those that have specific binding to small molecules, peptides, proteins, growth factors, enzymes, immunoglobulins and receptors.

In yet another exemplary embodiment, the ligand can be a nucleic acid analog such as an antisense oligonucleotide as described in Wang wet al., 2022, [17] and may include those that bind to target messenger RNA or gene domains.

In each embodiments of a functionalized nucleic acid condensate monomers, a specific sequence and configuration of the nucleic acid monomer can be identified based on the specific target ligand combination °

For example In one embodiment, the target is a small molecule and the binding temperature is between 2-65° C., preferred 15-40° C., most preferred 20-37° C. Salt conditions are 50 mM-1M NaCl, preferred 100-150 mM, most preferred 150 mM. In the case where neither ligand nor small molecule target are sensitive to thermal degradation, the upper temperature limit can be extended to 100° C., or to around 80° C. In the case where the ligand-target binding interaction is of the biotin-streptavidin type, the maximum temperature should not exceed 70 C, and the salt concentration should not exceed 200 mM NaCl.

In another embodiment, the target and/or ligand is an antibody, and the binding temperature is between 20-37° C. and the salt concentration is 100-200 mM NaCl.

In yet another embodiment, the ligand is a nanobody, and the binding temperature and salt conditions are limited by the stability of the underlying nucleic acid condensate.

In another embodiment, the ligand is an aptamer and the binding temperature is between 20-37° C. and the salt concentration is between 50-200 mM NaCl, preferred 25-37° C. and 100-150 mM NaCl.

In some embodiments functionalized nucleic acid condensate monomers of the disclosure and can be included in a set of nucleic acid condensate monomers optionally comprising unfunctionalized nucleic acid monomers to form a functionalized set of nucleic acid condensate monomers.

In embodiments herein described a functionalized set of condensate monomers comprises functionalized condensate monomers configured to specifically bind a biomolecular target, and to form, in aqueous solvent and under condensing thermodynamic conditions, a functionalized nucleic acid condensate having a distinct nucleic acid condensate density via liquid-liquid phase separation (LLPS).

In the functionalized nucleic acid condensate the nucleic acid condensate is triggered by the intermolecular interaction of interaction domains of functionalized and optionally also unfunctionalized condensate monomers and includes a structural framework formed by the scaffold moieties of duplex arm segments of functionalized and optionally unfunctionalized condensate monomers forming part of the condensate.

In the functionalized set of nucleic acid condensate monomers, at least one nucleic acid monomer is a functionalized nucleic acid monomers of the present disclosure presenting a ligand configured to specifically bind the biomolecular target at the condensing thermodynamic conditions

In some embodiments, the functionalized set of nucleic acid condensate monomers comprises an unfunctionalized nucleic acid condensate monomer having a same structure of the functionalized condensate monomer with the exception the ligand In some embodiments, unfunctionalized monomers can comprise nucleic acid monomers having a same structure of the functionalized condensate monomer with the exception the ligand and of one or more of the length of arm domain interaction domain and or junction domain, as well number of duplex arm segments, and/or valency. In some embodiments unfunctionalized monomers can have any nucleic structure different from the structure of the functionalized nucleic acid condensate (e.g. single strand sticker-spacer configuration) as long as their configuration allows intermolecular interaction with the interaction domains of the functionalized nucleic acid monomers of the set, to form a functionalized condensate in the sense of the disclosure.

In some embodiments, the composition of functionalized and unfunctionalized condensate monomers in the set of functionalized condensate monomers can be selected based on the biomolecular target to include different percentages of functionalized and unfunctionalized condensate monomers to adjust for molecular weight, dimension and other features that can affect the condensing thermodynamic conditions and change the condensate properties to affect the related structural integrity (e.g. by lowering the melting temperature) or affect layering by changing condensate density. In those embodiments, a certain percentage of the nucleotide monomers of the functionalized set of nucleotide monomers can be formed by unfunctionalized monomers. to correct the thermodynamic conditions. For example in a functionalized set of condensate monomers, can be formed by 4 arm nanostars without the targeting domain forming up to 95% of the stars, and 4 arm nanostars that include the targeting domain forming 5% or more to accommodate a biomolecular target of high dimension. Such an approach can used whenever the molecular weight of the target approaches that of the condensate monomers themselves. In some embodiments, the fraction of “functionalized condensate monomers of the set can be adjusted so that the condensate properties are preserved. In some embodiments the fraction of functionalized monomers is adjusted simply to avoid using too many costly ligands, more than is required. For example, in a condensate that is at 100 uM nanostars, the fraction of nanostars functionalized by a costly antibody might be only 10% because 10 uM of the antibody would be sufficient to bind all desired target molecules.

In some embodiments, the target is at such a low concentration, and such a low volume fraction of the condensate, that it does not perturb the condensate properties in the course of condensate assembly and layering. For example, bacterial cells at a relevant concentration of 100,000 CFU/mL would have a volume fraction of the condensate of 1E-9, and a mean separation of 0.2 mm, which would have an insignificant effect on condensate properties.

In some preferred embodiment the functionalized set of nucleic acid condensate monomers comprises a same condensate monomer functionalized and optionally also unfunctionalized and presenting self-interacting interaction domains. This functionalized set of nucleic acid monomers forms a nucleic acid condensate through interaction of a single self-complementary interaction domain presented on each monomer and mediating the formation of a corresponding functionalized nucleic acid condensate of the disclosure.

In some preferred embodiment the functionalized set of nucleic acid condensate monomers comprises two distinct nucleic acid condensate monomers functionalized and optionally unfunctionalized presenting a pair of complementary interaction domains, one of which occurs on a first nanostar, and one of which occurs on the second nanostar, and whose complementary binding interaction mediates the formation of the functionalized nucleic acid condensate.

In embodiments herein described functionalized set of monomers are engineered and placed under suitable thermodynamic conditions to form a nucleic acid condensate in the sense of the disclosure functionalized with a ligand configured to specifically bind one or more biomolecular targets

In particular in a functionalizes nucleic acid condensate of the disclosure a biomolecular target can be captured through binding of the ligand to the target domain of corresponding functionalized nucleic acid monomers comprised in the condensate the in accordance with the indication of the present disclosure (see e.g. Example 6).

In embodiments of the present disclosure a functionalized nucleic acid condensate has a distinct functionalized nucleic acid condensate density and comprises a set of functionalized nucleic acid condensate monomers herein described each configured to specifically bind a biomolecular target. In preferred embodiments the functionalized nucleic acid condensate comprise one or more nucleic acid condensate monomers formed by a functionalized nucleic acid nanostar structure of the disclosure.

In some embodiments functionalized nucleic acid condensate of the disclosure has a set density and is separated from a diffuse layer typically places on top within a suitable container such as a test tube. In particular, when a set of functionalized nucleic acid monomer aggregates it forms It forms at least one both: (1) a concentrated liquid-like or gel-like phase that settles to the bottom of a test tube spontaneously or under light centrifugation, and (2) a diffuse layer (herein also indicated as dilute gas-like phase) on top (see e.g. Example 6) Concentrations of monomers in the concentrated phases are typically in the 30-1000 micromolar range. Concentrations of monomers in the diffuse layer are typically less than a few micromolar in concentration.

In some embodiments a single type of condensate monomer, with self-interactions, is used to generate a single layer of affinity material. This layer of affinity of material may or may not be combined with other orthogonal layers of affinity material into a multi-layer system. The term “orthogonal” as used herein with respect to condensate layer and in general liquid phases, indicates layers are immiscible to the degree that they are visibly distinct and exhibit a boundary that enables them to be separated by pipetting.

In some embodiments more than one type of condensate monomer, is used to generate a single layer of affinity material. In those embodiments, the monomers do not have strong self-interactions, and a binary, tertiary, or higher mixture of condensate monomers can be required to generate the layer of affinity material for a condensate layered structured of the disclosure. In some embodiments, this layer of affinity of material can be combined with other orthogonal layers of affinity material into a multi-layer system.

In some embodiments the nucleic acid condensate monomers comprises a single strand of nucleic acid. Multiple subsections of the single stranded condensate monomers have interactions that mediate the interactions between condensate monomers.

In some embodiments the nucleic acid condensate monomers comprise a multistranded noncovalent complex with multiple double-stranded arms having sticky ended overhangs that mediate the interactions between condensate monomers, herein also identified as nanostars. In such embodiments up to at least twelve double stranded arms can connect in a nucleic acid flexible linker.

In some embodiments the nucleic acid condensate monomers comprise a multistranded covalent complex with single stranded arms having complementary overlaps that mediate the interactions between condensate monomers. In such embodiments a multi-armed small molecule can be used as the center of the condensate monomer, and the single-stranded arms of the condensate monomer are attached covalently to the small molecule. In those embodiments, up to at least six single stranded arms can connect to a single small molecule center (e.g., a polyethylene glycol star)

In some embodiments the functionalized layered nucleic acid condensate structure comprises at least two a functionalized nucleic acid condensate herein described, each comprising a ligand specific for a different biomolecular target and preferably interaction domains orthogonal to one another as will be understood by a skilled person

In embodiments herein described. The nucleic acid comprising the targeting domain with appropriate ligand can be added to a mixture of other nucleic acids at a relative concentration of 0.001%-100 depending on the one or more targets to be captured in the condensate. A skilled person will understand that structural features of the biomolecular target and their impact on the stability and organization of condensates. Accordingly, the proportion of the functionalized monomers binding the target vs unfunctionalized condensate monomers can be adjusted to reduce or increase the concentration of captured target in order to maintain the stability of the condensate, as would be understood by a skilled person upon reading of the present disclosure.

A functionalized nucleic acid condensate can be formed following contacting the functionalized monomers with the biomolecular target, e.g. within a and for a time and under condition allowing binding of the target to the ligand of the functionalized monomers, and then placing the resulting mixture under condensing thermodynamic conditions through suitable methods described here such as heating to an appropriate temperature followed by controlled cooling. The condensing conditions can be selected so that the condensate is liquid-like enough that it readily phase separates from the mixture (e.g. a bulk sample cleanly (either via thermal annealing (e.g. 50 C to 20 C over 1 hour), light centrifugation (e.g. 10 minutes at 10,000 g), or both, with a clear interface demarking the condensate and the diffuse phase.

The condensing thermodynamic conditions and the configuration and sequences of the monomers forming the condensate is selected based on the conditions allowing binding of the target with the ligand presented on the monomers. As will be appreciated by the skilled person, in certain cases, such as for a small molecule or nucleic acid aptamer ligand, the ligand is not degraded by exposure to an elevated temperature required to melt the nucleic acid strand mixture before forming the condensate (e.g., 40° C. or above), and the condensate can be formed by a variety of methods where careful control of this maximum temperature is not required. In other embodiments, such as when the ligand is a protein or an antibody that can be degraded by exposure to elevated temperatures, formation of the condensate at lower temperatures may be required (e.g., 37° C. or below). This result can be achieved, for example, by selection of a nucleic acid mixture that melts and forms a condensate at these lower temperatures (for example, by employing complementary associations between the interaction domains) and/or by annealing with increasing concentrations of salt at ambient temperature.

In embodiments, herein described once the target has been bound by the ligand in a targeting domain of the condensate, several method can be used to release and isolate the target.

In some embodiments, the individual layer may be removed from a multilayer condensate structure and a release agent can be added to the isolated layer; this release agent can be specific to disrupt only the binding between the ligand and the target, or can disrupt the structure in a non-specific manner to release the target as part of a general degradation of the structure.

In some embodiments, biomolecular targets captured in functionalized nucleic acid condensate of the disclosure is released in a controlled fashion.

For example one or more biomolecular target and components herein described can be separated by nucleic acid displacement or enzyme-assisted release . . . .

The term “displacement”, “strand displacement reaction”, or “branch migration reaction” as used herein generally indicates the process in which two polynucleotide strands with partially or full complementarity hybridize, displacing in the process one or more pre-hybridized strand or sequence. The strand displacement process can be experimentally tested or measured according to techniques that are identifiable by a skilled person.

In some embodiments the complementary binding between a release polynucleotide and selected region of the targeting segment with the ligand bound to the biomolecular target can be at least as stable, and possible more stable, than the complementarily binding between internal regions within the targeting segment. In some embodiments mismatches within selected regions within the targeting domain and/or stabilizing modifications such as 2′-O-methyls can be localized in selected regions of the targeting segment, to drive and control the release of the ligand and biomolecular target from the nucleic acid condensate. In determining the configuration, length, and sequence, the release conditions can also be considered (e.g., temperature and salts concentrations).

In particular, in embodiment where a nucleic acid is used as a ligand for a target nucleic acid, recovery is simply a matter of applying a strand that is complementary to the ligand, potentially using an additional nucleic acid toehold, to speed kinetics of the resulting strand displacement reaction. Accordingly, in embodiments wherein that nucleic acid aptamers are used as the affinity reagent for a small molecule or protein, recovery can be performed by applying a strand that is complementary to the aptamer, potentially using an additional nucleic acid toehold to speed kinetics of the resulting kleptamer displacement reaction. In the case of a multiplexed separation, nucleic acids used to release different targets can be added in a stepwise fashion, so that different targets can be released from their corresponding layers in any desired order.

In embodiments herein described, biomolecular targets can be released singly, or in groups, as programmed by the order of addition of the release agents which can be formed by release polynucleotides and/or enzymes for enzyme assisted recovery.

The term “enzyme-assisted” as used herein is defined to mean any chemical process where a protein or other chemical entity is used to catalyze or increase the rate of a chemical reaction. The protein used for this purpose can comprise chains of amino acids (natural or unnatural), that may or may not contain other chemical variations and can have a defined secondary structure. Typically, enzymes catalyze reactions through binding to specific or non-specific target molecular portions usually indicated as binding sites.

The chemical reaction in embodiments, comprise reactions between nucleic acid of the targeting domain and ligand attached thereto and/or reactions between ligand of a nucleic acid condensate monomer and corresponding biomolecular target as will be understood by a skilled person.

In particular in some embodiments, the enzyme assisted release of a biomolecular target is directed to release a biomolecular target from a targeting domain within a desired environment,

In some embodiments, the target can be removed from the mixture of condensate layers by addition of a specific release agent that disrupts only the binding between the ligand and the target. The target can then diffuse into the dilute top layer and be removed. In the case where multiple targets are bound by different targeting domains and/or in different layers, the release of a specific target may be programmed by addition of a specific release agent.

In some embodiments where the ligand is a small molecule such as biotin, a suitable release method can include exposure of the condensate to a denaturing buffer, such as a solution containing sodium dodecyl sulfate and a high temperature (95-100° C.).

In some embodiments where the ligand is an antibody or a nanobody, the target can be released by exposure to a suitable antigen with a higher specificity for the antibody than the target.

In some embodiments where the ligand comprises a nucleic acid (such as an aptamer or nucleic acid analog) the target can be released by exposure to a release polynucleotide. Such a polynucleotide may comprise RNA or DNA and can feature a “toe-hold” oligonucleotide region that is 5 or more nucleotides longer than the aptamer interaction domain within the condensate to aid in the target release. The release polynucleotide may be designed to have specific affinity for the interaction domain of a particular aptamer in the condensate mixture, allowing for release of a specific target, or may be designed to disrupt the binding of the condensate in general, allowing for general target release.

In the case where it desired to degrade the condensate structure to release the target, a suitable method can be to expose the condensate to deoxyribonuclease (DNase) enzyme, which may hydrolyze the underlying nucleic acid structure while leaving other bonds intact such as those in the target (e.g., if the target is a small molecule or protein)

In the case where the ligand is an antibody or a nanobody, the target may be released by exposure to a suitable antigen.

In the case where the ligand comprises a nucleic acid (such as an aptamer or nucleic acid analog) the target may be released by exposure to a release polynucleotide. Such a polynucleotide may comprise RNA or DNA and may feature a “toe-hold” oligonucleotide region that is 5 or more nucleotides longer than the aptamer interaction domain within the condensate to aid in the target release. The release polynucleotide may be designed to have specific affinity for the interaction domain of a particular aptamer in the condensate mixture, allowing for release of a specific target, or may be designed to disrupt the binding of the condensate in general, allowing for general target release.

In the case where it desired to degrade the condensate structure to release the target, a suitable method may be to expose the condensate to deoxyribonuclease (DNase) enzyme, which may hydrolyze the underlying nucleic acid structure while leaving other bonds intact such as those in the target (e.g., if the target is a small molecule or protein)

In some embodiments, one or more functionalized nucleic acid condensate can be included in a functionalized layered nucleic acid condensate structure, comprising: an optional diffuse phase layer and at least one functionalized nucleic acid condensate layer of the disclosure, each layer having a distinct density. In the nucleic acid condensate structure, the optional diffuse phase layer and the at least one functionalized nucleic acid condensate layer are arranged in the structure, in order of increasing density, with the densest layer positioned opposite the least dense layer within the structure the optional diffuse layer being the least dense layer in the structure (see Example 4)

In the nucleic acid condensate structure, at least one nucleic acid condensate layer comprises a functionalized nucleic acid condensate herein described comprising at least one functionalized nucleic acid condensate attaching a ligand specific for a biomolecular target, in a configuration in which the ligand is presented for binding to the biomolecular target when present, the binding facilitating selective capturing of the target compound within the nucleic acid condensate layer containing the ligand.”

In some embodiments, functionalized layered nucleic acid condensate structure omits a diffuse layer. In those embodiments, for each condensate, there exists a high condensate monomer concentration (e.g. 1 mM nanostars above which a an environment will be all condensate and will not segregate into a liquid and a diffuse layer which can be identified by a skilled person based on the condensing thermodynamic conditions. If such a state is desired, a method to provide the corresponding structure can comprise provide a condensate or condensate structure comprising a diffuse layer and one or more liquid state layer, and then remove the diffuse layer. In those embodiment, the remaining condensate can be used by itself or in combination with other condensates and a diffuse layer will not form upon reannealing or further centrifugation. This configuration of the functionalized layered nucleic acid condensate structure of the disclosure can be preferred in methods of the disclosure where the functionalized layered nucleic acid condensate structure is used for separation of molecular targets as it will be understood by a skilled person upon reading of the present disclosure.

In a functionalized layered nucleic acid condensate structure, each condensate layer of the structure is formed by a distinct functionalized sets of condensate monomers configured to form condensate separate with a clearly defined interface from other condensates. Without a clearly defined interface or interfaces then molecules cannot be separated or reliably detected or separated.

In a functionalized layered nucleic acid condensate structure, a clearly defined interface is obtained by selecting interaction domains of the distinct functionalized sets of monomer forming each condensate layer of the structure are orthogonal and preferably highly orthogonal so that well defined “crisp” interfaces between layers are maintained. There are many approaches to perform this selection identifiable by a skilled person For example in some embodiments an approach can be used based on hamming distance [18]). In particular, in some embodiments an approach can be used that selects for short sticky ends of length 6, Hamming distance of 4 or greater between two interaction domains are preferred. Alternatively thermodynamic design of interaction domains can be used.

In some embodiments orthogonality between interactions domains of distinct functionalized sets of monomer forming each condensate layer of the structure can be obtained by selecting different interaction domains (e.g. a blunt interaction domain for functionalized sets of monomer forming a condensate layer, and sticky end interaction domain for functionalized sets of monomer forming a distinct condensate layer of the structure).

In a functionalized layered nucleic acid condensate structure targeting domains for different biomolecular targets within a same or different functionalized sets of nucleic acid condensate monomers are orthogonal and preferably highly orthogonal.

In embodiments where the biomolecular targets are nucleic acids orthogonality of the targeting domain is fairly easily engineered using Hamming distance or thermodynamics as above; for proteins, targeting domains is tested for orthogonality with the different biomolecular targets of interest. For example by engineering targeting domains so that they are orthogonal relative to protein targets avoiding sequences have too much sequence homology, to their corresponding targets. Thus as a quantitative starting point, a skilled person can use different protein-protein binding targeting domains if the targeting domains share no more than 30% sequence identity, and the biomolecular targets share no more than 30% sequence identity.)

Formation of the condensate and binding of one or more biomolecular targets can be detected with techniques such as light absorption and/or fluorescence techniques.

For example, the purity of a nucleic acid condensate can be assessed by comparing the ratio of absorbances at 260 nm and 280 nm, as will be appreciated by those skilled in the art. In general, a ratio Abs₂₆₀/Abs₂₈₀>1.8 may be considered “pure” for condensates comprising DNA, and a ratio Abs₂₆₀/Abs₂₈₀ ratio >2.0 may be considered “pure” for condensates comprising RNA.

Both absorption and fluorescence methods described above can also be performed in situ using a light source of suitable resolution that can excite and detect an individual condensate without affecting adjacent layers. For example, each layer of a multilayer condensate assembly in a cuvette may be interrogated as part of a fluorescence assay using a tunable laser that can be focused on an individual layer. Quantification of the emission intensity for an individual layer using a calibration curve gives the condensate layer density as described above; this may be repeated for an adjacent layer by moving the focus of the laser to the next layer.

In another exemplary method, ˜100 uL-2 mL of condensate layer can be removed and weighed using a microbalance, and the density can be obtained directly by dividing the mass of the sample by its volume. This may be achieved in practice, for individual layers or a mixture of layers, by placing a sample of the layer or the mixture of layers in a suitable capillary tube of known mass and volume. The capillary tube can be broken at an observed boundary between layers, and the subsequent individual capillary fragments weighed and their lengths determined. After correction for the mass of the capillary material, the density of a particular layer may be obtained by dividing the mass of the layer material by the volume of capillary fragment occupied, as determined from the measured length of the capillary segment. Furthermore, if large samples are available (e.g., >10 mL), measurement of the nucleic acid condensate layer viscosity using a Ubbelohde viscometer and comparison to known nucleic acid standard viscosities provides the condensate concentration.

In a functionalized nucleic acid condensate, the nucleic acid monomers are designed to have binding sites for one or more target molecule. These binding sites can be implemented with small molecule ligands, antibodies, aptamers, or any other class of affinity reagent that can be chemically coupled to the nucleic acid monomers. Target molecules are partitioned into the nucleic acid condensate phase, and other components of the complex mixture are excluded in the supernatant. The nucleic acid condensate phase can be washed to remove contaminants. Following washing, the target molecules can be released from the nucleic acid condensate phase using traditional release methods, or in the case that the ligands are nucleic acid aptamers, the target molecules can be released using a highly specific nucleic acid displacement reaction, known as a kleptamer reaction. Once released, the target molecules can be easily separated from the nucleic acid condensate.

Accordingly in some embodiments, the functionalized layered nucleic acid condensate structure comprises one or more nucleic acid condensates wherein the one or more condensates are visibly distinct from a dilute gas-like phase and with each other, the one or more condensates contain ligands which function as binding domains for one or more target molecules or cells

In some embodiments, in the functionalized layered nucleic acid condensate structure, one or more condensates comprise layers in a macroscopic system, which provides for the separation of the target molecules.

In some embodiments, in the functionalized layered nucleic acid condensate structure, the nucleic acid condensates are comprised of monomers themselves comprising multi-stranded DNA complexes with up to twelve double-stranded arms, wherein at least one of the double-stranded arms has a sticky end.

In some embodiments, in the functionalized layered nucleic acid condensate structure, the nucleic acid condensates are comprised of monomers themselves comprised of up to six multiple single strands, covalently bound to a multivalent small molecule linker.

In some embodiments, in the functionalized layered nucleic acid condensate structure, the nucleic acid condensates are comprised of monomers themselves comprised of single DNA strands with multiple binding domains concatenated along their length.

In some embodiments, in the functionalized layered nucleic acid condensate structure, the ligand binding domains for the target molecules are small molecules, aptamers, antibodies, DARPins, nanobodies, lectins, or mixtures thereof.

In some embodiments, in the functionalized layered nucleic acid condensate structure, the ligands are aptamers for blood factors.

In some embodiments, in the functionalized layered nucleic acid condensate structure, the ligands are aptamers for the Fc portion of an antibody.

In some embodiments, in the functionalized layered nucleic acid condensate structure, the ligands are specific to the IgGs of a particular animal. In some of those embodiments there are three visibly distinct condensates specific to three different animal IgGs.

In some embodiments, in the functionalized layered nucleic acid condensate structure, the ligands bind protein expression tags.

In some embodiments, in the functionalized layered nucleic acid condensate structure, there are three visibly distinct condensates specific to three different protein expression tags. In some of those embodiment the ligand fluoresces upon binding its cognate protein expression tag.

In some embodiments, in the functionalized layered nucleic acid condensate structure, the ligands bind a specific bacterial strain, a viral antigen, polyadenylated mRNAs, or a CAR T cell.

In some embodiments, in the functionalized layered nucleic acid condensate structure, the layers have been formed by centrifugation or through addition of salt or via thermal incubation or annealing.

In some embodiments, in the functionalized layered nucleic acid condensate structure, the condensate monomers in different layers have different arm lengths, have different sticky end strengths, or have different numbers of arms.

In some embodiments, in the functionalized layered nucleic acid condensate structure, the density of condensate is controlled by a combination of the number of arms and the fraction of these arms which have a blunt end as will be understood by a skilled person upon reading of the present disclosure.

In some embodiments, the functionalized layered nucleic acid condensate structure, comprises two condensates, a first condensate having monomers with four arms, each arm having a sticky end, and a second condensate having monomers with six arms, where four arms have sticky ends and two arms have blunt ends.

In some embodiments, in the functionalized layered nucleic acid condensate structure, the density of the condensate is controlled by the fraction of nanostars of different arm numbers, and where the number of arms bearing sticky ends within a single condensate is constant. The density of each layer and the condensate layering in this case is driven by the average molecular weight of the monomers, as determined by average number of arms on the condensates within each layer, which within a layer is in turn controlled by the fraction of nanostars having each arm number, and which can be calculated by a weighted sum.

In some embodiments, the functionalized layered nucleic acid condensate structure comprises three condensates, a first condensate having four arms, each arm having a sticky end, and a second condensate containing a mixture of 4-armed monomers and 8-armed monomers, where the 4-armed monomers have a sticky end on each arm, and the 8-armed monomers each have 4 sticky ends and 4 blunt ends, and a third condensate having 8-armed monomers, where each 8-armed monomer has 4 sticky ends and 4 blunt ends. The density of each layer and the condensate layering in this case is driven by the average molecular weight of the monomers, as determined by average number of arms on the condensates within each layer. For example if the second condensate is a 50-50 mix, its average number of arms will be 6 and the average number of arms for each layer will be 4, 6, and 8 respectively, as will be understood by a skilled person upon reading of the present disclosure.

In some embodiments, in the functionalized layered nucleic acid condensate structure, the ligand is an aptamer for streptavidin.

In some embodiments, in the functionalized layered nucleic acid condensate structure, captured streptavidin can be released from the condensate using a complementary strand to the aptamer via a kleptamer reaction.

In some embodiments, in the functionalized layered nucleic acid condensate structure, there are three different 4-arm condensate monomers, having arm lengths of 15, 20, and 25 respectively, wherein upon centrifugation the resulting condensates layer, bottom to top, in order of the shortest arm length to the longest arm length. In particular in some of those embodiments the sequences of the condensate monomers can be:

For the 15-nt arm length:
SEQ ID NO 1:
GTAGTCTACAGTGCCAACTGGGCAGAATTCCCAGCTAGC;
SEQ ID NO 2:
GGGAATTCTGCCCAGAACGTCACCAGAAGCACAGCTAGC;
SEQ ID NO 3:
GTGCTTCTGGTGACGAAGACGGAATCTCCGTCAGCTAGC;
SEQ ID NO 4:
GACGGAGATTCCGTCAAGGCACTGTAGACTACAGCTAGC
For the 20-nt arm length:
SEQ ID NO 5:
CTACTATGGCGGGTGATAAAAACGGGAAGAGCATGCCCATCC
ACGATCG;
SEQ ID NO 6:
GGATGGGCATGCTCTTCCCGAACTCAACTGCCTGGTGATACG
ACGATCG;
SEQ ID NO 7:
CGTATCACCAGGCAGTTGAGAACATGCGAGGGTCCAATACCG
ACGATCG;
SEQ ID NO 8:
CGGTATTGGACCCTCGCATGAATTTATCACCCGCCATAGTAG
ACGATCG
For the 25-nt arm length:
SEQ ID NO 9:
CGCTACAATACAGTTACAAGAATGCAACGCTTGATGTATGCA
CGTATGTTGCACACGTG;
SEQ ID NO 10:
GCAACATACGTGCATACATCAAGCGAACATATCTCATATTCG
TGCCACTATGACACGTG;
SEQ ID NO 11:
CATAGTGGCACGAATATGAGATATGAACAGTAGGGCAGCAAAG
ACTACGGTGACACGTG;
SEQ ID NO 12:
CACCGTAGTCTTTGCTGCCCTACTGAAGCATTCTTGTAACTGT
ATTGTAGCGACACGTG

In some embodiments, functionalized nucleic acid condensate and functionalized layered nucleic acid condensate structure herein described can be used to separate one or more biomolecular targets from a mixture.

The term “separate” or “separation” in the context of polynucleotide condensates refers to the process of isolating and extracting specific target molecules from a mixture through their selective recruitment into nucleic acid condensates. This method leverages the ability of engineered nucleic acid sequences to recognize and concentrate desired targets within condensed phases, effectively purifying them from the original mixture. The degree of separation is typically quantified by the partition ratio, which measures the concentration of the target molecule inside the condensate compared to its concentration in the surrounding solution as will be understood by a skilled person.

Exemplary mixtures in the sense of the disclosure comprise processed or unprocessed samples of an environment provided for use in testing, examination, or study. The environment can comprise a biological environment including living beings and, in particular, human beings.

The term “sample” as used herein indicates a limited quantity of something that is indicative of a larger quantity of that something, including but not limited to fluids from the biological environment, such as tissues, organs or other biological material from the living being such as urethra, urine, cervix, vagina, rectum, oropharynges, conjunctiva, or any body fluids, cultures, tissues, commercial recombinant proteins, synthetic compounds or portions thereof. Exemplary biological samples comprise: cheek tissue, whole blood, dried blood spots, organ tissue, plasma, urine, mucus, mucosal secretions, vaginal fluids and secretions, urethral fluids and secretions, feces, skin, hair, or tumor cells, among others identifiable by a skilled person. Biological samples can be obtained using sterile techniques or non-sterile techniques, as appropriate for the sample type, as identifiable by persons skilled in the art. Some biological samples can be obtained by contacting a swab with a surface on a human body and removing some material from said surface, examples include throat swab, urethral swab, oropharyngeal swab, cervical swab, vaginal swab, genital swab, anal swab.

Typically, a biological sample provided for use in testing, examination, or study is further processed with agents which are selected to allow and/or facilitate the intended testing examination or study. Exemplary agents comprise a buffer agent which is a chemical compound that is capable of maintain the pH value stability of an aqueous solution, or a chaotropic agent which a molecule in water solution that can disrupt the hydrogen bonding network between water molecules and can be used to disrupt membrane integrity of a cell. Additional agents used to treat a biological sample comprise a biological medium, an antibiotic, and additional agents identifiable by a skilled person in view of the intended use of the biological sample. Depending on the type of biological sample and the intended analysis, biological samples can be used freshly for sample preparation and analysis, stored at room temperature, stored under refrigeration, stored frozen, treated with a lysis solution and then stored, or fixed using fixative. For example, urine can be mixed with specimen transport and storage tube (see e.g. Aptima® Urine Specimen Transport Tube and additional commercially available containers).

A separation in the sense of the disclosure can be performed for preparative purposes, and therefore be directed to prepare fractions of the mixture components, typically the nucleic acid, that can be saved and/or used to perform additional reactions.

In particular, in embodiments of the disclosure the separation can be performed to detect and/or purify the target compound from according to an affinity based like chromatographic approach.

In affinity-based chromatography, a binding partner for the target is typically immobilized on a stationary phase, typically a solid support such as a particulate resin which can be packed in a column or magnetic beads that can be pulled to the bottom or side of a test tube. Binding partners are also commonly termed ligands, or affinity reagents on the solid support are also part of the stationery phased and are also indicated as the “affinity material” which is contacted with a mobile phase comprising the mixture to be separated.

In embodiments herein described the separation is performed with an approach where the stationary phase is replaced by functionalized nucleic acid condensate and related layered structures in accordance with the disclosure, which is used to contact the mobile phase of the mixture to perform separation.

In some embodiments, comprises providing a set of functionalized nucleic acid condensate monomer herein described functionalized with a ligand specific for the biomolecular target.

The method further comprises contacting the set of functionalized nucleic acid condensate monomers with the mixture to allow binding of the biomolecular target with the ligand, and. following the contacting with the mixture, inducing condensation of the set of functionalized nucleic acid condensate monomers to form a functionalized nucleic acid condensate specifically binding the biomolecular target.

The method also comprises layering of the functionalized nucleic acid condensate in nucleic acid condensate layer within a functionalized layered nucleic acid condensate structure of the disclosure; and releasing the biomolecular target from the ligand of the functionalized nucleic acid condensate specifically binding the biomolecular target.

In some embodiments, the releasing provides a released biomolecular target within the functionalized nucleic acid condensate layer where the biomolecular target was specifically bound.

In some embodiments the releasing provides a released biomolecular target located in a layer of functionalized nucleic acid condensate layer structure other than functionalized nucleic acid condensate layer where the biomolecular target was specifically bound, such as the diffuse layer or a capture layer of the layered nucleic acid condensate structure where the biomolecular target is allowed to move e.g. through diffusion.

In some embodiments, the releasing can be provided following removal of one or more layer from the nucleotide functionalized nucleic acid condensate layer structure and formation of a further functionalized nucleic acid condensate layer structure through mixing of the removed functionalized nucleic acid condensate layer with a fresh buffer and layering the resulting functionalized nucleic acid condensate layer (herein also “relayering’) in a relayered functionalized nucleic acid condensate layer structure, the diffuse layer comprising by the fresh buffer is herein indicated as a supernatant layer.

In some embodiments. method further comprises separating the released biomolecular target from the layer of the functionalized nucleic acid condensate layer structure where the released biomolecular target is located following the release of the biomolecular target from the functionalized condensate monomer specifically binding the target.

In some embodiments, the releasing can be performed by contacting a nucleic acid condensate layer of the layered nucleic acid condensate structure with a release agent configured to specifically release the biomolecular target from the ligand of the functionalized nucleic acid monomer to obtain the release of the biomolecular target from the nucleic acid condensate; and separating the released biomolecular target from the nucleic acid condensate layer.

In some embodiments, the contacting can be performed when then nucleic acid condensate layer is within the functionalized nucleic acid layered structure and the release can be performed by allowing diffusion of a released biomolecular target into a diffuse layer or a capture layer and the separating the biomolecular target from the diffuse layer or capture layer (e.g. by centrifugation).

In some embodiments, the contacting can be performed following removal of the nucleic acid condensate layer binding the biomolecular target from the nucleic acid layered structure to obtain release of the biomolecular target and related separation from the condensate layer, outside the nucleic acid condensate layered structure (e.g. separate container).

In some embodiments, the contacting can be performed when the nucleic acid condensate layer is within nucleic acid condensate layered structure to allow release into a capture layer, followed by removal of the nucleic acid capture layer from the nucleic acid layered structure to obtain release of the biomolecular target outside the nucleic acid condensate layered structure.

In some of embodiments, removal of a condensate layer binding the biomolecular target and/or removal of capture layer where the target is released, can be preceded by removal of the diffuse layer if present and/or other layers of the structure as will be understood by a skilled person upon reading of the present disclosure. The removed layers can be discarded in or in multiplexing embodiments placed in separate environment (e.g. a container) where the biomolecular target is released from the ligand and separated from the condensate layer.

In some embodiments, the functionalized nucleic acid layered structure is formed by a single layer with a diffuse layer and after a first layering, the original diffuse layer is removed and discarded, and the corresponding layer in the layered structure is and replaced with fresh buffer to form a supernatant layer. The release agent is then contacted with the remaining condensate layer, and after agitation and relayering, the target is now in the supernatant layer. In those embodiments, this supernatant layer can then be removed with the target in it. In the case of n layers, then each release agent for each particular layer is applied sequentially in order. After an application of the release agent for a particular layer, the tube is mixed and relayered, and the supernatant diffuse layer with the target for the particular layer can be removed.

In some embodiments, wherein the functionalized nucleic acid layered structure is formed by a single nucleic acid functionalized nucleic acid condensate layer specifically binding a biomolecular target, and comprising a diffuse layer, after a first layering, the layer with the targeting domain is removed from functionalized nucleic acid layered structure and placed in a separate environment (e, g, moved by pipetting into a fresh tube). Buffer with the release agent is added and mixed with the functionalized nucleic acid condensate layer specifically binding a biomolecular target which is then ‘relayered’ and results in release of the biomolecular target within a supernatant layer formed by the fresh buffer. The target is then collected from the resulting diffuse phase. In the case of n layers each specifically binding a distinct biomolecular target, then after the first layering, each condensate layer is pipetted into a different fresh tube, buffer with release agent is added, mixed and a second layering is performed for each tube (now with only 1 layer in it). The target is then collected from the diffuse phase.

In some embodiments where the biomolecular target is different from the nucleic acid forming the functionalized nucleic acid monomer specifically binding the target, the releasing can be performed by contacting the functionalized nucleic acid condensate layer comprising the released biomolecular target with an agent that destroys the condensate monomer (e.g. a nuclease like DNAse for a DNA condensate monomer, or an hydrolytic agent, e.g. base for RNA condensate monomers) which would release the biomolecular target be destroying the the functionalized nucleic acid monomer. Any agent that releases the target molecule in this way (via digestion) results in the conversion of the nucleic acid condensate monomers in a mixture of mononucleotide or small fragment. In some embodiments the resulting mixture of mononucleotide or small fragment can be separated after digestion from the target, and for many targets (e.g. proteins 5 kD or greater) this can be done via a simple 5 kD molecular weight cut off filter that allows the digested DNA mononucleotides or short fragments (5 nt or less) to flow through.

In some embodiments, where the releasing is performed following removal of a layer of the functionalized nucleic acid condensate structure, e.g. by one of the physical separation of the layering/diffuse layer strategies herein described, even if digestion is not used it can be desirable performing a separation step directed to remove contaminating nucleic acid left in the released target biomolecules (e.g. the released strand, or some nucleic acid condensate monomers). in those embodiments, the contaminating nucleic acid can be removed by digestion and filtering as in 4, or by selectively removing the nucleic acid condensate monomers using silica beads (here the flow through or supernatant from the silica beads would be taken, which is the opposite of the normal use of the silica which is to catch and retain nucleic acids).

In some embodiments, the functionalized nucleic acid monomers can be used makes affinity-based separation, purification, and detection of a biomolecular targets multiplexable.

In those embodiments at least two sets of functionalized nucleic acid condensate monomers herein described each set functionalized with one or more ligand specific for one or more of the at least two biomolecular targets, each of the at least two sets of nucleic acid condensate monomers configured to form a nucleic acid condensate layer having a distinct density

The method further comprises contacting the at least two sets of functionalized nucleic acid condensate monomers with the mixture to allow binding of the biomolecular target with the ligand, and. following the contacting, inducing condensation of the at least two sets of functionalized nucleic acid condensate monomers to form at least two nucleic acid condensates one for each set of monomers.

The method also comprises layering of the at least two functionalized nucleic acid condensates in at least two polynucleotide condensate layers within a layered nucleic acid condensate structure of the present disclosure; contacting the layered nucleic condensate structure with at least two release agents each configured to specifically release a biomolecular target from a corresponding ligand of the at least two sets of functionalized nucleic acid monomer to obtain the release of the at least biomolecular target from of the at least two sets of nucleic acid condensate monomers; and separating the released at least two biomolecular target from said the nucleic acid condensate layer.

In embodiments, herein described the two primary agents for enhancing LLPS are salt and crowding agents. Both will increase the temperature at which phase separation occurs. Monovalent salts such as sodium and potassium can be used for nucleic acid liquid condensate formation in the range of 100 mM to 1M depending on the number of arms, arm length, interaction domain strength and flexibility of the nanostar. Divalent cations such as magnesium can be used at a concentration that is 50× lower than monovalent salts (i.e. 2 mM to 20 mM). Molecular crowding agents for LLPS include but are not limited to: dextran, PEG, and BSA, See Collette et al

Accordingly, multiple affinity-based reagents can be used simultaneously to achieve multiplexed separation of multiple high value targets from a single mixture. This is achieved by designing multiple affinity-based reagents that separate spontaneously or under light centrifugation into layers within a single test tube as will be understood by a skilled person

In separation methods herein described separation of different nucleic acid condensates into different layers for multiplexed separation can be based either on different architectures for the condensate, or a different experimental protocol. In some embodiments separation into layers is based on the length of the condensate monomer arms, with shorter armed monomers creating layers that lie below monomers with longer arms. In some embodiments separation is based on the strength of the sticky ends with monomers having stronger sticky ends creating layers that lie below monomers with weaker sticky ends. In some embodiments, separation is based on the number of arms of the condensate monomer. In such embodiments some of the arms of the condensate monomers can be double stranded but have no binding domain to other condensate monomers. In some embodiments this is performed by selecting the ends of the arms of a condensate monomer to be blunt ends. In such embodiments a particular layer of condensate can be comprised of mixtures of monomers having different numbers of arms, where the fraction of monomers having a particular number of arms dictates the density and position of the particular layer of condensate.

In embodiments, where the separation is multiplexed separation of a plurality of biomolecular targets, the releasing of the distinct biomolecular target can be performed at a same or different time depending on whether the releasing step can be performed through approaches which result in orthogonal separation of the plurality of biomolecular targets. In those embodiments, the timing and selection of releasing methods is performed to minimize release of the plurality of biomolecular target in a mixture, unless such release is desired, as will be understood by a skilled person

Accordingly in some embodiments, where the separation is multiplexed separation of a plurality of biomolecular targets, the layers could be mechanically separated into fresh tubes via pipetting, followed by the addition of release strands specific to the tube into which the layers are pipetted. (addition of a release strand requires mixing and relayering into a single layer plus diffuse phase where the diffuse phase is collected). This approach would allow the different targets to be collected in a single parallel step operated on different tubes.

In some embodiments, where the separation is multiplexed separation of a plurality of biomolecular targets, the biomolecular targets can be collected serially, in a number of steps equal to the number of layers having biomolecular targets. In some of these embodiments, where the plurality of biomolecular target is specifically bound by nucleic acid aptamers ligand, release can be performed through strand displacement of the nucleic acid aptamer ligands specifically binding the biomolecular target. In those embodiments, a first release strand can contact the layered system (contacted, mixed, relayered) and the diffuse phase with a first biomolecular target collected, then a second release strand applied and the diffuse phase with a second biomolecular target collected and the steps can be performed as many times as required by the number of layers and targets in the functionalized nucleic acid condensate layered structure as will be understood by a skilled person.

In some embodiments, layering and/or relayering can be performed by centrifugation set up to created layers according to approaches identifiable by a skilled person. In some embodiments incubation at a particular temperature or a thermal anneal is used to create layers. In some embodiments layers spontaneously form upon addition of salt that initiates condensate formation.

In some embodiments, nucleic acid condensates occur as freely pipettable liquids. In other embodiments, nucleic acid condensates form hydrogels. In some embodiments stepwise release of different targets from different condensate layers may be employed. In some embodiments, multiple layers may be physically separated by pipetting before targets are released. In other embodiments, targets are released by DNA strand displacement, or kleptamer protein displacement and collected in the supernatant above a condensate. In some embodiments condensate layers may be selectively destroyed by DNA strands that disturb the interactions between condensate monomers, releasing monomer-target complexes into the supernatant where they can be collected. In such embodiments, monomer-target complexes may be broken in a separate purification step, to yield free targets.

In some embodiments the targets are RNA or DNA molecules, proteins, bacterial or eukaryotic cells. In some embodiments the targets are polyadenylated mRNAs, and the ligands are polyT extensions to the nucleic acid monomers, for the enrichment of expressed RNAs. In related embodiments polyA or polyA plus toehold strands are used to release transcripts from the nucleic acid condensate.

In some embodiments the ligands are for common protein purification tags, including but not limited to green fluorescent protein (GFP), streptavidin, polyhistidine tags, FLAG tags, chitin binding domain, maltose binding protein, horseradish peroxidase, alkaline phosphatase, T7 epitope, or galactose binding domain. In such embodiments, a multi-layer condensate system may be used to separate a mixture of proteins bearing multiple different purification tags simultaneously. In some embodiments the ligand for a common protein tag is an aptaswitch, which fluoresces upon binding of the target, so that the amount of a purified protein bound to the condensate layer can be easily quantified during purification.

In some embodiments, multiple layers of nucleic acid condensates are used to separate multiple high value proteins from a single mixture. For example, a three-layer system where each layer carries a distinct aptamer can be used to separate three different blood factors, where three different blood factors are chosen from the list: Factor II, Factor VII, Factor IX, Factor X, Factor XII and Factor H.

In some embodiments, an adaptor molecule capable of binding the Fc domain of an antibody is used to load an antibody ligand into the condensate layer. In some embodiments this adaptor is an Fc-binding protein, in others it is an Fe-binding aptamer. In some embodiments a multilayer system is used, and each layer has an antibody raised against the antibodies of a particular animal. For example, in a three-layer system, the top layer might have anti-goat IgG antibodies, the middle layer might have anti-rabbit IgG antibodies, and the bottom layer might have anti-mouse IgG antibodies. These embodiments enable any target for which an antibody can be raised to be separated in a single or multiple-layer condensate system, where the target can be addressed to a particular layer in a modular fashion. This embodiment enables a customizable purification system to be created, where a user simply adds appropriate antibodies to an existing set of layers to purify any desired set of three protein targets (for which antibodies could be raised or commercially purchased).

In some embodiments, the antigen for an antibody or cell-surface receptor is used as a ligand in the nucleic acid condensate. In such an embodiment the antigen ligands can be used for a number of applications: (1) purification of an antiviral antibody for use as a therapeutic, e.g., SARS-CoV-2 spike protein could be used as a target, (2) detection of antibodies in patient blood, either against a pathogenic agent or autoantibodies, (3) purification/selection of cells engineered to bind the antigen, e.g. chimeric antigen receptors (CAR) T cells.

In some embodiments, a nucleic acid condensate with aptamers specific to a particular bacteria may be used to separate bacteria from blood for diagnosis of sepsis. Different condensates with aptamers to different bacterial strains may be used in a multi-layer system.

In some embodiments methods are described of separating at least three distinct targets from a mixture of targets comprising synthesizing at least three sets of condensate monomers configured to forming at least three condensates that layer distinctly from each other, wherein at least one condensate monomer in each set has a ligand for one of the targets m each set of condensate monomers for a layer contains at least one type of monomer.

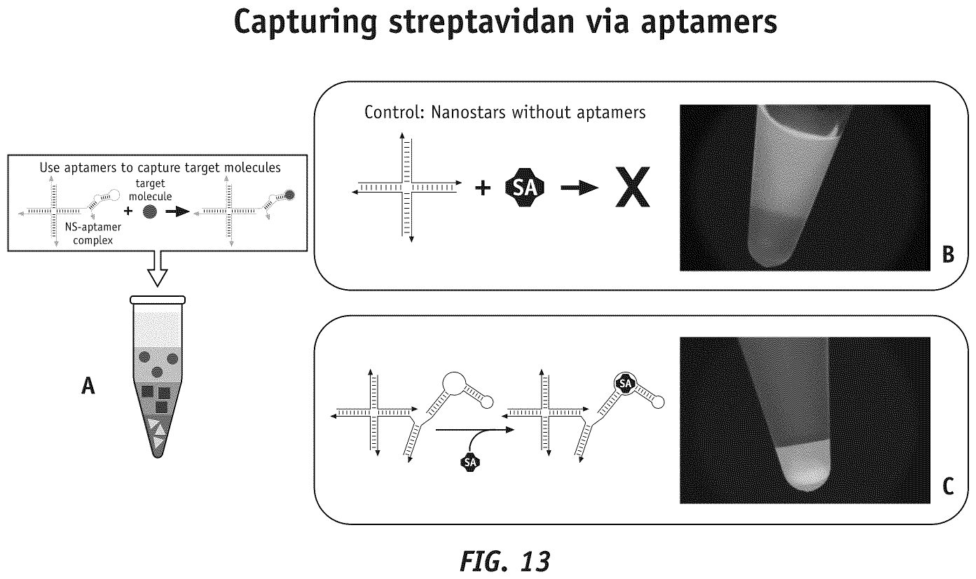

The method further comprises contacting a mixture of targets to the synthesized monomers and incubating until the ligands on the monomers bind the targets, performing a step that induces condensation and layering and performing at least one removal of the undesired supernatant. For example, in the case of streptavidin capture and release (FIG. 13 through FIG. 17), the condensate is operated near room temperature. The strands to form the condensate are mixed with each other, and with streptavidin, before any salt was added. Upon addition of salt (e.g. 0.5 M), the condensate induced to form and was incubated for 1 hour before centrifugation was performed, again at room temperature. Release was similarly performed, except it was performed at or below room temperature over the course of a week.

IN some embodiments, the method can also comprises performing optional washes of the condensates by introducing clean buffer, mixing the buffer with the condensates and inducing layering again and performing an optional step to separate the layers of the condensates by pipetting the at least three layers into at least three separate tubes and release of the targets from the condensate.

In some embodiments, in the method to separate purify and/or detect a biomolecular target in accordance of the disclosure, condensation and layering is induced by the addition of salt.

In some embodiments, in the method to separate purify and/or detect a biomolecular target in accordance of the disclosure, condensation and layering is induced by centrifugation.

In some embodiments, in the method to separate purify and/or detect a biomolecular target in accordance of the disclosure, each condensate for each layer is comprised of a binary mixture of two types of monomers, one type of which is mixed with the targets in the incubation step and the other type of monomer which is added (for each condensate) in the condensation and layering step.

In some embodiments, in the method to separate purify and/or detect a biomolecular target in accordance of the disclosure, condensation and layering is induced by thermal incubation or annealing.

In some embodiments, in the method to separate purify and/or detect a biomolecular target in accordance of the disclosure, the targets are nucleic acids, release of the biomolecular target is performed by serially adding nucleic acid strands that displace the targets one at a time, with each displacement being followed by collection of the supernatant

In some embodiments, in the method to separate purify and/or detect a biomolecular target in accordance of the disclosure, the ligands are aptamers, release of the biomolecular target is performed by serially adding nucleic acid strands that displace the targets one at a time via a kleptamer reaction, with each kleptamer displacement being followed by collection of the supernatant.

In some embodiments, in the method to separate purify and/or detect a biomolecular target in accordance of the disclosure, comprising performing the options washing of the condensate the targets are nucleic acids, and parallel strand displacement reactions are performed on each of the at least three separate tubes and the supernatant is collected.

In some embodiments, in the method to separate purify and/or detect a biomolecular target in accordance of the disclosure, the method comprises performing the options washing, the ligands are aptamers, and parallel kleptamer reactions are performed on each of the at least three separate tubes and the supernatant is collected.

In some embodiments a method can be performed separating at least three distinct targets from a mixture of targets comprising synthesizing at least three sets of condensate monomers capable of forming at least three condensates that layer distinctly from each other, wherein at least one condensate monomer in each set has a ligand for one of the targets. Each set of condensate monomers for a layer contains at least one type of monomer.

The method further comprises mixing the condensate monomers and performing a step that induces their condensation and adding a complex mixture of targets to the monomers, stirring this mixture into the preformed condensates and incubation until the ligands on the monomers bind the targets. The method also comprises performing a step that induces layering of the condensates as described herein, and performing at least one removal of the undesired supernatant and performing release of the targets from the condensate.

In some embodiments the method can also comprise performing optional washes of the condensates by introducing clean buffer, mixing it with the condensates and inducing layering again, performing an optional step to separate the layers of the condensates by pipetting the at least three layers into at least three separate tubes before performing release of the targets from the condensate.

In some embodiments, a method is described of detecting at least three distinct targets, which comprises synthesizing at least three sets of condensate monomers capable of forming at least three condensates that layer distinctly from each other, wherein at least one condensate monomer in each set has a ligand for one of the targets. Each ligand is an aptaswitch which fluoresces brightly upon binding of the target, each set of condensate monomers for a layer contains at least one type of monomer, each aptaswitch having a distinct fluorophore with a distinct emission spectra.

The method further comprises adding a complex mixture of targets to the monomers and incubating until the ligands on the monomers bind the targets, performing a step that induces condensation and layering and detecting the target molecule bound to the ligand of the functionalized condensate nucleic acid condensate.

In some embodiments, the detecting can be performed within the functionalized nucleic acid condensate (e.g. by imaging, fluorescence microscopy and/or single-molecule tracking). For example n some embodiment the detection can be performed in situ within the functionalized nucleic acid condensate imaging the one or more layered condensate and measuring the fluorescence from each layer as an indication of how much target of each type was present and therefore for quantitative detection of one or more targets within the one or more layered condensates.

In some embodiments, the detecting can be performed following separating the target molecule from the functionalized nucleic acid condensates.

A skilled person will be able to select both in situ analysis of molecular behavior within condensates and traditional separation-based detection techniques depending on the target molecule, and the experimental design.

In embodiments where a functionalized layered nucleic acid condensate structure of the disclosure is used to performed separations with a single condensate layer, it is a preferred embodiment/practice to remove the diffuse layer after layering. In those set of embodiments case, the diffuse layer serves as a repository for molecules that are unwanted. While some unwanted molecules are small enough or appropriately charged to have a significant concentration within the condensate layer, many of a sufficient size will be excluded from the condensate layer and be primarily concentrated in the diffuse layer. Accordingly in those embodiments, removal of the diffuse layer. (Or to move the condensate layer itself to a fresh tube before release of the target from the condensate layer is preferred.

In methods and systems herein described a separation of a mixture of in the sense of the disclosure can be performed for analytical purposes, and therefore be directed to qualitatively or quantitatively detect at least one component of the source mixture.

The terms “detect” or “detection” as used herein indicates the determination of the existence, presence or fact of a target item in a limited portion of space, such as a sample, a reaction mixture, a molecular complex and a substrate as well as one or more biological features thereof. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “qualitative” when it refers, relates to, or involves identification of a quality or kind of the target or signal in terms of relative abundance to another target or signal, which is not quantified, such as presence or absence. A detection is “quantitative” when it refers, relates to, or involves the measurement of quantity or amount of the target or signal (also referred as quantitation), which includes but is not limited to any analysis designed to determine the amounts or proportions of the target or signal.

When an affinity-binder based approach to analysis is used (as is used here in the case of layered condensates), the figures of merit for the qualitative or quantitative detection of biomolecular targets, such as sensitivity, accuracy, specificity, and limit of detection are in large part determined by the quality of the binders (the aptamers, antibodies, haptens, etc.) that are used. Thus the particular values of the figures of merit that can be achieved with the system will be similar or proportional to those achievable using the same binder in other settings. A first antibody to COVID spike protein that performs 10× better than a second antibody in the context of a lateral flow test for COVID will likely perform 10× better in the context of the layered condensate systems described here. Subsequent uses that can benefit from high accuracy, high sensitivity, high specificity, and low limit of detection comprise for example diagnosis or determination of occurrence of biological event following detection of biomolecular targets comprising biomarker. Examples include measurement of troponin which peaks in concentration 3-12 hours after a heart attack, or prostate specific antigen whose elevated level in blood is an indicator of prostate cancer.

In some embodiments, detection in layered condensate systems is by fluorescence. In some embodiments fluorescence is generated upon target binding via the actuation of a fluorescent aptamer switch whose conformational change upon binding entails increased fluorescence. In some embodiments the increase in fluorescence is mediated by the dequenching of a fluorophore, the fluorophore being part of a quencher fluorophore pair, and whose distance increases when the target binds the aptaswitch. In such embodiments, fluorescence signal appears in a signal step, when the target contacts the appropriate condensate monomers. In other embodiments fluorescence signal is generated in an additional second step. Such embodiments are preferred when aptaswitches cannot be obtained. Instead a set of fluorescent secondary binders (e.g. antibodies, at least one for each target) are contacted to the layered condensate (mixed with the condensate). The secondary binders bind the targets at a second epitope and label the condensate-bound targets fluorescently. The condensate is washed to remove the secondary binders and then relayered to provide sensitive fluorescent detection of the targets with low background.

Methods are herein described to manufacture a set of functionalized nucleic acid condensate monomers of the present disclosure configured to specifically bind a biomolecular target and to form a functionalized nucleic acid condensate having a distinct density.

The method comprises providing for each nucleic acid condensate monomer, n nucleic acid strands with n being an integer from 1 to 12 and configured to form a nucleic acid condensate monomer in the sense of the disclosure

The methods further comprises attaching the ligand to the targeting domain to provide the functionalized set of nucleic acid condensate monomers of the disclosure and optionally inducing condensation of the functionalized set of nucleic acid condensate monomer to form a functionalized nucleic acid condensate, detecting a density of the functionalized nucleic acid condensate to detect the distinct density of the functionalized nucleic acid condensate.

Functionalized nucleic acid monomers herein described and in particular nanostar structure can be designed and manufactured based on techniques described herein and/or identifiable by the skilled person upon reading of the present disclosure. In particular the configuration of the segments of the constructs can be identified and designed based on calculation of the thermodynamic stability of the various conformation of the segments and constructs as a whole. For example, thermodynamic stability of polynucleotide conformation dependents on several factors identifiable by a skilled person, including its i) chemical composition (for example, DNA:RNA duplex is less than RNA:RNA duplex); ii) base composition (for example, G/C base paring is more stable than A/T base paring, which is approximately as stable as G/T, G/U wobble base pairing, and the formation of a stable RNA hairpin requires at least 3 G/C base pairs or at least 5 A/U, G/U base pairs); iii) nearest neighbors such as presence of mismatches, open ends, and junctions near a base-pair can substantially influence its energy contribution according to the second-nearest neighbor model (for example, the stacking of successive base-pairs is primarily responsible for the stability of DNA helices); iv) non-canonical base pairing (for example, RNA and DNA can form triple helix and quadraplex structures via Hoogsteen base-pairing, which is less stable base pairings than canonical base pairing); v) Geometry (e.g. polynucleotide sequences can only adopt secondary structures that are geometrically consistent or similar with the known tertiary structural characteristics of RNA and DNA helices); vi) Environmental factors, such as pH value, counter-ion concentration and temperature and additional factors identifiable by a skilled person.

Accordingly, designing the polynucleotide sequences comprised in the functionalized nucleic acid monomers herein described can be performed identifying the combination of length, sequence, complementarity and substitutions that is associated with a desired relative thermodynamic stability resulting in the configuration herein described in the environment wherein the condensate forms and when the enzyme assisted release is desired. Specific sequences of desired release polynucleotides can be identified by a skilled person based on environment (and in particular, specific cells and tissues) where delivery is desired. In some embodiments, polynucleotide sequences can be designed according to the corresponding physiological conditions, such as approximately, pH 7.3-7.4, about 150 millimolar potassium or sodium chloride or equivalent salt, and about 37° C.

For base pairing between unmodified DNA segments or between unmodified RNA segments, the base-pairing energies and the most stable secondary structure conformations can be estimated by computational methods known to and well established in the art. Several packages are available and published in documents also discussing in detail factors affecting the energy and stability of nucleic acid secondary structures. Exemplary publications describing the packages and factors comprise for i) NUPACK web server: [20]); ii) NUPACK analysis algorithms: [21]; [23]; iii) NUPACK design algorithms: [24] iv) mfold web server: [25] [26]; [27]v) UNAFold & mfold: [28]; [29] [30] [31]); [M. Zuker, Science 244, 48-52, (1989)]; vi) Free energies for RNA: (see e.g. D. H. Mathews et al., J. Mol. Biol. 288, 911-940, (1999)]; and A. E. Walter et al., Proc. Natl. Acad. Sci. USA 91, 9218-9222, (1994)]; vii) Methods and theory of RNA secondary structure prediction: (see e.g. D. H. Mathews et al., Secondary Structure Prediction. In Current Protocols in Nucleic Acid Chemistry S. Beaucage, D. E. Bergstrom, G. D. Glick, and R. A. Jones eds., John Wiley & Sons, New York, 11. 2. 1-11. 2. 10, (2007); D. H. Mathews et al., Predicting RNA Secondary Structure. In The RNA World, R. F. Gesteland, T. R. Cech and J. F. Atkins eds., 3rd edition, Cold Spring Harbor Laboratory Press, Cold Spring Harbor, New York, Chapter 22, (2006)]; D. H. Mathews et al. 3rd edition, John Wiley & Sons, New York, Chapter 7, (2005); D. H. Mathews et al., (2004); M. Zuker, Bull. Mathematical Biology 46, 591-621, (1984); M. Zuker et al., Nucleic Acids Res. 9, 133-148, (1981) and D. H Mathews et al Folding and Finding RNA Secondary Structure in Cold Spring Harb Perspect Biol. (2010)); viii) Exemplary mfold & UNAFold applications: (see e.g. J.-M. Rouillard et al., Nucleic Acids Res. 31 (12), 3057-3062 (2003); and J.-M. Rouillard, et al., Bioinformatics 18 (3), 486-487, (2002). In addition, since some polynucleotide structures typically fluctuate between an ensemble of secondary structure conformations, the composition of the relevant ensemble can be determined using computational methods known in the art (see for example, see Sfold web server for statistical folding and rational design of nucleic acids. Nucleic Acids Res. 32 Web Server issue, W135-W141, (2004), and Ye Ding et al., RNA 11, 1157-1166. 2005, herein incorporated by reference in its entirety).

Accordingly, in several embodiments, design of a polynucleotide sequence of the targeting and interaction domains of the functionalized nucleic acid monomers herein described, can be performed for sequences or portions of sequences consisting of unmodified DNA and/or RNA base pairs, by computational methods and/or software packages to calculate the free energy of the sequence and the secondary structure conformation. In embodiments, wherein polynucleotide sequences comprise derivatives of nucleotides, such as chemically modified bases and analogues, and/or chimeric polynucleotide sequences composed of a mixture of deoxyribonucleotides and ribonucleotides, design can be performed by computationally designing unmodified RNA structures with the desired secondary structure conformations and thermodynamic stability, and then introducing one or more chemical modifications to achieve the desired thermodynamic stability.

Attachment of the functionalized nucleic acid monomers to other nucleic acid binding components such as polypeptides (see e.g. Tan, R. & Frankel, A. D. in Proceedings of the National Academy of Sciences 92, 5282-5286 (1995) proteins (see e.g. Castello, A., in. Cell 149, 1393-1406, (2012) and Dreyfuss, G., et al in Nat Rev Mol Cell Biol 3, 195-205 (2002)), cationic polymers, or RNA structures such as ribosome (e.g. Yusupov, M. M., et al in. Science 292, 883-896, (2001) and Kahan, M., et al in Physica D: Nonlinear Phenomena 237, 1165-1172 (2008)), and tRNA (Scherer, L. J., in Nucleic Acids Research 35, 2620-2628, (2007)) can also increase targeting of saRNA sample RNA transcripts in the cell.

The functionalized nucleic acid monomers designed according the present disclosure can be synthesized using standard methods for oligonucleotide synthesis well establish in the art, for example, see Piet Herdewijn (2005), herein incorporated by reference in its entirety.

The synthesized oligonucleotide can be allowed to form its secondary structure under a desirable physiological condition, (e.g. 1× phosphate buffered saline at pH 7.5 with 1 mmolar concentration MgCl2 at 37° C.). The formed secondary structure can be tested using standard methods known in the art such as chemical mapping or NMR (see e.g. Kertesz, M., et al in. Nature 467, 103-107, (2010), (see e.g. Mathews, D. H., et al in. Cold Spring Harbor Perspectives in Biology 2, (2010), and (see e.g. Watts, J. M., et al in. Nature 460, 711-716, (2009)). For example, see (R see e.g. ef Stephen Neidle, Neidle, S. Principles of nucleic acid structure. (Academic Press, 2010) herein incorporated by reference in its entirety. The designed construct can be further modified, according to the test result, by introducing or removing chemical modifications, mismatches, wobble pairings, as necessary, until the desired structure is obtained. Reference is made in this connection to the exemplary procedure provided in the Examples section

s In some embodiments of a functionalized nucleic acid monomer can be performed by covalently attaching the ligand to a targeting domain the monomer through a covalent linkage formed by typical bioconjugate methods as described in Hermanson, G. T. 2008 [32]. For example, the linkage can comprise a covalent bond or bonds such as a carbon-oxygen bond as found for ester, ether, anhydride and phosphodiester linkages; a carbon-nitrogen bond as found for amides, ureas, diimides and triazole linkages; or a carbon-sulfur bond as found for thiol, sulfide, disulfide and thio-succinimide linkages. Alternatively, the linkage can comprise a coordination bond between a nucleotide base and a metal complex.

In some embodiments manufacturing a functionalized nucleic acid monomer can be performed by covalently attaching the ligand the targeting domain of the nucleic acid strand before being combined with other nucleic acid strands to form mixtures of the current invention. Alternatively, one or more nucleic acid strands in a mixture may be modified by covalent attachment to the ligand using a suitable method such as orthogonal conjugation or a ligase.

In some embodiments a method of manufacturing condensate monomer can be performed to manufacture a functionalized set of nucleic acid condensate monomers of the present disclosure having a set distinctive density.

The method comprises manufacturing a set of functionalized nucleic acid condensate monomers according to a method of the disclosure inducing condensation set of functionalized nucleic acid condensate monomers to form a functionalized nucleic acid condensate, detecting a density of the at least one functionalized nucleic acid condensate and comparing the detected density with the set distinctive density.

The method optionally further comprises modifying the structure of at least one monomer of the set of functionalized nucleic acid condensate monomers when the detected density is different from the distinctive density, and performing the manufacturing the inducing, and the detecting to determine a modified detected density followed by comparing the modified detected density with the set density.

The method further optionally comprises repeating the modifying, the manufacturing, the inducing, the detecting and the comparing until the modified detected density correspond to the set density

The system comprises a system to manufacture a set of functionalized nucleic acid condensate monomers of the disclosure herein described together with condensation agents devices to detect the polynucleotide condensate density and agents to modify the functionalized nucleic acid condensate monomers

As disclosed herein, the functionalized nucleic acid monomers components herein described as well as release agents and related reagents and/or devise to perform the methods of the disclosure can be provided as a part of systems for performing anyone of the methods herein described. The word system as used herein indicates a combination of components configured to perform one or more steps of the methods herein described.

The systems can be provided in the form of kits of parts. In a kit of parts, the functionalized nucleic acid monomers and related components and other reagents to perform preparation, separation, purification and/or detection can be comprised in the kit independently. The functionalized nucleic acid monomers can be included in one or more compositions, and each construct or component can be in a composition together with a suitable vehicle.

The term “vehicle” as used herein indicates any of various media acting usually as solvents, carriers, binders or diluents for functionalized nucleic acid monomers and related components that are comprised in the composition as an active ingredient. In particular, the composition including the functionalized nucleic acid monomers and related components can be used in one of the methods or systems herein described.

In some embodiments, systems of the disclosure can comprise nucleic acids, ligands, related functionalized or unfunctionalized nucleic acid condensate monomers, agents for attaching ligands to monomer targeting domains of nucleic acid condensate monomers. devices for highlighting layers with bound targets and/or additional condensation agents,

In some embodiments, systems in the sense of the disclosure can comprise functionalized or unfunctionalized nucleic acid monomers herein described condensation agents, and for each of the at least one targeting domains and the ligand presented on the functionalized or unfunctionalized nucleic acid monomers, an optional agent for attaching the ligand to the nucleic acid strand, and/or (3) for each of the at least one targeting domains of the functionalized or unfunctionalized nucleic acid monomers and the ligand it presents, an optional devices to indicate or measure the localization of biomolecular targets to the condensate monomer bearing the corresponding ligand for the biomolecular target” or similar.

In some embodiments “condensation agents” can comprise counterions/salts which can be used for example in embodiments where the biomolecular targets comprises proteins that are sensitive to thermal annealing.

In some embodiments, condensing agents and/or releasing agents can be present or omitted in the system when the system is directed to performed layering step in a single layer or multilayer macroscopic condensate through annealing or centrifugation, or both, or through the addition of a condensing reagent, as will be understood by a skilled person

Additional components can include labeled polynucleotides, labeled antibodies, labels, reference standards, and additional components identifiable by a skilled person upon reading of the present disclosure.

The terms “label” and “labeled molecule” as used herein refer to a molecule capable of detection, including but not limited to radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like. The term “fluorophore” refers to a substance or a portion thereof which is capable of exhibiting fluorescence in a detectable image. As a consequence, the wording “labeling signal” as used herein indicates the signal emitted from the label that allows detection of the label, including but not limited to radioactivity, fluorescence, chemoluminescence, production of a compound in outcome of an enzymatic reaction and the like.

In embodiments herein described, the components of the kit can be provided, with suitable instructions and other necessary reagents, in order to perform the methods here disclosed. The kit will normally contain the compositions in separate containers. Instructions, for example written or audio instructions, on paper or electronic support such as tapes, CD-ROMs, flash drives, or by indication of a Uniform Resource Locator (URL), which contains a pdf copy of the instructions for carrying out the assay, will usually be included in the kit. The kit can also contain, depending on the particular method used, other packaged reagents and materials (i.e. wash buffers and the like).

### EXAMPLES

The functionalized nucleic acid monomers herein disclosed are further illustrated in the following examples, which are provided by way of illustration and are not intended to be limiting.

### Example 1: DNA Nanostars Phase Separate to Form Liquid

An exemplary sample separation procedure using unfunctionalized DNA nanostars structure is illustrated herein from the reference Biffi et al 2013 [1]

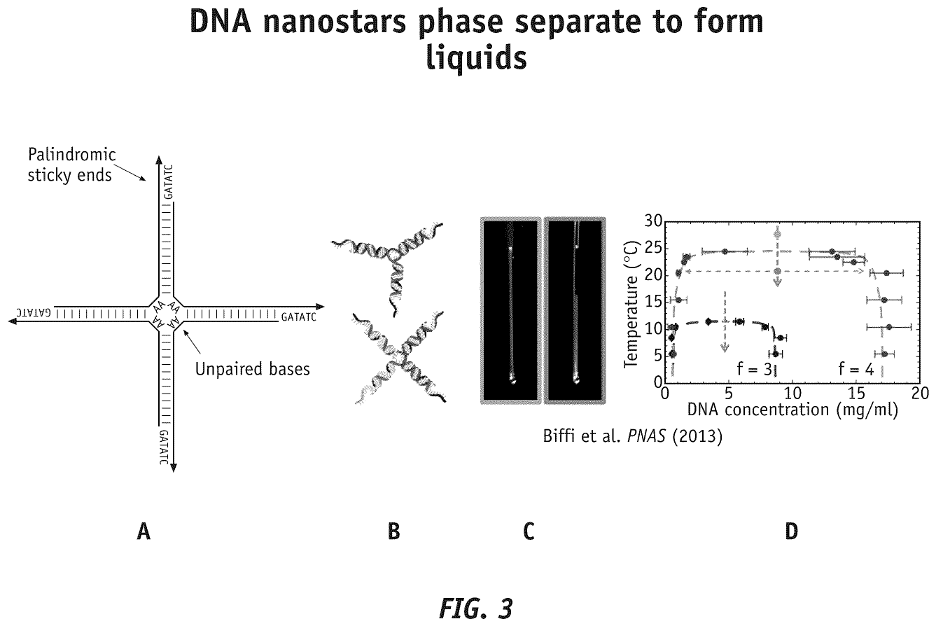

DNA nanostars were created with valence 3 and 4 where created and then allowed to condensate A schematic illustration of the nanostars used in Biffi et al 2013 [1] is reported in FIG. 3 which shows, a schematic representation of a four armed nanostars (FIG. 3 Panel A), a schematic illustration of the f=3 f=4 nanostars used formed by the self-assembly of three and four oligomers, (FIG. 3 Panel B).

In particular, FIG. 3 Panel A shows a schematic architecture for a 4-arm nanostar. Arms are shown as having only 13 nt arm length but the condensates in this figure actually have 20 nucleotide arm length. Two adenine (‘AA’) flexible linkers at the junctions between arms are shown. Single adenine (‘A’) flexible linkers between the terminus of the arms and the palindromic GATATC sticky ends are used but are not diagrammed. FIG. 3 Panel B shows schematics for 3-arm and 4-arm nanostars.

The related condensate was created by mixing equimolar quantities of 49-nucleotide-long oligomers. The preparation used 35 mM NaCl for f=3 structures and 25 mM NaCl for f=4 structures, with both systems reaching a final ionic strength of 50 mM NaCl. The oligomers formed structures with double-stranded arms of 20 base pairs each. Unpaired adenine bases were inserted between the arms to provide flexibility. [1]

The phase separation was carried out by first loading 7-8 uL of solution into glass microcapillary pipettes. Both ends of the pipettes were then flame sealed. The samples underwent heating at 90° C. for approximately 20 minutes, followed by centrifugation at 3,000×g for about 4 hours to speed up the phase separation process. [1]:

Solutions obtained with the above approach reportedly exhibited phase separation below specific temperatures, forming DNA-rich and DNA-poor phases. (see Fluorescent emission from a capillary tube of (FIG. 3, Panel C) Critical temperatures were determined to be: f=3 nanostars: Tc=11.6±0.1° C., f=4 nanostars: Tc=25.5±0.1° C. [1] (FIG. 3, Panel D)

In particular, FIG. 3 Panel C shows capillaries having condensates comprising 3-arm (at left) and 4-arm (at right) nanostar liquids. For the same input number of molecules the 3-arm condensate creates a lower density, higher volume phase, lower fluorescence intensity liquid phase that almost fills the capillary, while the 4-arm condensate is much denser, higher fluorescence liquid phase that occupies at most the bottom 3rd of the tube.

FIG. 3 Panel D shows experimental gas-liquid coexistence curves for the f=3 (3-arm) and f=4 (4-arm) condensates. Inside the inverted U-shaped curves the nanostars are liquid. Outside of these curves the nanostars make a dilute gas phase. The liquid for 3-arms has a 200 uM concentration (8.5 mg/ml on the graph, which has been calculated taking the molecular weight of the nanostar into account), and the liquid for the 4-arm with the same sticky ends is a denser 290 uM (17 mg/ml on the graph, which has been calculated taking the molecular weight of the nanostar into account, having an extra arm) concentration. The area of the gas/liquid phase coexistence region for 4-arm nanostars (0-24 degrees C., 0.5 mg/ml to 17 mg/ml) is much larger than that for the 3-arm nanostars (0-10 degrees C., 8.8 mg/ml), encompassing both higher temperatures and higher initial DNA concentration.

Analysis of phase characteristics revealed several key findings about network structures. The dense phase concentration matched theoretical predictions for fully bonded networks. The f=3 structures exhibited a smaller coexistence region than f=4 structures, and the concentration range for separation decreased when comparing f=4 to f=3 structures. [1]

Examination of dynamic behavior showed that solutions near the critical point underwent two-step relaxation processes. The slow relaxation component exhibited Arrhenius-activated behavior instead of critical slowing down. Additionally, the bond lifetime between DNA structures was found to be a determining factor in the system's dynamics [1]

This research of Biff et al 2013 provide proof of principle that DNA nanostars serve as an excellent model system for studying limited valence particles, offering precise control over binding selectivity, interaction strength, and valence through DNA sequence design. [1]

### Example 2: Condensates Act as a Compartment that Host Molecules

Experiments have been performed, discussed in Biffi et 2013 [1], Alberti et al 2017 [2] Jeon et al [4] and Sato et al 2019 [3] which have provided proof of principle of how condensates can act as a compartment that host molecule.

In Biffi et al 2013 [1] exemplary DNA nanostars were formed through self-assembly of 49-nucleotide oligomers, creating structures with either 3 or 4 arms (f=3 or f=4). Each arm consists of 20 base-paired nucleotides, with unpaired A bases inserted between arms to provide flexibility. Arm tips terminate with one sticky overhang each. The structures were shown to maintain open conformations due to electrostatic repulsion between phosphate groups, with f=4 forming tetrahedral shapes and f=3 forming flat structures.

Further experiments characterization and considerations have been reported discussed in Alberti et al 2017 [2] Jeon et al [4] and Sato et al 2019 [3].

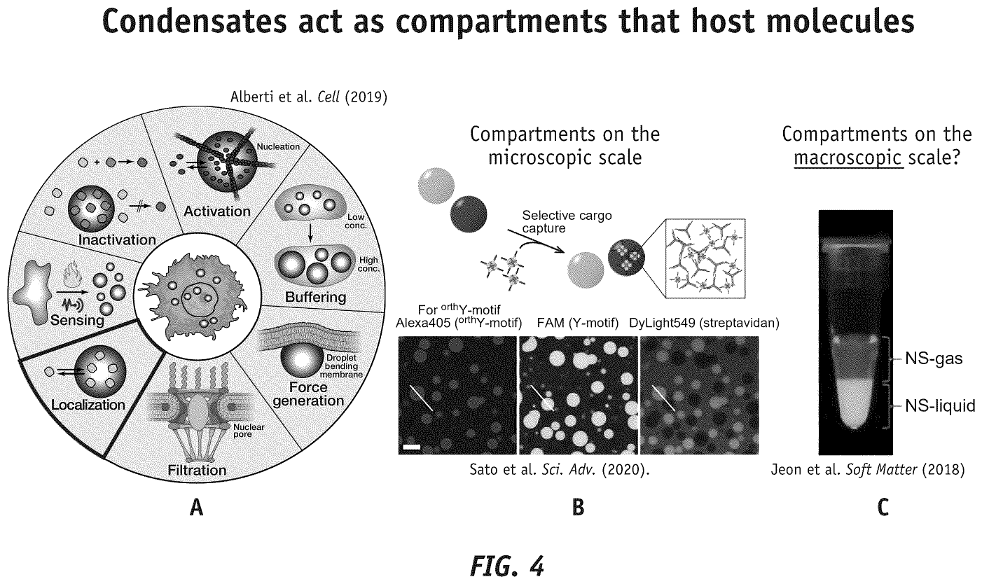

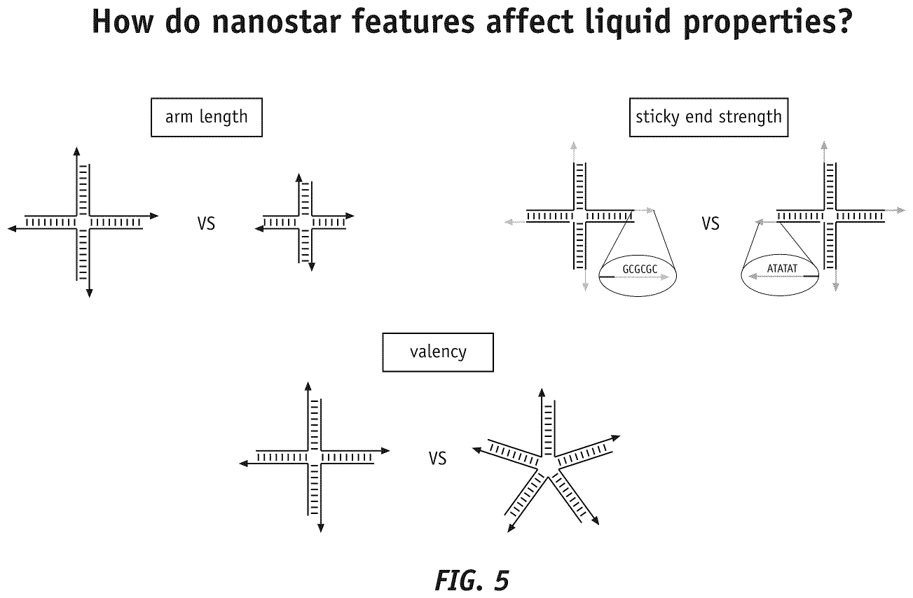

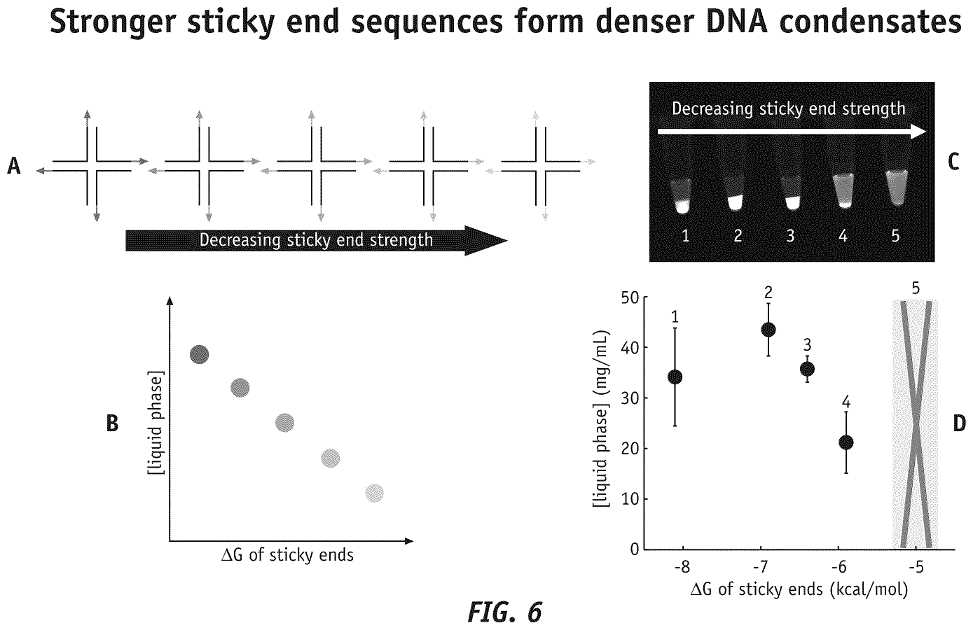

Accordingly, a skilled person understands DNA nanostars can form membraneless microcompartments through several key mechanisms that work together to create functional cellular structures. A schematic representation of the biomolecular condensation and its features is reported in FIG. 4, showing an illustration of the related repertoire from FIG. 4 of Alberti et al 2017 [2] (FIG. 4 Panel A), an illustration of the compartments on a microscopic scale formed by the microscopic images of DNA droplets from FIG. 5C and the schematics of FIG. 6B. of Sato et al 2019 [3] (FIG. 4 Panel b), and an illustration of the compartments on a macroscopic scale formed by a Bulk phase separation of 100 μL of NS solution. NSs are visualized by adding YOYO-1 at 1:100 dye: NS molar ratio from FIG. 1A of Jeon et al [4] (FIG. 4 Panel C).

In particular, FIG. 4A shows the broad range of functions that are served by condensates in biology. Of particular interest here is the blue highlighted wedge in which molecules (yellow dots) are localized to a biological condensate (green); biomolecular condensates act as compartments that attract and host guest molecules.

FIG. 4 Panel B top shows a schematic figure from Sato et al, in which streptavidin molecules are localized to the blue condensate but not the green condensate. In FIG. 4 Panel B bottom, micrographs (30 micron scale bar) show the localization of streptavidin molecules to one of two microscopic condensates but not the other.

Particularly relevant to the use of artificial condensates to compartmentalize targets is FIG. 4, Panel B, where streptavidin is compartmentalized in a DNA condensate. FIG. 4, Panel B top shows a schematic figure from Sato et al, [3] in which streptavidin molecules are localized to the blue condensate but not the green condensate. In 4B bottom, micrographs (30 micron scale bar) show the localization of streptavidin molecules to one of two microscopic condensates but not the other.

FIG. 4 Panel C relates to use DNA liquid condensates to separate bulk quantities of a target molecule. To do so, macroscopic quantities of liquid are first required. FIG. 4 Panel C shows that bulk DNA condensates (without target molecules) can be experimentally formed. Macroscopic volumes of condensates can be synthesized and separated by liquid-liquid phase separation within a microcentrifuge tube (here 200 uL capacity). The nanostar liquid appears as a opaque green layer at the bottom (with nanostar condensate monomers in the 100-500 micromolar concentration range), labelled NS-liquid underneath a diffuse NS-gas layer (transparent, but here appearing a lighter green as there is a much lower 1-2 micromolar concentration of nanostar condensate monomers).

The approach illustrated in FIG. 4 relates to the use of DNA condensates to compartmentalize molecules However according to the approach illustrated in FIG. 4, naked streptavidin molecules are not localized to the microscopic compartment. Instead the streptavidin (red Xes) have been covered in four DNA arms (blue), where each DNA arm is designed to bind to nanostars of the blue DNA condensate. In this scheme, the streptavidin have been functionally turned into DNA nanostars, with the tetravalent streptavidin serving as the junction for the arms. There is no mechanism for release or removal of the streptavidin, alone, from this system.

In contrast, condensate monomers of the present disclosure, use of ligands on the nanostars that comprise the condensate; which allows a controlled compartmentalization of the target as we well as the selective removal of the target, (e.g. by strand displacement). Additionally because target for compartmentalization do not in general have the multivalent structure of streptavidin, which enables it to be turned into a condensate-soluble 4-armed nanostar, the condensate monomers of the disclosure allow compartmentalization and release an increased number of targets.t

At their core, nucleic acid condensate which are functionalized in accordance with the present disclosure, are compartments which arise through liquid-liquid phase separation (LLPS), generating dense phases that coexist with dilute phases while maintaining distinct boundaries through surface tension. The structural organization of these compartments is remarkably sophisticated. They form without requiring membranes, instead relying on selective properties that control which proteins and RNAs can enter or be excluded. This molecular sorting depends on sequence features, surface tension between phases, and multivalent interactions between components. Within these compartments, the internal organization shows remarkable complexity. They can develop into multilayered structures with distinct subcompartments, each having different material properties ranging from highly fluid to more viscous or gel-like states The components within these compartments remain dynamic, freely diffusing within the dense phase and exchanging with the dilute phase outside.

These structural properties enable several key functional capabilities. The compartments can concentrate specific molecules to accelerate biochemical reactions, create distinct chemical environments for specific cellular processes, and buffer protein concentrations through controlled partitioning They also act as mechanical and chemical filters with specific pore sizes and can generate mechanical forces that help shape cellular structures. The formation and properties of these compartments are precisely regulated through multiple mechanisms including changes in protein/RNA concentration, post-translational modifications, environmental conditions like pH and temperature, and specific molecular interactions between components.

This sophisticated control system allows cells to dynamically adjust compartment properties to meet changing cellular needs. This system has proven valuable for understanding the fundamental principles governing biomolecular condensates and their potential applications in synthetic biology and materials science.

### Example 3: Engineering Features and Configurations of Nucleic Acid Nanostars to Affect the Nanostar's Liquid Properties

Nanostar properties can be controlled by engineering the arm length, sticky end strength and valency of the arms of the nanostar as will be understood by a skilled person.

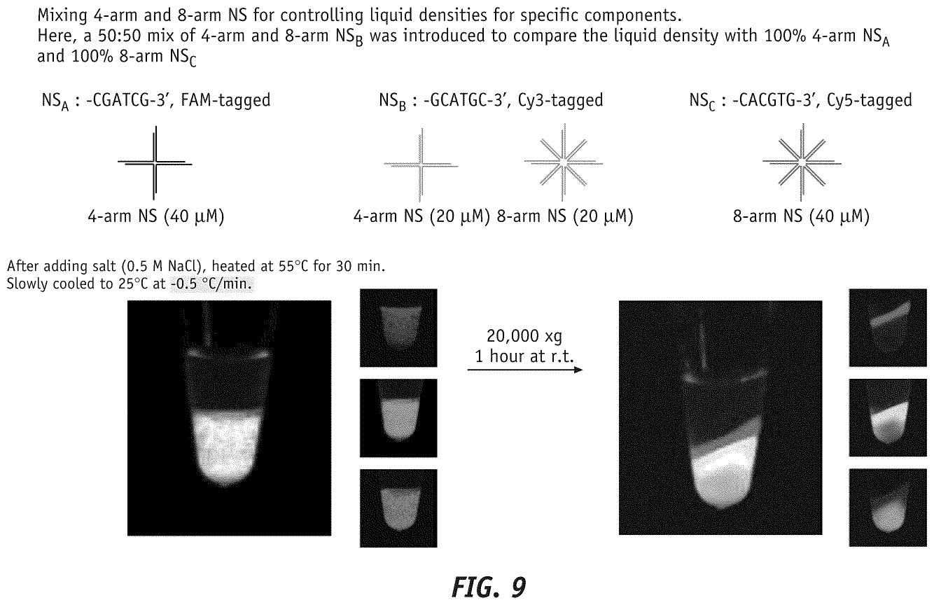

As is shown in schematic form in FIG. 5, several different structural features of nanostars affect liquid properties like density and melting temperature. Smaller nanostars (having shorter arm length) yield denser condensates, of higher melting temperature. Stronger sticky ends, having more GC on average, yield denser condensates of higher melting temperature. Nanostars having higher valence, that is, more active arms with sticky ends, are denser and have a higher melting temperature. Other properties not shown have similar effects. For example, higher arm number, and average arm number, create denser liquid condensates. For example, higher arm number, and average arm number, create denser liquid condensates. Note that nanostar valence does not equal arm number, because some arms may not carry interaction domains (not pictured here, but pictured in FIG. 9).

Results of experiments showing that stronger sticky ends form denser DNA condensates, are reported in FIG. 6. In particular, FIG. 6 shows that fundamental studies of a varying particular structural feature (here sticky end strength) with other features of the nanostar being held constant, can reveal fundamental trends in density which can then be used for layering. Similar studies have been performed for arm length and valence, which have allowed us to understand the sign of those trends, so that one skilled in the art can then use those trends to rationally engineer layered structures where a single variable is changed.

In particular the schematics of FIG. 6 Panel A, show an approach based on decreasing sticky end strength for 5 stars. The liquid phase concentration should decrease with decreasing sticky end strength as illustrated by the cartoon diagram of FIG. 6 Panel B of the liquid phase concentration (e.g. in mg/ml) vs strength of the sticky end (free energy, ΔG, e.g. kcal/mol, and the related experimental analog shown in FIG. 6 Panel D as will be understood by a skilled person.

Experimental data were retrieved for condensates having 5 different sticky end strengths ands reported in the illustration of FIG. 6 Panel C. In particular, 4 arm nanostars with an arm length of 20 were used. Each strand was 124.8 uM, buffer is 1× TE, Ph 7.5, 0.5 M NaCl, 1% YOYO-1 dye. 1 hour at 60° C., then ramped from 60 to 20 at 0.1° C./min. Spinning 1 hr at 20° C. at 40,000×g with 45-degree angled rotor. At room temperature dilute phase was pipetted out at room temperature and then put in a glove box at 50° C. Within glove box the dense phase was diluted 1:100 and then the absorbance was measured in a UV spectrophotometer and measured against a 5 mM NaCl buffer blank (because of the 1:100 dilution). The density was derived using known extinction coefficients. The strongest sticky end formed a gel and so the trend holds for nanostars labeled 2, 3, and 4.

The illustration of FIG. 6 Panel C and Panel D show how experiment agrees with and differs from the theoretical trend. The experimental trend clearly holds for nanostars 2, 3 and 4, with liquid phase concentration/density decreasing as sticky end strength decreases. However, no concentration was measured for nanostar 5 as it didn't condense into a liquid phase, or the amount of condensate created was so small it could not be measured (this is consistent with the trend). Nanostar 1 creates a condensed phase that it is off trend, and has a density that is lower than nanostar 2. Note in FIG. 6 Panel C the condensate for 1 is less uniform than 2. The condensate for nanostar 1 is a gel phase (because the sticky end is too strong) having different properties than the liquid phases for nanostars 2-4. If the data for FIG. 6 Panel C and Panel D were taken at 20° C. If the data for FIG. 6 Panel C and Panel D were taken at higher temperature, then nanostar 1 would form a liquid phase rather than a gel phase (however nanostar 4 would not have condensed). If they were taken at a lower temperature then nanostar 5 would have formed a measurable amount of liquid phase, but nanostar 2 would form a gel phase.

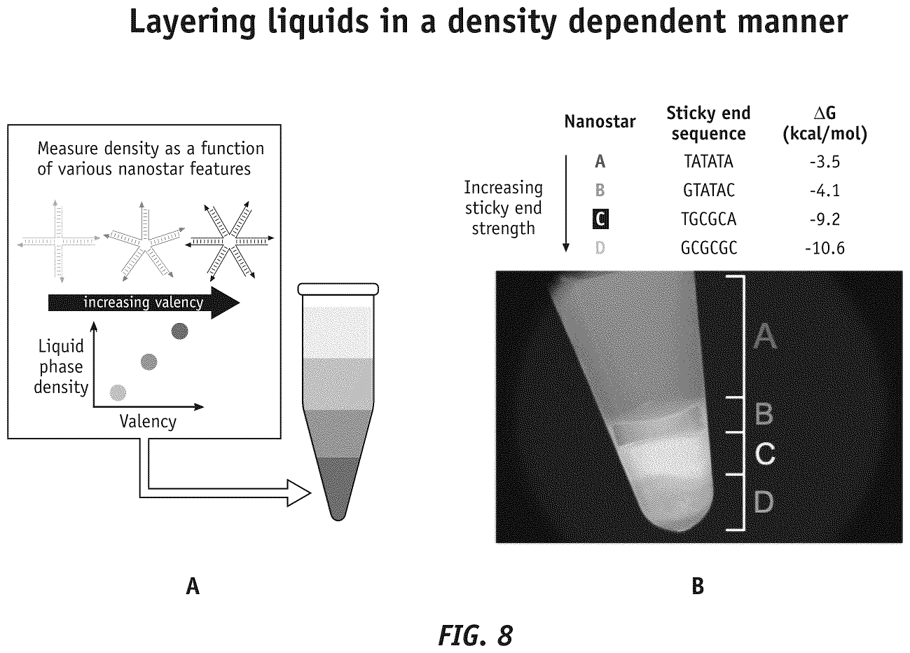

The exemplary results shown in FIG. 6, thus provide a showing that sticky end can be used to control density of a condensate formed by nucleic acid nanostar. Other features of a nanostar can be used to control condensate density as illustrated in FIG. 8 Panel A.

In particular FIG. 8 Panel A is shows that liquid phase density (e.g. in mg/mL) increases with the valency of the nanostars, which in this example also corresponds to the number of arms. In particular, FIG. 8 Panel A shows, in diagrammatic form, how a trend in density based on a single variable (valency) can be used to layer nanostars in a condensate. Because number of arms and valency correspond, the thermodynamics of these three nanostars vary systematically if the sticky ends all had the same ΔG per sticky end (the three-arm star would have 3 ΔG, the four arm 4 ΔG, and the five arm 5 ΔG); a greater interaction strength causes nanostars to bind more tightly and take up less space. Also, the number of arms is increasing so the molecular weight of each nanostar is increasing. In this case the density of the condensate would increase for two reasons (valencies effect on overall binding energy and the number of arms effect on molecular weight) but it would be impossible to disentangle the effects of number of sticky ends (valency) and number of arms. However, as shown in FIGS. 7 and 9, arm number or average arm number alone and not valency, is sufficient to drive layering (that is, it is not necessary to change valency and arm number at the same time for robust layering).

From the illustration of FIG. 8, a skilled person will understand that a plurality of condensates can be engineered operating on the structural features of a nanostar which can also be used for layering those condensates.

In this respect, FIG. 8 Panel B demonstrates that condensates can be layered on the principle of varying the strength of the sticky ends (the interaction domains), while leaving other structural features roughly constant. 4-arm, 24 nucleotide arm length condensate monomers were designed for four layers having ΔG of −3.5 (red, A), −4.1 (green, B), −9.2 (yellow, C) and −10.6 (orange, D) kcal/mol. Three of the four nanostars condensed, while the nanostar with the weakest sticky end strength (A, red) remained dispersed in the gas phase. This experiment demonstrates that ΔΔG as small as 1.4 kcal/mol (between layers C and D) can be used to provide robust layering. Each nanostar was at 41.6 uM, in 1× TE, pH 7.5, 0.5M NaCl. Thermal conditions were 1 hour hold at 60, followed by a ramp from 60° C. to 20° C. at 0.1 C/min. Centrifugation conditions were 40,000×g, 15 min spin in a 45-degree rotor at 20° C. followed by a second 3 min spin in a 90-degree rotor, 11,000×g at 20° C. (to flatten the layers relative to the tube axis). All tubes were 0.2 mL Corning PCR tubes. Using these small, thin-walled PCR tubes is important so that good fluorescence pictures can be taken using a fluorescent lamp at 302 nm.

Additional approaches for engineering and layering condensates in a density dependent manner are illustrated in Example 4.

### Example 4: Layering Liquids in a Density Dependent Manner, Based in Three Independent Structural Features

Nucleic Acid condensate engineered to have set density can be used to provide a layered structure which can be used for mixture separation. A particular structural feature is selected and varied holding others constant.

A first example of this approach is illustrated in FIG. 7 which exemplifies an embodiment where the density varies with the number of arms on a nanostar.

FIG. 7A provides a schematic of the nanostars used in the experiment. Arm lengths were all 20 nucleotides. Here only the four-arm nanostar NS-A was labelled fluorescently, so the 6-armed nanostar NS-B formed an unlabeled “dark” layer”. Sticky ends (5′GAGCTC-A and 5′ CGATCG-A) had similar free energies; both had single A flexible linkers. Note also that the 6-armed nanostars in this experiment each have 2 blunt arms and 4 active sticky-end bearing arms. Blunt-ends were introduced into these 6-armed stars to enable us to keep the thermodynamics of the 6-armed nanostars similar to those of the 4-armed stars, and isolate arm number as sufficient to drive liquid layering (to avoid confounding the effects of arm number with the total free energy of interaction of each nanostar with the bulk given by the valence).

FIG. 7B shows the related experimental data. Strands for both nanostars were mixed (to a final concentration of 40 uM for each of 8 strands), 0.5M NaCl was added, and the mixture was heated to 55° C. for 30 minutes before being cooled slowly to 25° C. at −0.5° C./min. This resulted in the homogenously fluorescent tube at left. After centrifugation at 20,000×g for 1 hour at room temperature, the two condensates have clearly separated, according to their arm number (4 and 6 from top to bottom; note that there is a dark gas phase layer on top) as shown in FIG. 7 Panel C.

Other approaches are possible as will be understood by a skilled person upon reading of the present disclosure.

An example is provided in FIG. 9 which shows separation of nanostars into 3 layers based on the average number of arms, holding other structural properties roughly constant, using 4-armed stars (NS_{A}), a 50-50 mix of 4 and 8-armed nanostars (NS_{b}), and 8-armed nanostars (NS_{c}). FIG. 9 shows a principle very similar to FIG. 7. The top layer has an average of 4 arms, the middle layer an average of 6 arms, and the bottom layer an average of 8 arms. The top and bottom layers have trivial averages as they are comprised of a single nanostar each.

FIG. 9 demonstrates that condensates can be layered on the principle of varying the average number of arms, while leaving other structural features roughly constant. Three layers were designed according to the schematics at the top of FIG. 9. Arm lengths were all 20 nucleotides. The top layer was designed to be comprised of 100% of the 4-arm nanostar NS_{A} (4 strands each at 40 uM, FAM-tagged) with the sticky end-CGATCG-′3. Thus this layer had a density corresponding to an average arm number of 4. The middle layer was designed to be a 50%-50% mix of the 4-arm star NS_{b-4} (4 strands each at 20 uM, Cy3-tagged) and the 8-arm star NS_{b-8} (8 strands each at 20 uM, Cy3-tagged) both with the sticky end-GCATGC-3′. This mix of nanostars was intended to have an average arm length of 6. The bottom layer was design to be comprised of 100% of the 8 arm nanostar NS_{c} 8 (8 strands each at 20 uM, Cy5-tagged) with the sticky end-CACGTG-3′. Thus this layer had a density corresponding to an average arm number of 8. Note that single A flexible linkers were used for all sticky ends but are not diagrammed. Strands for all 4 nanostars were mixed, 0.5M NaCl was added, and the mixture was heated to 55° C. for 30 minutes before being cooled slowly to 25° C. at −0.5° C./min over 1 hour. This simple annealing protocol (a hold at 55° C., and a 1-hour ramp to room temperature) was used to form the nanostars. The image at left shows that simple annealing was not sufficient to separate these nanostars. Nanostars for all three designed layers were distributed uniformly within the tube. After centrifugation at 20,000×g for 1 hour at room temperature, all three condensates have clearly separated, according to their average arm number (4, 6, and 8 from top to bottom). Note that the condensate interfaces are not flat because the centrifuge used puts the microcentrifuge tubes at a tilt. Note also that the 8-armed nanostars in this experiment each have 4 blunt arms and 4 active sticky-end bearing arms. Blunt-ends were introduced into these 8-armed stars to enable us to keep the thermodynamics of the 8-armed nanostars similar to those of the 4-armed stars, and isolate average arm number as sufficient to drive liquid layering (to avoid confounding the effects of arm number with the total free energy of interaction of each nanostar with the bulk given by the valence).

A further approach is shown in FIG. 8 Panel B which exemplifies a totally different layering principle than FIG. 7 or FIG. 9. In particular, FIG. 8B demonstrates that condensates can be layered (to form 3 liquid layers) on the principle of varying the strength of the sticky ends (the interaction domains), while leaving other structural features roughly constant. It is supported by the fundamental trend observed for sticky end strength in FIG. 6. For approach illustrated in FIG. 8B, 4-arm, 24 nucleotide arm length condensate monomers were designed for four layers having ΔG of −3.5 (red, A), −4.1 (green, B), −9.2 (yellow, C) and −10.6 (orange, D) kcal/mol. Three of the four nanostars condensed, while the nanostar with the weakest sticky end strength (A, red) remained dispersed in the gas phase. This experiment demonstrates that ΔΔG as small as 1.4 kcal/mol (between layers C and D) can be used to provide robust layering.

In the experiments illustrated in FIG. 8 Panel B, each nanostar was at 41.6 uM, in 1× TE, pH 7.5, 0.5M NaCl. Thermal conditions were 1 hour hold at 60, followed by a ramp from 60° C. to 20° C. at 0.1 C/min. Centrifugation conditions were 40,000×g, 15 min spin in a 45-degree rotor at 20° C. followed by a second 3 min spin in a 90 degree rotor, 11,000×g at 20° C. (to flatten the layers relative to the tube axis). All tubes were 0.2 mL Corning PCR tubes. Using these small, thin-walled PCR tubes is important so that good fluorescence pictures can be taken using a fluorescent lamp at 302 nm.

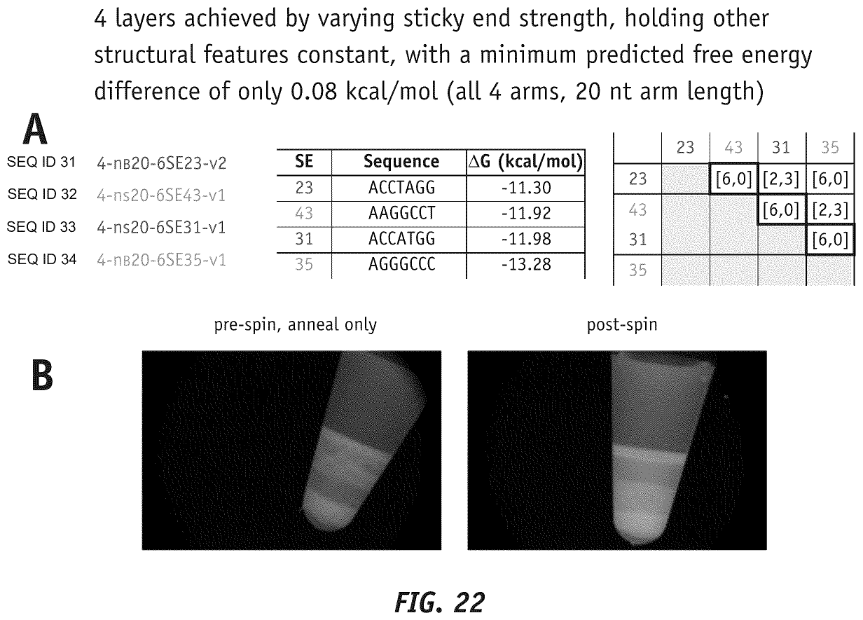

FIG. 22 shows another example similar to FIG. 8B, demonstrating layering by varying sticky end strength, and leaving other variables constant, this time to achieve 4 layers. The predicted ΔG for the 4 layers went from −11.3 (red), to 11.92 (green), to −11.98 (red), to −13.28 (green), kcal/mol). The Number of arms is constant (4) and the length of the arms is constant (20 nucleotides). Here the minimum ΔΔG is only 0.08 kcal, but as this is a single example, and ΔG predictions with different software packages easily vary by more than 0.08 kcal, a more conservative ΔΔG (e.g. 1 kcal or 0.5 kcal) should be used for engineering. Checking on the sticky end strength using different software packages, and measuring the difference in predicted sticky end strength should give a minimum ΔΔG that should be attempted.

The experimental parameter for the experiments of FIG. 22 were the following. Sample conditions Nanostar concentration: ranges from 36.4 uM-62.4 uM for each nanostar, to attempt to get volumes of condensate that larger volumes for the upper layers because of increasing tube diameter. 2% fluorescence for each nanostar Buffer: 1xTE, 0.5M NaCl Annealing conditions: 1 hr hold at 60° C., followed by −0.05 C/min ramp down to 20° C. Spin conditions 20 min, 20 C, 11710rcf, 90° rotor. Imaging conditions. UV transilluminator, 302 nm.

Different features of the nanostar can be operated in combination to provide additional approaches to layering. For example, the principle demonstrated for FIG. 9 is robust to large changes in experimental protocol. A set of condensates that layer under one experimental protocol tend to be easily layered using many experimental protocols.

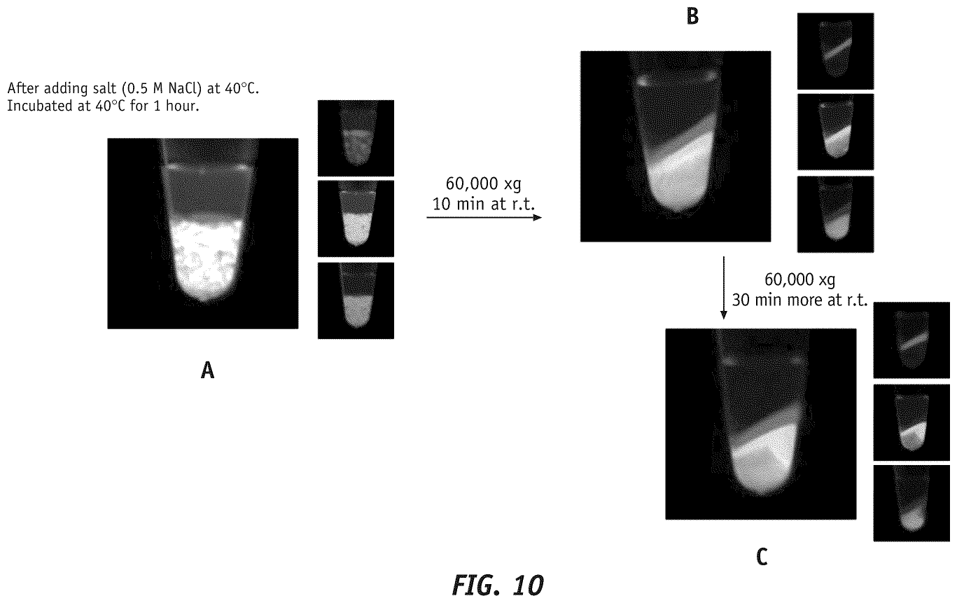

Also FIG. 10 shows separation of the same nanostars as in FIG. 9 into 3 layers based on the average number of arms, using a constant lower temperature nanostar formation protocol (no anneal), and shorter, higher speed centrifugation. In particular, FIG. 10 shows separation of the same nanostars as in FIG. 9 into 3 layers based on the average number of arms, using a constant lower temperature nanostar formation protocol (no anneal), and shorter, higher speed centrifugation.

FIG. 10 shows the robustness of layering of the nanostars based on arm length to a different annealing and centrifugation protocol. In particular because the condensates never go above 40° C., this experiment shows that these particular condensates should be compatible with most proteins (where proteins are often handled at 37° C.). Preparation of the strand mixes were identical except that 0.5 M NaCl was added at room temperature for FIG. 9, but for FIG. 10, salt was added at the hold temperature of 40° C. Where FIG. 9 uses a higher temperature hold, at 55° C. for 30 minutes, FIG. 10 uses a lower temperature hold of 40° C. for 1 hour. FIG. 10 Panel A shows that this temperature hold was not sufficient for layering. After a shorter centrifugation (10 minutes at 60,000×g at room temperature) the quality of the layering in FIG. 10 Panel BB is similar or better to that in FIG. 9 (right). Further 60,000×g centrifugation for 30 more minutes at room temperature does not improve the quality of the liquid layer separation. In fact an artifact often seen in tilted rotor tubes has appeared, the green middle layer can be seen to be slumped down the side of the tube, intruding into the pink layer; this artifact disappears during centrifugation with a swinging bucket rotor.

FIG. 10 shows the robustness of layering of the nanostars based on arm length to a different annealing and centrifugation protocol. In particular because the condensates never go above 40° C., this experiment shows that these particular condensates should be compatible with most proteins (where proteins are often handled at 37° C.). Preparation of the strand mixes were identical except that 0.5 M NaCl was added at room temperature for FIG. 9, but for FIG. 10, salt was added at the hold temperature of 40° C. Where FIG. 9 uses a higher temperature hold, at 55° C. for 30 minutes, FIG. 10 uses a lower temperature hold of 40° C. for 1 hour. FIG. 10A shows that this temperature hold was not sufficient for layering. After a shorter centrifugation (10 minutes at 60,000×g at room temperature) the quality of the layering in FIG. 10B is similar or better to that in FIG. 9 (right). Further 60,000×g centrifugation for 30 more minutes at room temperature does not improve the quality of the liquid layer separation. In fact an artifact often seen in tilted rotor tubes has appeared, the green middle layer can be seen to be slumped down the side of the tube, intruding into the pink layer; this artifact disappears during centrifugation with a swinging bucket rotor.

Therefore, FIG. 10 is one proof of principle that layering can be achieved at a relatively low constant temperature 40° C., consistent with many proteins being natured, many ligands being natured, and many ligand-protein interactions being stable.

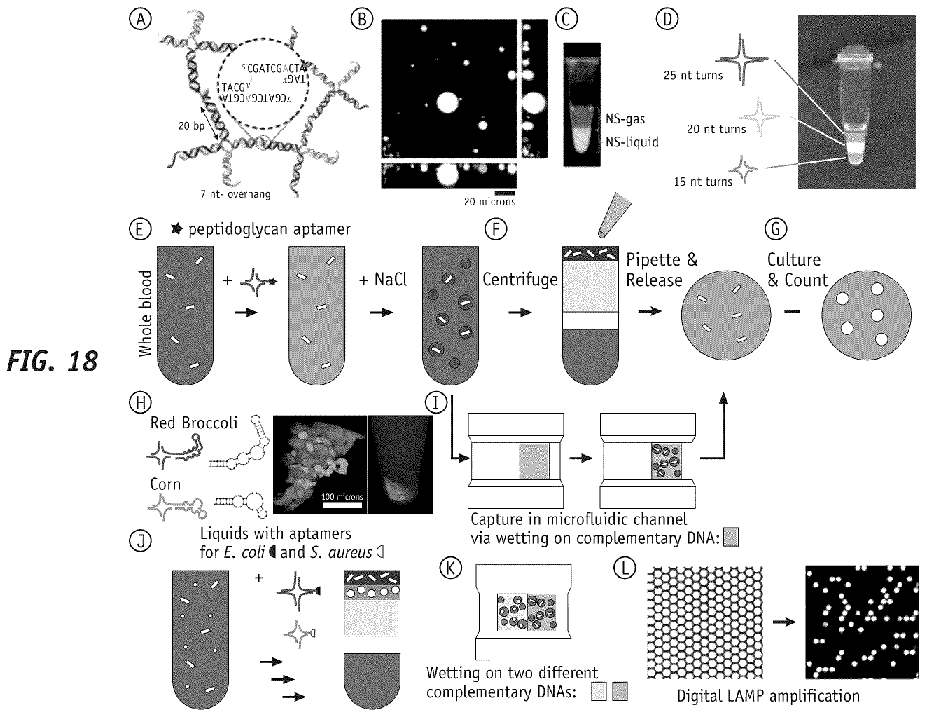

Additional exemplary approaches are illustrated in FIGS. 13-17 which show a more extreme example of constant temperature phase separation for a DNA condensate (at room temperature). for a single layer. FIG. 18d shows liquid layering based on changes to arm length, holding other structural features constant. See Example 12 for more information on this experiment.

A skilled person will understand that it is possible to change more than one structural feature at the same time, and achieve predictable layering in a reliable way (without additional experiments) if the density trends for the more than one structural feature are all simultaneously respected.

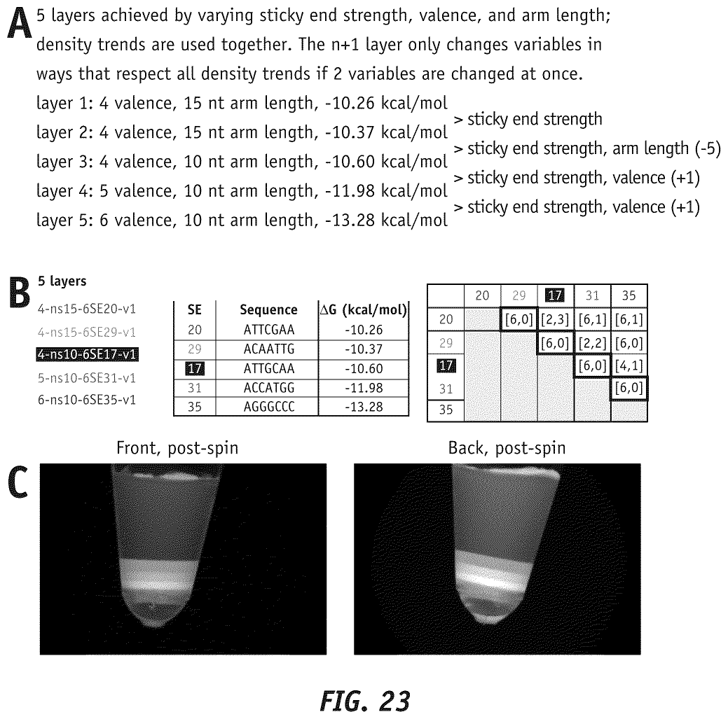

FIG. 23 demonstrates this approach to create a 5-layered DNA condensate. Observe that when sticky end strength and arm length are both changed (from layer 2 to 3), they are both changed in a way that increases density. Observe that when sticky end strength and valence are both changed (from layer 3 to 4, and from layer 4 to 5), they are both changed in a way that increases density, according to known trends.

The experimental parameters for FIG. 23 are the following Sample conditions: Nanostar concentration: 60 uM each. 2% fluorescence for each nanostar (except for 6-ns10-6SE35-v1, which had no fluorophore added) Buffer: 1xTE, 0.5M NaCl Annealing conditions: 1 hr hold at 60° C., followed by −0.05 C/min ramp down to 20° C. Spin conditions: 30 min, 20 C, 11710 rcf, 90° rotor Imaging conditions: UV transilluminator, 302 nm.

The layered condensate structures discussed in this example were constructed based on trends for which the density of the individual nanostars was not necessary to know before layering. Where it is necessary to do so, density of the condensate forming each layer can be calculated and/or determined with web-bench techniques as will be understood by a skilled person upon reading of the present disclosure.

An exemplary method to determine density of a nucleic acid condensate through, detection of the concentration of a nucleic acid monomer is provided by electronic absorption spectroscopy methods well-known to those skilled in the art. For example, a 1-2 uL sample of an individual condensate can be removed by pipette and transferred to a microcuvette and investigated by UV-visible spectroscopy using a suitable instrument such as a Thermo Scientific Nanostar 1000, a DeNovix DS-11 or an Unchained Labs Lunatic. The concentration of nucleic acid in the condensate can be determined by measuring the absorbance of the sample at 260 nm and applying the Beer-Lambert law, with an absorbance (A) of 1 corresponding to a concentration of 40 μg/mL nucleic acid. Concentration for a specific sample may thus be calculated directly from the measured absorbance proportionally, as long as the absorbance measured is in the linear regime (A=0.2-1.0); if not, the sample can be diluted or concentrated accordingly to meet this requirement, and the dilution factor (DF) used in the calculation to obtain the original sample nucleic acid concentration according to the equation Concentration (ug/mL)=A*40*DF (1).

In an alternative method, the concentration of a nucleic acid condensate can be determined by fluorescence spectroscopy methods. For example, a 1-2 uL sample of an individual layer may be removed by pipette and transferred to a microcuvette or microwell plate and treated with a fluorescent dye used for assaying nucleic acids (e.g. ethidium bromide, or QuantiFluor dyes (Promega) in accordance with the assay protocols. The assay may be performed on the sample using a fluorimeter such as a DeNovix DS-11 or a Promega Quantus, and the nucleic acid concentration quantified by comparison with a fluorescence intensity calibration curve. As above, the value thus determined for nucleic acid concentration corresponds directly to the nucleic acid condensate layer density.

Additionally, if large samples of condensate are available (e.g., >10 mL), measurement of the nucleic acid condensate layer viscosity using a Ubbelohde viscometer and comparison to known nucleic acid standard viscosities provides the condensate concentration.

### Example 5: Separation and Detection of Biomolecular Structure Via Phase Separation of Condensate into a Layered Structure

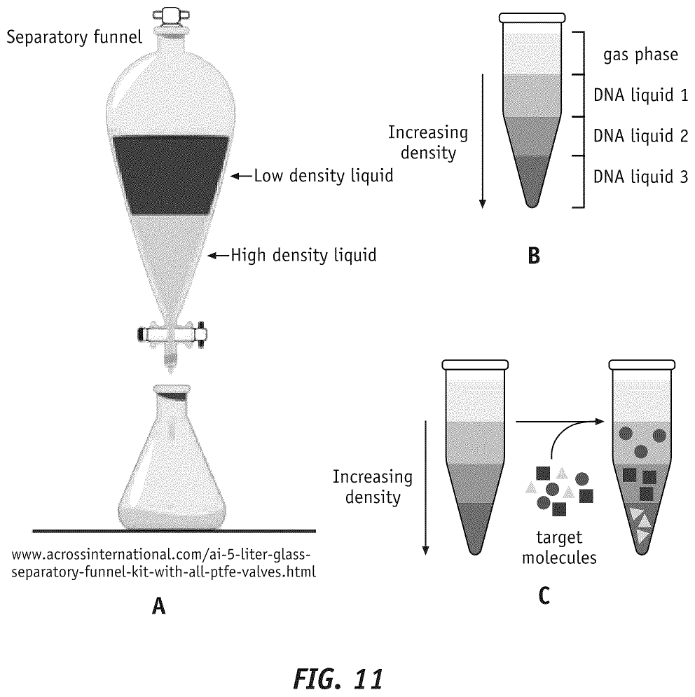

A layered structure formed by nucleic Acid condensate engineered to have set density can be used to separate mixtures. FIG. 11 gives schematics showing the analogy between the use of a classic separatory funnel and the use of layered DNA condensates.

In particular, FIG. 11 shows the analogy between the use of phase separation in a classic separatory funnel to separate molecules, and the use of layered DNA condensates to separate molecules. In a classic separation using separatory funnel (FIG. 11A), a pair of immiscible liquids are used to separate a desired compound from undesired molecules, typically starting materials or side products from a chemical reaction. The reaction mixture, dissolved into one solvent, is mixed with a second immiscible solvent of a different density within the separatory funnel. The desired compound ends up in one of the two layers based on its chemical character (e.g. polar or nonpolar), and the compound is recovered via physical removal of the layer of liquid which contains the desired compound, and then removal of the liquid (via rotary evaporation or recrystallization). FIG. 11B shows a microcentrifuge tube containing a layered structure of three immiscible DNA liquid condensates of increasing density, along with an aqueous gas phase on top. FIG. 11C shows separation of a mixture of three compounds (squares, circles or triangles) using the DNA condensate. Here the affinity of each compound for each DNA layer is not set by the compounds chemical character, but rather the affinity of the compound for a ligand within the DNA condensate (e.g. an aptamer for a protein).

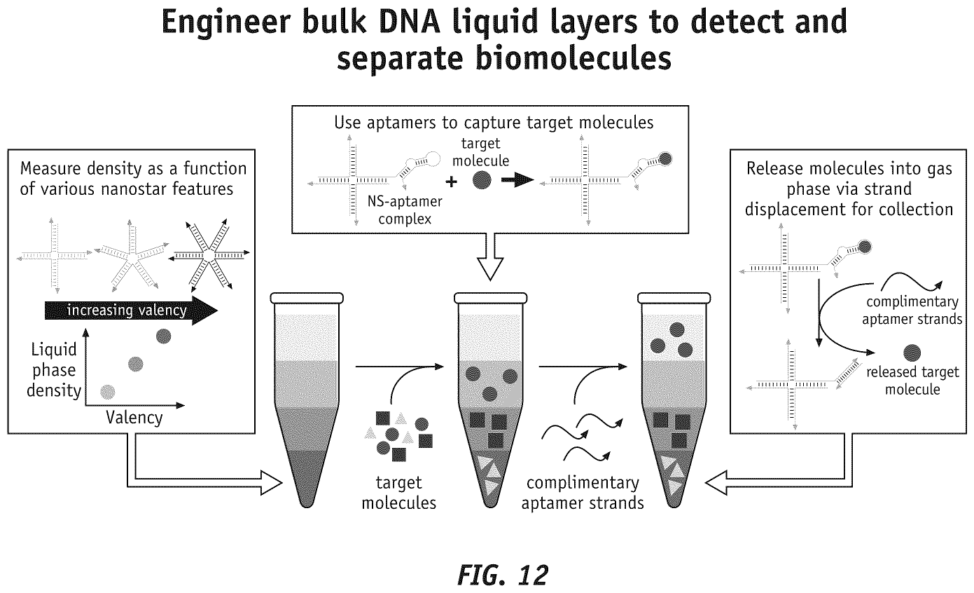

Bulk DNA liquid layers can also be engineered to separate and detect biomolecular targets. FIG. 12 gives the basic workflow for the separation of molecules using a layered DNA condensate.

FIG. 12 shows schematics the basic mechanism by which target molecules bind to their corresponding layers in a layered set of DNA condensates and the mechanism whereby they are released. Here a folded nucleic acid aptamer (RNA or DNA) covalently attached to one of the nanostar arm strands, provides a mechanism for the binding of a specific target molecule (purple), when the mixture of target molecules is mixed by pipetting into the layered condensate (which will destroy the layering, not depicted). At this point, targets should be bound to condensate. After relayering (not depicted) targets are sorted by condensate layer. To selectively release a target from a particular layer, a oligonucleotide release agent is mixed into the layered condensate (which will destroy layering but release the target through strand displacement). Upon relayering (not shown) the target molecule can be collected from the supernatant gas phase. As described, this protocol is appropriate for any entity for which one can make an aptamer, including small molecules, proteins, and cells. It is noted that the sequence specific separation of target nucleic acids can be achieved with exactly this workflow, where the aptamer is replaced by a simple DNA strand, which is complementary for a length of 20 nucleotides somewhere on the target nucleic acid.

### Example 6: Capture and Release of Streptavidin Via Phase Separation of Condensate into a Monolayer Structure

A nucleic acid condensate was engineered to capture and release streptavidin as a proof of principle of use of polynucleotide condensate to separate mixtures.

FIG. 13A shows the cartoon schematic for an exemplary method capturing a target molecule in a layered condensate. FIG. 13B shows the control experiment when streptavidin is mixed with a single-layer condensate having no streptavidin aptamer, and the mixture is layered by centrifugation. The streptavidin is excluded from the condensate and remains in the gas layer. In general, targets having no intrinsic affinity for DNA will be excluded from the condensate.

FIG. 13C show the data for the experiments generated using a protocol: Nanostars having 4 arms, 20 nucleotide arm length were used. 83.2 uM of nanostars were used but only 10% had streptavidin aptamers. Nanostars were not annealed, their strands were mixed without salt, and 1.8 uM streptavidin was added to the nanostars before the addition of 0.5 M salt. This mixture was then incubated in the fridge at 4 for 1 hour and spun at 40,000×g, 15 min, 23° C. The protocol for FIG. 13B (the condensate having no aptamers) was the same for that with aptamers except the input concentration of nanostars was higher (138 uM) and so a larger volume of condensate formed.

The results reporting in FIG. 13 Panel C show a condensate which has an attached DNA aptamer for streptavidin. The cartoon shows that the attached streptavidin aptamer has been attached to the condensate via a linker. One of the arms of the monomer has been extended with a linker domain, and a corresponding complementary extension on the aptamer attaches it to the condensate monomer. This scheme is modular, so that a different aptamer, bearing the same single stranded extension, could be easily used instead. The experiment shows that after mixing the streptavidin with the condensate, and relayering, the bulk of the streptavidin is confined to the condensate at the bottom of the tube.

The streptavidin binding nanostar used in the experiments of FIG. 13 has the following exemplary features: The aptamer sequence used is a modified version of the streptavidin aptamer is St-2-1, which is from FIG. 4 of (Tao Bing et al, “Conservative secondary structure motif of streptavidin-binding aptamers generated by different laboratories”, Bioorganic & Medicinal Chemistry Volume 18, Issue 5, 1 Mar. 2010, Pages 1798-1805).

The aptamer sequence to include an extra GC closing base pairs at the ends to help promote formation of the stem-loop structure, i.e. 5′ GC-(St-2-1)-GC 3′ or 5′ GC-ATTGACCGCTGTGTGACGCAACACTCAAT-GC 3′ (SEQ ID 13).

The nanostar used for the streptavidin experiments is 4-ns20-6SE49-v3; here are the sequences for that nanostar:

SEQ ID NO 5:
CTACTATGGCGGGTGATAAAAACGGGAAGAGCATGCCCATCCACGATCG;
SEQ ID NO 6:
GGATGGGCATGCTCTTCCCGAACTCAACTGCCTGGTGATACGACGATCG;
SEQ ID NO 7:
CGTATCACCAGGCAGTTGAGAACATGCGAGGGTCCAATACCGACGATCG;
SEQ ID NO 8:
CGGTATTGGACCCTCGCATGAATTTATCACCCGCCATAGTAGACGATCG

To modify the nanostar to include the aptamer, we replaced a percentage (5-10%) of SEQ ID NO 8 with the strand stran4_R2tag below, which has a “targeting domain” added on its 5′ end: CGATATGTCTACTTAACATG-AACGGTATTGGACCCTCGCATGAATTTATCACCCGCCATAGTAGACGATCG

The aptamer strand (ligand) had a complementary tag (to bind the targeting domain):

R2tag_St-2-1
(5' aptamer-toehold-20mer_tag_complement 3'):
(SEQ ID 14)
GCATTGACCGCTGTGTGACGCAACACTCAATGC-
TTAGG-CATGTTAAGTAGACATATCG

There is a 5nt toehold between the complementary 20-mer tag and aptamer to allow for displacement using displacer strand disp_St-2-1 below:

disp_St-2-1
(5' aptamer_complement-toehold_complement 3')
(SEQ ID 15)
CCTAAGCATTGAGTGTTGCGTCACACAGCGGTC-AATGC

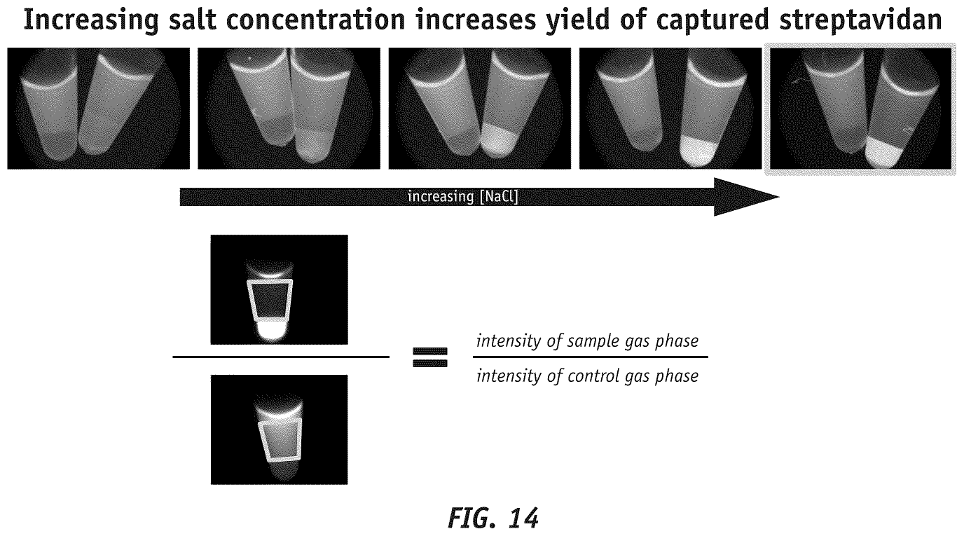

Additional parameters can be operated to control the binding of a condensate monomer to a ligand. For examples, FIG. 14 shows how the salt dependence of streptavidin capture in a liquid layer is measured. The results illustrated in FIG. 14 were generated using a protocol: Nanostars having 4 arms, 20 nucleotide arm length were used. All tubes had 125 uM nanostars. For the tubes that had aptamers (the righthand tube in each image) 5% had streptavidin aptamers. Nanostars were not annealed, their strands were mixed without salt, and 0.1 mg/mL streptavidin was added to the nanostars before the addition of salt (100 mM, 250 mM, 500 mM, 750 mM, or 1M NaCl). Then all tubes were incubated at 20° C. for 1 hour. Tubes were then first spun 15 minutes at 40,000×g, at 20° C. with a 45-degree angled rotor followed by a second 3 minute spin at 11,000×g in a 90-degree spinning bucket rotor to flatten the condensate.

FIG. 14, top shows a series of five images wherein the concentration of sodium was increased (100 mM, 250 mM, 500 mM, 750 mM, and 1M NaCl). In each image the left control shows the non-interaction of streptavidin with a condensate having no aptamer. The right tube shows the interaction of streptavidin with a condensate bearing streptavidin aptamer. The degree of streptavidin binding was measured by dividing the intensity of a trapezoidal region of the “sample” gas phase by the intensity of a trapezoidal region of the control tube gas phase, where the sample gas phase means the gas phase above a condensate bearing streptavidin aptamers. The intensity of the gas phase was measured because direct measurement of the intensity of the condensate at the bottom of the tube was made difficult by both the shape of the tube bottom and variability in the plastic manufacture at the bottom of the tube.

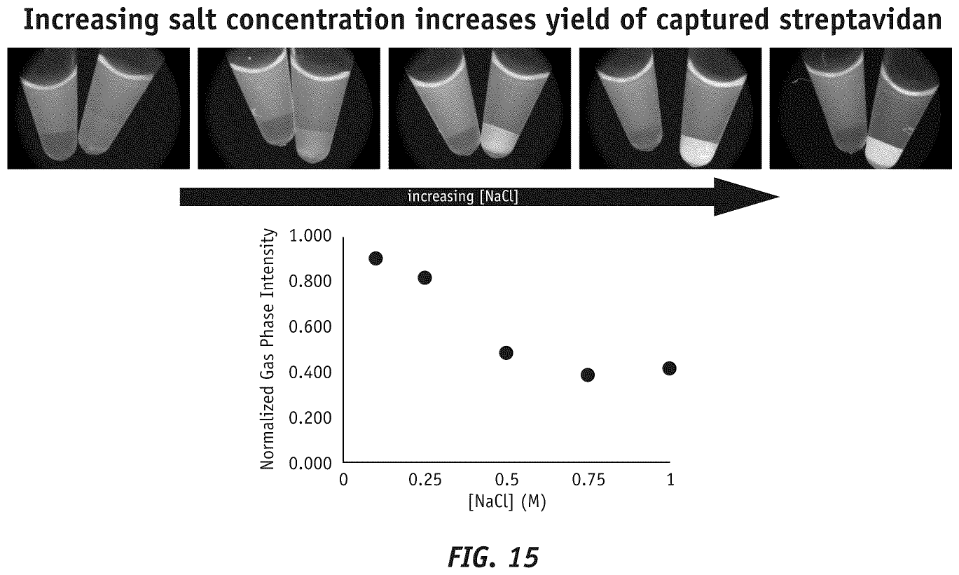

FIG. 15 shows that streptavidin capture by aptamers in a DNA condensate saturates at 750 mM Sodium. FIG. 15 was generated using the following protocol: Nanostars having 4 arms, 20 nucleotide arm length were used. All tubes had 125 uM nanostars. For the tubes that had aptamers (the righthand tube in each image) 5% had streptavidin aptamers. Nanostars were not annealed, their strands were mixed without salt, and 0.1 mg/mL streptavidin was added to the nanostars before the addition of salt (100 mM, 250 mM, 500 mM, 750 mM, or 1M NaCl). Then all tubes were incubated at 20° C. for 1 hour. Tubes were then first spun 15 minutes at 40,000×g, at 20° C. with a 45-degree angled rotor followed by a second 3-minute spin at 11,000×g in a 90-degree spinning bucket rotor to flatten the condensate.

The results illustrated in FIG. 15 show the features of streptavidin monomer binding at these conditions. In particular, FIG. 15, top shows a series of five images wherein the concentration of sodium was increased (100 mM, 250 mM, 500 mM, 750 mM, and 1M NaCl). In each image the left control shows the non-interaction of streptavidin with a condensate having no aptamer. The right tube shows the interaction of streptavidin with a condensate bearing streptavidin aptamer. The degree of streptavidin binding is measured as described in FIG. 14. Note that streptavidin binding to the correct liquid phase is anti-correlated with the remaining fluorescent streptavidin signal in the gas phase. Thus a low normalized gas phase intensity (normalized by the gas phase of the control tube) means a high amount of streptavidin binding. The measure is semiquantitative but it suggests that the binding of streptavidin to the condensate layer saturates at 750 mM NaCl at about 60% of the total streptavidin. This separation has not been optimized for the volume of condensate, whose binding capacity has not been measured. The overall trend for streptavidin binding can be explained by increased folding of the aptamer as NaCl is increased. Many aptamers become better binders of their cognate targets as salt is increased because the aptamer becomes better folded and a greater fraction of the aptamers assume a conformation that bind the target (the target binding state of the aptamer becomes thermodynamically favored, its Tm increases). Eventually, the trend often reverses, as the salt begins to screen some of the interaction of the aptamer with the target. A hint of this effect may be seen at the 1M NaCl data point in the graph which suggests a decrease to ˜55% binding of the streptavidin.

In embodiments herein described targets captured in a functionalized nucleic acid condensate of the disclosure can be released in a controlled fashion.

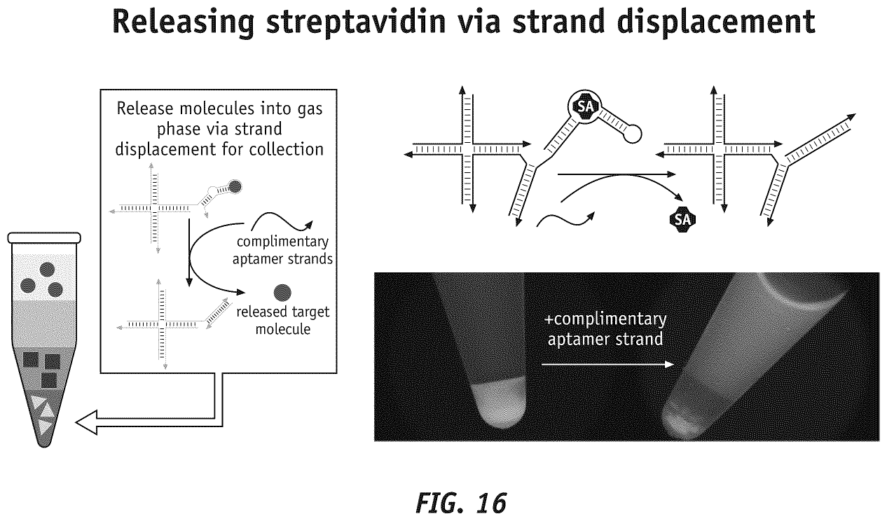

FIG. 16 shows proof of principle on condensate attaching a streptavidin, that addition of a release strand frees streptavidin into the gas phase. FIG. 16, left side shows a schematic of the addition of a release strand to the aptamer. The release strand is complementary to the full length of the aptamer, plus at least a 5 nucleotide single stranded toehold present on either the 5′ or 3′ end of the aptamer which serves an open binding site for the strand displacement process to initiate. Once initiated a random walk between the incoming release strand and the aptamer fold terminates in full opening of the aptamer to form a duplex and release of the target molecule. Such an oligonucleotide release strand enables a target to be released specifically, from a condensate liquid layer, without release of any other targets in any other layer. The right side of FIG. 16 shows an experimental implementation of this using streptavidin. Here the release strand was added to the condensate at left via pipetting. The condensate was then relayered by centrifugation and the bulk of the streptavidin was released back into the gas phase. Some streptavidin was retained in a small volume of condensate within the lower layer. Such impartial release is indicative of imperfect mixing of the release strand with the condensate, and can be avoided by more extensive mixing of the release strand into the condensate.

The experimental details used to generate the results shown in FIG. 16 are the following. To create the condensate in the lefthand tube, before streptavidin release, nanostars having 4 arms, 20 nucleotide arm length were used. 83.2 uM of nanostars were used but only 10% had streptavidin aptamers. Nanostars were not annealed, their strands were mixed without salt, and 1.8 uM streptavidin was added to the nanostars before the addition of 0.5 M salt. This mixture was then incubated in the fridge at 4 for 1 hour. Spun at 40,000×g, 15 min, 23° C. To release the streptavidin, displacing strand was added to a final concentration 13 uM. Immediately after addition, roughly 50% of the streptavidin was released. The tube pictured at right is the tube after it has been incubated in the fridge for week at 4° C.

### Example 7: Capture and Release of Streptavidin Via Phase Separation of Condensate into a Multilayer Structure

The results obtained in preceding example and showing the ability of nucleic acid condensate to capture and release streptavidin in a monolayer structure provides a proof of principle of use of polynucleotide condensate to separate mixtures in multilayer structure.

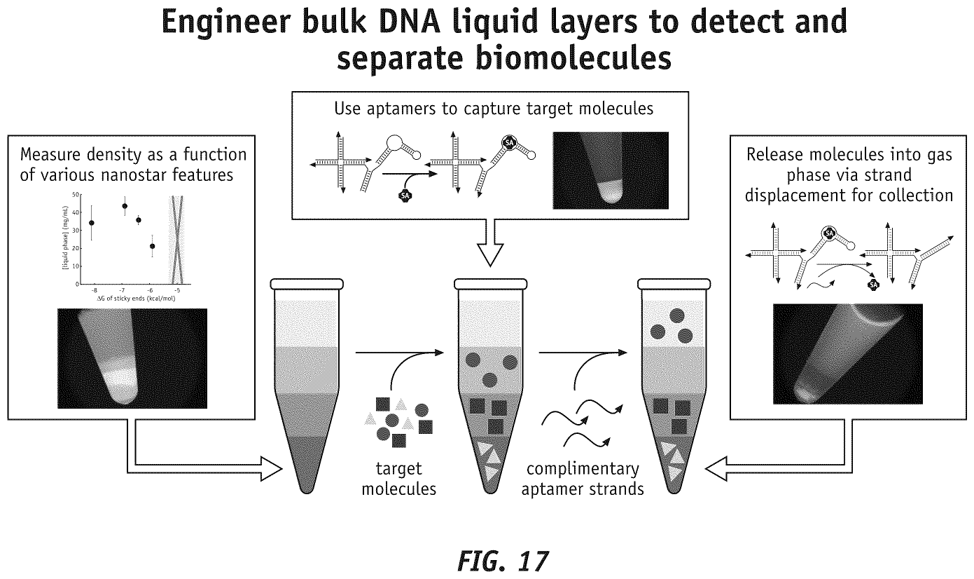

An exemplary approach is reported in FIG. 17 which shows schematic analogous to schematic FIG. 12, except for (1) it replaces the schematic layered condensate with an experimental data on a layered condensate based on arm length (from FIG. 8B), (2) it replaces a generic aptamer binding scheme (top center) with data on a streptavidin-binding condensate (from FIG. 13C and FIG. 16) and (3) it replaces a generic strand displacement schematic with data on sequence specific release of streptavidin from a streptavidin-binding condensate (from FIG. 16). Thus it demonstrates that all the schematics for the separate elements presented in FIG. 12 can be realized in experimental systems, which can be combined by one of sufficient skill in the art to realize a multilayer system for the capture and release of multiple targets.

### Example 8; Artificial Nucleic Acid Condensates for Rapid Concentration of Bacteria From Blood

Sepsis, the body's inflammatory response to a bacterial infection of the blood, afflicts 30 million people worldwide and results in 6 million deaths every year [33]. In the U.S. sepsis is the single most expensive treated condition, with costs in excess of $20 billion per year [34]. During infection, the levels of bacteria in the blood are typically extremely low (1 to 100 CFU/ml) [35], which renders diagnosis difficult. New methods for the efficient concentration and recovery of bacteria from whole blood are direly needed.

Over the last decade, RNA and protein liquid-like condensates have been found to play a role in an ever-increasing number of biological phenomena, where they serve as dynamic, membraneless organelles that localize molecules in space and time [35]. [36] [37] [2] [38].

Taking these natural condensates as inspiration, biological engineers have created artificial condensates that can perform a range of tasks, from single condensates that can be programmably divided into two condensates [3], to the cellular expression of proteins with unnatural amino acids within an artificial organelle [39]. The concentration of other molecules is a common occurrence in biological condensates, e.g. the localization of tubulin to a pericentrosomal condensate during cell division [40]. In artificial DNA condensates (FIG. 18a-c, [1], [4]), we have demonstrated the concentration of a target molecule by adding a binding domain for the target to the monomer which forms the condensate (FIG. 13-17). Binding domains for arbitrary targets can be generated through in vitro molecular evolution (e.g. SELEX [41]. [42]). Target-binding DNA and RNA sequences discovered this way are known as aptamers.

In this example, we have provided a rapid and specific method of concentrating bacteria from blood, without the complexities and costs of approaches that use aptamer or protein-coated nanoparticles [43], [44]. It has the advantage that immiscible DNA condensates allow for multiplexing in both centrifugal and microfabricated settings (FIG. 18j, 18k). And it offers total integration of concentration and bacterial enumeration in a single microfluidic platform that combines wetting based separation (FIG. 18i,k) with existing technologies (FIG. 18l, [5], [45]) for digital loop-mediated isothermal amplification (LAMP).

In one embodiment, artificial DNA or RNA condensates whose monomers bear aptamers with affinity for bacteria are used. Mixed with blood samples, these condensates concentrate bacteria by a large factor (60-500×). The condensates are then separated from blood by low-speed centrifugation or in microfabricated devices, and the bacteria are released for further analysis such as sequencing or bacterial enumeration [48].

Artificial DNA condensates [1] are typically constructed from four DNA strands that form multi-armed “nanostar” monomers whose sticky ends have six nucleotides of self-complementarity (FIG. 18a). Upon addition of monovalent salt, nanostars condense to form 5-20 micron droplets (FIG. 18b) that can be coalesced into a bulk liquid with low speed centrifugation (FIG. 18c, e.g. 100 min at 3,000×g, or 20 min at 18,000×g). We have synthesized a series of three immiscible DNA condensates (FIG. 18d) whose monomers' difference in size (arm length) creates large enough differences in density that they can be separated by low-speed centrifugation.

To handle infections with unknown pathogens, condensates (FIG. 18e) bearing aptamers that have affinity to a broad class of bacteria [49, 50] are used. For example, aptamers raised against the peptidoglycan wall of gram negative bacteria are able to bind to a broad range of both gram negative and gram positive bacteria [52].

Condensate monomers mixed at high concentration (1-10 micromolar) with whole blood react and coat bacteria within a minute. Addition of concentrated salt (˜250 millimolar) induces condensation of droplets, which entrains the bacteria. Centrifugation (FIG. 18f) coalesces liquid droplets in a layer at the top of the plasma blood fraction where it can be pipetted off, and the bacteria are released, cultured, and counted (FIG. 18g).

The bacteria (density 1.1 g/ml) would normally sediment with white blood cells (1.08 g/ml, [53] at the bottom of the tube with sufficiently strong centrifugation. However, adhesion between thousands [54] of aptamers and each bacterial cell will partition the bacteria with the DNA condensate (1.01-1.04 g/ml) just above the plasma (1.025 g/ml) or just below it (not shown) on top of the white blood cells (1.08 g/ml). This is similar to the case of DNA extraction wherein DNA, whose density is 1.7 g/ml, does not sediment below chloroform of density 1.49 g/ml, and instead partitions on top in a ˜1.0 g/ml aqueous layer to which it is attracted. The small amount of DNA dissolved in the aqueous layer does not disturb the density of the aqueous layer.

The same argument applies to bacteria in the DNA condensate. Note that even given a 1000× concentration of bacteria in the DNA liquid layer (higher than the 500× available to our method) there will only be 100,000 CFU/mL (roughly 100,000 bacteria/mL). This corresponds to 100 bacteria in one microliter, a cube 1 millimeter on each side. The average distance between 1 micron bacteria will be ˜0.2 millimeters. The volume fraction of the 100 bacteria will be 1E-9. At such a low concentration and low volume fraction the bacteria in the DNA condensate will not affect its density and change its layering properties.

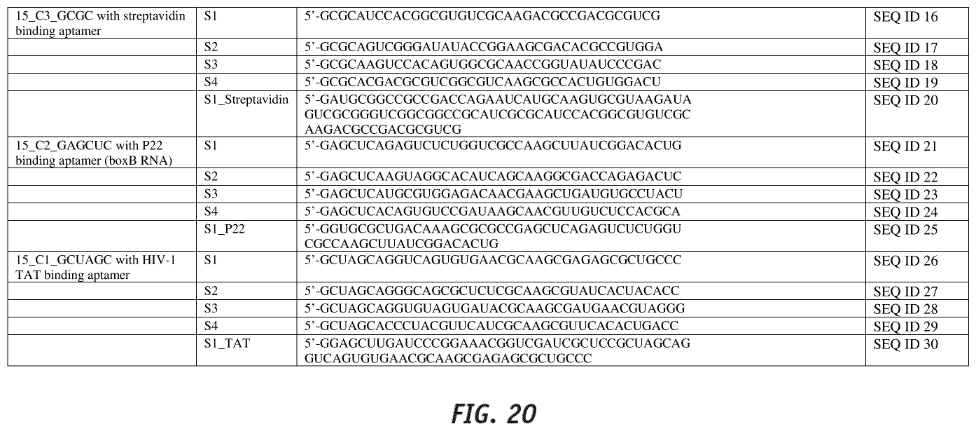

Alternatively, droplets are separated from whole blood in a microfluidic apparatus (FIG. 20i) via capture on a patch of complementary DNAs [55] In either case, bacteria are conveniently freed at room temperature by dissolving the condensate using strand displacement or a reduction in salt concentration. We calculate that with an input of 20 microliters ($1 worth) of condensate monomer our system will achieve 60-fold concentration of bacteria from 1 ml of blood with a maximum of 85% recovery yield, or a 500-fold concentration from 10 ml of blood with a maximum of 40% recovery.

In cases where a specific pathogen is suspected, or two specific pathogens are being discriminated, aptamers raised against defined bacterial species may be used. We have explored the effect of aptamers grafted to nanostars, in the context of RNA condensates labelled with the fluorescent light-up RNA aptamers Corn and Red Broccoli FIG. 18h, microscopically and macroscopically phase-separated) and have shown that pairs of distinct aptamers can work in tandem.

For decades Staphylococcus aureus and Escherichia coli have traded places as the top two leading causes of sepsis [35], [58], [59]. Thus in one embodiment, condensates with two different aptamers specific to E. Coli [60] and S. aureus [54], are able to differentiate between different species (FIG. 18j).

In another embodiment which does not require aptamers, DNA-protein conjugation techniques [61] [62] [63] techniques are used to fuse bacteria-binding proteins to condensate monomers.

### Example 9: Approach to Engineer Condensate Monomers

In choosing the structural features of a condensate monomer, a first question of which condensate monomer architectures (including choices of all structural features) are highly likely, if not deterministically to robustly phase separate as a liquid condensate rather either failing to phase separate (remaining in the diffuse state), or phase separating as gel. Below we give guidance for a variety of nanostar structural features, based on the large number of nanostars present in the literature and our own experiments.

Before giving specific numeral guidance for the various values of structural features, a skilled person consider the relationship between two features, arm length and interaction domain strength, that are generally important for the successful creation of a liquid nanostar condensate. An important design consideration is that there is a separation of formation conditions (for temperature, or salt concentration) for the formation of the condensate monomer and the liquid condensate. If the separation of the formation (for example with respect to free energies and thus melting temperature, or salt formation concentration) of the condensate monomers (interactions between strands of the monomers) from the formation of the liquid condensate (interactions between the monomers) is insufficient, and the resulting material is not that desired (for example it might be a disordered network of strands with no clearly defined condensate monomers, and which does not act as a liquid condensate), then the material may perform poorly with respect to layering or separation.

One way to ensure that condensate monomers form first, before the liquid phase condenses as experimental conditions are changed (either temperature is decreased or salt is added) is to make the interaction domains weak relative to the arm domains. A good heuristic for condensate monomer design is either that (1) that the predicted free energy ΔG of association for the interaction domain (as a bimolecular pair) is less than 50% of the ΔG of association for an individual pair of arm domains (e.g. a 20 nucleotide duplex for a condensate monomer with 20 nucleotide arms) or (2) the predicted melting temperature of the interaction domain is 10 C less than that of an individual pair of arm domains. In lieu of predicted values for free energies or melting temperatures, empirical values from the literature may be used, as long as the experimental conditions under which the values are measured are similar, or the values can be extrapolated to those which would be measured under similar conditions.

With respect to guidance on arm lengths, arm lengths of condensate monomers can be freely varied from a length of 10 to 50 nucleotides. The original DNA liquid condensate paper by Biffi et al. used arm lengths of 20 nucleotides, [1]. Here we demonstrate arm lengths from 10 nucleotides (FIG. 23) to 25 nucleotides (FIG. 18d). Arm lengths below 10 nucleotides run the risk of creating arms that are too weak relative to the strength of the interaction domains. In some embodiments the use of high GC content in arms less than 10 nucleotides long in combination with weak interaction domains (e.g. AT sticky ends of length 2) will provide sufficient separation of free energies that well defined condensate monomers and predicted condensates form. Arm lengths above 50 become expensive to synthesize; they imply overall strand lengths of 100 or greater, that are above the limit for better purity guarantees and lower costs for most DNA synthesis providers. Newer technologies for DNA synthesis may enable the practical synthesis of longer strands, and in some embodiments condensates with arm lengths above 50 may be used to create liquid condensates.

With respect to guidance on arm number, arm number can be freely varied between three and twelve. A minimum of three arms are needed to provide the three valences necessary for making a networked liquid condensate. Two arms is too small as only linear, not branching structures can be created from two-armed monomers. In the original DNA liquid condensate paper by Biffi et al [1], three and four arm condensates are demonstrated. Eight arm condensate monomers which make liquid condensates are demonstrated here in FIG. 9. Twelve armed junctions suitable as the basis for twelve armed condensate monomers have been demonstrated in Wang & Seeman (Wang & Seeman, “Assembly and characterization of 8-arm and 12-arm DNA branched junctions” J Am Chem Soc. 2007 Jul. 4; 129(26):8169-76. doi: 10.1021/ja0693441. Epub 2007 Jun. 12). More arms does mean more strands to synthesize, and greater costs, so for practical reasons, arm number should be minimized.

With respect to guidance on the flexible linkers at the junction between arms, the number of bases can be freely varied between zero and two nucleotides, with the identity of the nucleotides being chosen to avoid base pairing with other structures (e.g. A, AA, T, or TT). Two As are typically used in most works in the literature, following the original paper on DNA nanostars that make liquids, Biffi et al (Biffi et al, “Phase behavior and critical activated dynamics of limited-valence DNA nanostars”, PNAS 110 (39) 15633-15637). General guidance for a new and untested condensate monomer structure is to use AA for the flexible linker between the arms.

However liquid condensates have been formed with zero nucleotides (see e.g. Nguyen & Saleh, “Tuning phase and aging of DNA hydrogels through molecular design” Soft Matter, 2017, 13, 5421-5427) as long as flexible linkers (a single A nucleotide) were used for the interaction domain. Thymines have also been shown to be effective linkers, for example Sato et al (Sato et al, “Sequence-based engineering of dynamic functions of micrometer-sized DNA droplets” Science Advances, 3 Jun. 2020 Vol 6, Issue 23 DOI: 10.1126/sciadv.aba347). use TT as the flexible linkers for more than 10 effective condensate monomer designs. A general trend is that for fewer nucleotides in the flexible linker, condensates tend to form gels rather than liquids, and that for longer linkers the melting temperature will decrease (see e.g. Nguyen & Saleh, “Tuning phase and aging of DNA hydrogels through molecular design” Soft Matter, 2017, 13, 5421-5427). These tendencies can be offset by other structural features of the design (e.g. for longer linkers, more arms, or stronger interaction domains might be used). Thus in some embodiments it is possible that condensates with more than two nucleotides in the flexible linkers between arms are used.

With respect to guidance on the flexible linkers at the junctions with interaction domains, the structural feature is determined by the type of interaction domain that is used. For sticky ends interaction domains, most works in the literature use a single A nucleotide for flexibility, following the original paper on DNA nanostars, Biffi et al (Biffi et al, “Phase behavior and critical activated dynamics of limited-valence DNA nanostars”, PNAS 110 (39) 15633-15637). However, Sato et al (Sato et al, “Sequence-based engineering of dynamic functions of micrometer-sized DNA droplets” Science Advances, 3 Jun. 2020 Vol 6, Issue 23 DOI: 10.1126/sciadv.aba347) present more than 10 condensate monomer designs that make liquid condensates having a flexible linker for their sticky ends of length zero nucleotides (that is, no flexible linker). For blunt end interaction domains, then no additional flexible linker makes sense as the interaction domain is simply the end of a double helix.

With respect to guidance on the size of interaction domains, this depends on the type of interaction domain used. For sticky end interaction domains, lengths of 1-12 nucleotides are recommended. DNA condensate monomers demonstrated in Biffi et al (Biffi et al, “Phase behavior and critical activated dynamics of limited-valence DNA nanostars”, PNAS 110 (39) 15633-15637). have 6 nucleotide sticky ends for three-armed and four-armed nanostars, and so much of the literature condensate monomers follow this precedent. Sato et al (Sato et al, “Sequence-based engineering of dynamic functions of micrometer-sized DNA droplets” Science Advances, 3 Jun. 2020 Vol 6, Issue 23 DOI: 10.1126/sciadv.aba347) demonstrate DNA liquid condensates whose sticky ends have a range of 4 to 12 nucleotides. Stewart et al (Stewart et al “Modular RNA motifs for orthogonal phase separated compartments” Nature Communications volume 15, Article number: 6244 (2024) demonstrate successful RNA liquid condensates whose sticky ends have a range of 2 to 6 nucleotides on four-armed monomers. Single and double (1-2) nucleotide sticky ends are routinely used to assemble DNA origami (see e.g. Petersen et al, “Information-based autonomous reconfiguration in systems of interacting DNA nanostructures” Nature Communications volume 9, Article number: 5362 (2018)) Preferred embodiments of condensate monomers with 1 nucleotide sticky ends have short (10 base) arms and medium to high (6-12) arm number. Again, the heuristic that the interaction domains be significantly weaker than the arm domains is taken into consideration when designing the condensate monomers.

With respect to guidance on the properties of interaction domains that are Kissing Loops, note that Kissing loops have three structural features: a total length, a binding region (typically Watson Crick although for RNA it may include GU wobbles), and a flexible, disordered nonbinding region. Here, for preferred embodiments, kissing loops of up to 40 nucleotides in total length, with 4-15 nucleotide binding regions, and 0-25 nucleotide flexible regions are recommended. Kissing loops, for both DNA and RNA, have been demonstrated in both natural and artificial structures. Kissing loops for RNA are much better characterized than for DNA, RNA kissing loops have been demonstrated as interaction domains in the specific case of condensate liquids.

In the context of DNA fuels, Green et al (Green et al. “DNA Hairpins: Fuel for Autonomous DNA Devices” Biophysical Journal, Volume 91, Issue 8 p 2966-2975 Oct. 15, 2006) demonstrated kissing loops of 40 total nucleotides in length, with a binding region of 14 nucleotides and a flexible region length of 26 nucleotides. In the context of DNA circuits, Seelig et al (Seelig et al. “Enzyme-Free Nucleic Acid Logic Circuits” Science, 8 Dec. 2006, Vol 314, Issue 5805, pp. 1585-1588) demonstrated kissing loops of 27 nucleotides total length, with a binding region of 15 nucleotides and 12 nucleotides of flexible domains, distributed equally (6 nucleotides) to the 5′ and 3′ sides of the binding region. In the context of assembly DNA tetrahedrons somewhat similar to nanostars, Barth et al (Barth et al. “DNA-DNA kissing complexes as a new tool for the assembly of DNA nanostructures” Nucleic Acids Res. 2016 Jan. 14; 44(4):1502-1513. doi: 10.1093/nar/gkw014) demonstrated the use of 20 nucleotide kissing loops with a variety of flexible domains. Again, the heuristic that the interaction domains be significantly weaker than the arm domains is taken into consideration when designing the condensate monomers. In the context of successful RNA liquid condensates, Stewart et al, (Stewart et al “Modular RNA motifs for orthogonal phase separated compartments” Nature Communications volume 15, Article number: 6244 (2024)) and Fabrini et al, (Fabrini et al, “Co-transcriptional production of programmable RNA condensates and synthetic organelles” Nature Nanotechnology volume 19, pages 1665-1673 (2024)) use 9 nucleotide kissing loops of the “HIV-type” where the binding region is 6 nucleotides, and there are 3 flexible nucleotides, arranged in a pattern 5′ AA-XXXXXX-A 3′ where XXXXXX is the binding region. Other types of RNA kissing loops have been demonstrated to be effective for artificial RNA structure, in particular a 7 nucleotide “120 degree motif” demonstrated by Geary et al (Geary et al “A single-stranded architecture for cotranscriptional folding of RNA nanostructures” Science, 15 Aug. 2014, Vol 345, Issue 6198, pp. 799-804) where the binding region is 7 nucleotides long and there is no flexible linker. Thermodynamic modeling for HIV-type kissing loops is well-developed (Cao & Shen, “Structure and stability of RNA/RNA kissing complex: with application to HIV dimerization initiation signal” RNA. 2011 December; 17(12):2130-2143. doi: 10.1261/rna.026658.111). In some preferred embodiments of RNA condensates, the interaction domains are either HIV-type Kissing Loops or 120 degree type Kissing loops.

With respect to guidance for condensate monomers with blunt-end interaction domains: if blunt ends are chosen as interaction domains, then the size of the interaction is always constant (just the width of a single base-stacking interaction), and there is no opportunity for a flexible linker to be employed. However, the sequence of base pairs at the blunt end of the helices can be chosen. The strongest stacking interaction is a GC/CG interaction (−2.17 kcal in the context of normal B-DNA, see Protozanova et al. (Protozanova et al. “Stacked-Unstacked Equilibrium at the Nick Site of DNA” Journal of Molecular Biology, Volume 342, Issue 3, 17 Sep. 2004, Pages 775-785) which is used for interaction domains to assemble DNA origami into chains (see e.g. Woo & Rothemund “Programmable molecular recognition based on the geometry of DNA nanostructures” Nature Chemistry volume 3, pages 620-627 (2011)). Thus the preferred embodiment for a single layer based on blunt ends is GC, with G on the 3′ exposed end and C on the 5′ exposed end. However, other blunt end sequences have been used to assemble a large variety of 3D shapes (see e.g. Gerling et al. “Dynamic DNA devices and assemblies formed by shape-complementary, non-base pairing 3D components” Science, 27 Mar. 2015, Vol 347, Issue 6229 pp. 1446-1452) and 2D lattices (see e.g. Woo & Rothemund, “Self-assembly of two-dimensional DNA origami lattices using cation-controlled surface diffusion” Nature Communications volume 5, Article number: 4889 (2014)) Thus in some embodiments blunt end stacks other than GC may be used. The preferred embodiment for a two-layer system based on blunt ends would have a first condensate monomer having GC pairs terminating the arms, and a second condensate monomer having AT pairs terminating the arms (generating AT/TA stacks having-1.34 kcal/mol) with A on the 3′ exposed end and T on the 5′ exposed end. Woo and Rothemund show that 4 to 12 GC/CG stacking interactions give good aggregation of chains of DNA origami, and show that GC/CG stacks can give even stronger interactions (−2.63 kcal) in the context of non-B-DNA stacking geometries, which are available to condensate monomers interacting in a liquid. Preferred embodiments of condensate monomers with blunt-end based interaction domains have short (10 base) arms and high arm number (8-12 arms). The weakness and dynamic nature of stacking interactions means that they trivially satisfy the design heuristic that interaction domains be much weaker than the arm domains.

In some embodiments, other types of interaction domains are used. Any type of noncovalent interaction that is compatible with the dynamic rearrangement of monomers in a liquid condensate under appropriate thermodynamic conditions may be used. There are many small-molecule interactions which could provide appropriate interaction domains. For example Chiba et al (Chiba et al. “A supramolecular DNA self-assembly based on β-cyclodextrin-adamantane complexation as a bioorthogonal sticky end motif” Chem. Commun., 2013, 49, 6454-6456) demonstrate the assembly of DNA duplexes based on Beta-cyclodextrin and adamantane interactions, which are dynamic between 25 C and 45 C (as competition with free cyclodextrin or adamantane is readily observed). Again, the heuristic that the interaction domains be significantly weaker than the arm domains is taken into consideration when designing the condensate monomers.

### Example 10: Selection of Condensate Monomers and Experimental Conditions for Separating Biomolecular Targets

In choosing the structural features of a condensate monomer that will make it compatible with the separation of a desired biomolecular target, there is additional guidance both in terms of the structural features of the condensate monomers, and the experimental conditions under which the condensate monomers will be used.

In considering the application of nucleic acid condensates for the separation of a biomolecular target, one needs to consider (1) the properties of the biomolecular target, (2) the choice of ligand which is used to bind the target (3) the particular process used to phase separate the condensate and (4) the method of releasing the target of the condensate.

Consider first some properties of the biomolecular target. Not all biomolecular targets will be suitable for separation by nucleic acid condensates. For example, if a biomolecular target is a highly basic protein, filled with lysines and arginines so that it is highly positively charged, then it will stick very strongly to all nucleic acids nonspecifically, and the highly basic protein will stick to condensate monomers regardless of whether they have ligands that specifically bind the target. Similarly the ligand for the biomolecular target should be compatible with nucleic acid condensates. If the ligand chosen is a highly basic peptide or protein, it will stick strongly to other condensate monomers, and potentially cause condensate monomers to condense under conditions that they are not programmed to condense. Thus highly basic proteins should not be chosen for either the ligand or the target.

The sensitivity of the protein to salt and to temperature, is important. If a protein is irreversibly denatured at a certain temperature, then the condensates, and condensate separation procedure chosen work well below the denaturation temperature of the protein. If a protein aggregates under a certain salt condition, then the condensate and separation conditions are chosen to work under conditions that are different than the aggregation condition.

Similarly the sensitivity of the ligand-biomolecular target interaction to temperature are also considered. In general, a protocol like that employed in FIG. 13-17 for streptavidin, which induces condensate formation through the introduction of salt at room temperature (without the need for annealing) followed by layering of the condensate through centrifugation, and release at room temperature or below, is appropriate for ligands and biomolecular targets that are sensitive to temperatures above room temperature.

For materials made of condensate monomers, there are three regions of interest in terms of experimental conditions. There is a diffuse state, where the molecules are dispersed in an aqueous phase, a liquid state where the monomers can be readily rearranged, and a gel-like state. For the purposes of liquid layering and biomolecular separation, the two states which are most important are the diffuse state and the liquid-like state. In the diffuse state, condensate monomers can be readily mixed with samples of biomolecular targets. In the liquid-like state, condensates can be layered either singly or in combination with other condensates, and they can still be mixed with samples of biomolecular targets. In the gel state, condensates can neither be easily layered nor mixed with samples of target biomolecules.

The three states of the condensate monomers can be accessed via a variety of protocols. For example, temperature is an important variable in terms of determining the state of condensate monomers. As taught by Sato et al, (Sato et al, “Sequence-based engineering of dynamic functions of micrometer-sized DNA droplets” Science Advances, 3 Jun. 2020 Vol 6, Issue 23 DOI: 10.1126/sciadv.aba347) under a given set of salt conditions and condensate monomer concentrations, there is a temperature Td at which condensate monomers transition from all dispersed to the liquid state, and a temperate Tg at which the condensate monomers transition from the liquid state to a gel-like state. The window between Td and Tg is an important range for using condensates for layering, separation and detection. For embodiments in which biomolecular target samples are mixed with liquid condensate, this is the experimental region in which this happen. Based on structural features of the condensate, this window is readily tuned and controlled over a range of temperatures (from below room temperature to near boiling) that is suitable for essentially all possible biological experiments. For example, Sato et al (Sato et al, “Sequence-based engineering of dynamic functions of micrometer-sized DNA droplets” Science Advances, 3 Jun. 2020 Vol 6, Issue 23 DOI: 10.1126/sciadv.aba347) worked with 15 micromolar condensate monomers and 350 mM sodium. By varying sticky end length for 3-armed nanostars, they demonstrated a series of rangers (Tg<liquid state<Td) that span from 12-27° C. (4 nt) to 20-47° C. (6 nt) to 35-62° C. (8nt) to 40-60° C. (10 nt) to 53-70° C. (12 nt). By varying the arm number for 8 nt sticky ends they demonstrated a series of ranges (Tg<liquid<Td) of 35-62° C. (3 arms) to 55-68° C. (4 arms) to 60-72° C.

The literature has hundreds of examples of DNA and RNA nanostars, having different liquid phase working temperature ranges, under conditions ranging from 50 mM sodium Biffi et al. (Biffi et al, “Phase behavior and critical activated dynamics of limited-valence DNA nanostars”, PNAS 110 (39) 15633-15637) to 1 M sodium Jeon et al, (Jeon et al. “Salt-dependent properties of a coacervate-like, self-assembled DNA liquid” Soft Matter, 2018, 14, 7009-7015). All of these can serve as a starting point for the design of condensates useful in liquid layering and biomolecular separation, based on the particular needs of the separation in question. Any desired property of the liquid condensates can be significantly shifted. For example, as taught by Jeon et al. “Salt-dependent properties of a coacervate-like, self-assembled DNA liquid” Soft Matter, 2018, 14, 7009-7015) simply shifting the concentration of salt from 1M to 250 mM decrease density of typical four-arm nanostars by a factor of two, and decreases viscosity by a factor of three.

### Example 11 Choosing Experimental Conditions and Condensate Monomers for Layering

Condensates can be layered through a variety of mechanisms. In one mechanism of layering, condensates are layered via centrifugation. In one mechanism of layering, condensates are layered via thermal annealing. Mechanisms of layering can be combined, either to occur at the same time, or to occur in sequence.

An important question is, how to design sets of condensate monomers, so that when their corresponding condensates form, and are subject to a layering mechanism, the layers occur in a predictable order. Layering order is dependent on density, and so it is natural to assume that predicting and controlling layering order is dependent on accurate measurement of density. This is not the case; it is not necessary to measure the exact density of the liquid phases for a set of condensate monomers to design layered systems. Described below is a method for designing sets of nanostars, which reproducibly layer in a defined order, without the practitioner having total knowledge of the relationships between structural features and density. In brief, the method for design is based on picking a single structural feature for which a trend in density, based on varying that structural feature, is understood, and varying only that structural feature among a set of condensate monomers.

Condensate monomers have a number of structural features that affect their density, which in turn affect how their corresponding condensates will order (which condensate will end up above, and which condensate will end up below) when they are layered (via thermal annealing, centrifugation, or some other mechanism.) The features which are experimentally known to affect density include but are not limited to: arm length, strength of the interaction domains (such as sticky ends, kissing loops, or cohesive blunt ends), the number of arms. For each of these features there is a trend in density, as the feature is varied across some axis. For some of these, the trend in density has been measured, and for some of these the trend in density has not been measured. Here are some example trends which have been measured under specific experimental conditions and for which the direction (sign) of the trend is well understood:

Arm length: the longer the arms of a nanostar, the lower the density of the corresponding liquid phase.

Number of arms: the greater the number of arms, the greater the density of the corresponding liquid phase.

Average number of arms: the greater the average number of arms, the greater the density of the corresponding liquid phase.

Strength of the binding between interaction domains: the stronger the binding between interaction domains (as indicated by a lower ΔG, the free energy of association), the greater the density of the corresponding phase.

What is not known quantitatively, for general experimental conditions, is the slope of these trends (for example what the quantitative change in density is for a given change in arm length, or a given change in arm number, or a given change in binding strength).

There are also structural features, which are known to strongly affect the melting temperature of liquid condensation, and the liquid gel transition, which are hypothesized to strongly affect density. These include the flexibility of linkers between the arms (for example, the number of unpaired bases at these junctions), the flexibility of the linkers between interaction domains and the arms (for example the number of unpaired bases at these junctions), and the identity of the nucleic acid (RNA vs DNA). Because RNA interaction domains are generally stronger than the equivalent DNA interaction domains of the same sequence (with Ts converted to Us), it is predicted that RNA nanostars of the corresponding sequence to a DNA nanostar should have higher density. RNA sequences also have an extra oxygen for each nucleotide which are predicted to give RNA nanostars a higher density.

There are also experimental conditions which affect density, such as the concentration of counterions. Increasing the concentration of salt (for example sodium) increases the strength of the binding between interaction domains, which increases the density. The exact magnitude of this effect is not known for different nanostars.

Given the uncertainty in exact liquid condensate density as a function of all structural features and experimental conditions, a method for deterministically designing a layered condensate system, is to pick a single structural feature for which a clear trend in density is known (arm length, interaction domain strength, or arm number) and design a set of condensates for which only that structural feature is varied. For example, if arm length is chosen as the structural feature for a 3 layer condensate, then that structural feature is varied for a set of condensates A, B, and C, such that the (arm length of A)<(arm length of B)<(arm length of C) and the layering order, from the bottom of the tube to the top, should be A, then B, then C. For all other structural features of this set of condensates, these features should remain as constant or as constant as possible. For example, A, B, and C should all have the same number of arms. They should all have interaction strengths that are as close as possible (i.e. less than 1 kcal difference), and they should have roughly the same GC % (i.e. all in the same 5% GC window).

The question arises as to whether the variation in a particular structural feature is enough to provide a sufficient difference in density such that the particular layering method chosen can physically differentiate the condensates and provide robust ordering. Here it is best to be conservative, and pick a variation in a structural feature that is known to be sufficient for driving layering order.

With respect to giving guidance for the use of free energy (ΔG) for controlling layering, for ΔG ranging from −4 kcal/mol to −15 kcal/mol, a ΔΔG of 1 kcal/mol (between condensate monomers having the closest ΔG) is sufficient to control the layering order, and thus at least 16 layer condensate systems can be designed on this principle. We note that it is still necessary to have a matching number of orthogonal interaction domains, which is possible over the space of 6-12 nucleotide sticky ends.

In an exemplary method of a 4 layered condensate can be provided based on the design principle of varying only one structural feature in FIG. 22, where the varied structural feature is the strength of the sticky ends, based on predicted ΔG, from −11.3 (red), to 11.92 (green), to −11.98 (red), to −13.28 (green), kcal/mol). The Number of arms is constant (4) and the length of the arms is constant (20 nucleotides). Here the minimum ΔΔG is only 0.08 kcal, but as this is a single example, and ΔG predictions with different software packages easily vary by more than 0.08 kcal, a more conservative ΔΔG (e.g. 1 kcal or 0.5 kcal) should be used for engineering. Checking on the sticky end strength using different software packages, and measuring the difference in predicted sticky end strength should give a minimum ΔΔG that should be attempted. An additional example of a 3 layered condensate based on this design principle is shown in FIG. 8 Panel B at room temperature, where the ΔG of the successful liquid layers are −4.1 (green), −9.2 (yellow), and −10.6 (orange) kcal. A red condensate monomer with a ΔG of −3.5 kcal failed to condense and appears in the diffuse layer at the top of the tube. At a lower temperature the free energy of association of the weak (red) sticky end would decrease, and the red monomer would condense into a layer.

ΔΔG can be varied in units of less than 1 kcal/mol. Thus is possible that in some embodiments of layering controlled by ΔG, layering order could be controlled and more distinct layers achieved using a smaller (e.g. ΔΔG of 0.5 kcal/mol) change in free energy.

With respect to giving guidance for use of arm length for controlling layering, for a length of arms varying from 10 to 30, a change (or step) in arm length of N=3 is sufficient to control layering order, and thus at least 7 layer condensate systems can be designed on this principle (e.g. a set of arm lengths of 10, 13, 16, 19, 22, 25, and 28 or a set of arm lengths of 12, 15, 18, 21, 24, and 27. It is noted that it is still necessary to have a matching number of orthogonal interaction domains having similar ΔG to achieve this, which is possible over the space of sticky ends.

In some exemplary embodiments, of a 3 layered condensate based on the design principle of varying only one structural feature in FIG. 18d, where the varied structural feature is the arm length (from 15, to 20, to 25 nucleotides). The number of arms is constant (4), and the sticky ends were designed to have similar free energies. Here this system is layered based on centrifugation.

Arm lengths can be varied in units of N=1 if all arms of a condensate monomer are changed, and non-integral average arm lengths varying in smaller units (e.g. N=0.5) are possible if only a subset of the lengths of the arms of a nanostar are changed (e.g. a nanostar having two arms of length 10 and two arms of length 11, giving an average arm length of 10.5). Thus it is possible that in some embodiments of layering controlled by arm length, layering order could be controlled and more distinct layers achieved using a smaller (e.g. N=1) or possibly non integral (e.g. N=0.5) change in the arm length.

With respect to giving guidance for use of arm number for controlling layering, for the number of arms, from a number of arms varying from 3 to 8, a change in arm number of one is sufficient to control layering order, and thus at least 6 layer condensate systems can be designed on this principle. We note that it is still necessary to have a matching number of orthogonal interaction domains having similar ΔG to achieve this, which is possible over the space of 6-12 nucleotide sticky ends.

In some exemplary embodiment a 2 layered condensate can be provided, based on the design principle of varying only one structural feature in FIG. 7, with monomers having 4 and 6 arms.

It is possible that non-integral numbers of arms could be achieved, by for example, truncating the length of an arm in half (a nanostar with full arms of length 20 nucleotides could have half-arms of length 10 nucleotides). A nanostar with four full arms and one half-arm could thus have a number of arms of 4.5. Thus it is possible that in some embodiments of layering controlled by arm number, layering order could be controlled and more distinct layers achieved using a non-integral number of arms.

With respect to giving guidance for use of average arm number for controlling layering, for the average number of arms, from an average number of arms varying from 3 to 8, a change in average arm number of one is sufficient to control layering order, and thus 6 layer condensate systems can be designed on this principle. We note that it is still necessary to have a matching number of orthogonal interaction domains having similar ΔG to achieve this, which is possible over the space of 6-12 nucleotide sticky ends.

In an exemplary embodiment, a 3 layered condensate can be provided, based on the design principle of varying only one structural feature in FIG. 9, where the varied structural feature is the average number of arms ranges from 4 to 6 to 8 arms.

Because the average arm number for a condensate layer is controlled by mixing two or more nanostars having different fixed arm number but sharing the same interaction domains, it is possible that the average number of arms for a layer can be non-integral (e.g. an average of 4.5 arms). Thus it is possible in principle that in some embodiments of layering controlled by average arm number, layering order could be controlled, and more distinct layers achieved, using a non-integral average number of arms.

It is possible to design a layered system where more than one structural feature of the condensate monomers is changed. Because the magnitude of the trends in density as a function of a given structural feature are not generally well understood, then it is not possible to know a priori, exactly who the trends for two structural features will balance against each other, and how this will affect density, and thus layering order. For example, if a first condensate has three 10-base arms, and a second condensate has five 30-base arms, (and both condensates are the same with respect to all other structural features) it is not possible to predict which of these condensates will layer on top of the other. The first condensate would tend to be on top of the second according to the number of arms (three is less than five), but the second condensate would tend to be on top of the other based on arm length (50 is greater than 10). Thus the ordering of this pair of condensates, when layered, would have to be empirically determined, requiring a potentially large number of additional experiments. A skilled person can change more than one structural feature, and achieve predictable layering in a reliable way (without additional experiments) if the density trends for the more than one structural feature are all simultaneously respected. FIG. 23 demonstrates this approach to create a 5-layered DNA condensate. Observe that when sticky end strength and arm length are both changed (from layer 2 to 3), they are both changed in a way that increases density. Observe that when sticky end strength and valence are both changed (from layer 3 to 4, and from layer 4 to 5), they are both changed in a way that increases density, according to known trends.

The exact sequence of the condensate monomers is a structural feature of the condensate monomers. In order to prevent incorrect formation of the condensate monomers, the sequences of the condensate monomers within a layered system vary from one condensate monomer to the next. Otherwise, unless the condensate monomers were pre-made in separate tubes, the well-defined condensate monomers would not be formed. While in some embodiments it is preferred to pre-make condensate monomers in separate tubes, other preferred embodiments involve the formation of condensate monomers within the same tube where the sequences of the condensate monomers differ. In general, we say that we have not changed this structural feature (condensate monomer sequence) when the condensate monomers have roughly the same GC %—that is, they have been designed in a sequence design program with the same target GC content, for example 50% GC. The molecular weights of two different condensate monomers of the exact same size may vary slightly by a few more or less GC pairs, but the structural feature of GC content is considered to be the same.

Condensate monomers are typically labelled with fluorescent dyes so that liquid layers can be distinguished from the diffuse state, as well as from each other in a stack of layers. Fluorescent dyes are expensive, and can be sticky with each other and other molecular entities. Thus we label only a fraction (1-2%) of the condensate monomers in a nanostar. For one of the constituent strands of the condensate monomer, both nonfluorescent and fluorescent versions of the strand are synthesized. The fluorescent version typically bears the fluorescent dye on its 5′ or 3′ end. Preferred dyes include Fluorescein (FAM, for green), Takima yellow (for yellow), ATTO-550 (for orange) Texas Red (for red), and Pacific Blue (for blue). When the condensate monomers are prepared, a mixture is made with 1-2% of the fluorescent strand and 98-99% of the nonfluorescent strand.

Condensate monomers can be made in either a single-stranded format (FIG. 1A), where the interaction domains are Kissing Loops, or in a multi-stranded format (FIG. 1B), where the interaction domains are sticky ends, blunt ends, or other molecules covalently attached to the ends of arms. The question arises, when is one format (single-stranded or multi-stranded) preferred over the other. The advantages of the multi-stranded format is that it is highly modular, and strands with varying target-binding domains, and fluorophores are easily swapped without resynthesis of all of the strands comprising the condensate monomer. The multi-stranded format is ideally suited for DNA condensates, where all of the component strands are easily and cheaply chemically synthesized. Thus, where modularity is desired, and targeting domains are easily incorporated into DNA (using a DNA aptamer, a small molecule that can be attached during synthesis, or say an antibody that can be covalently attached) the multi-stranded format, rendered in DNA is preferred. In some cases the best available targeting domain is an RNA aptamer, and the condensate monomer, or at least the targeting domain part of it, is rendered in RNA. In this case the RNA aptamer may be difficult to synthesize chemically (because of length) or it might fold better in the context of cotranscriptional folding of a long RNA rather than annealing. In this case, a single-stranded architecture for the condensate monomer may be preferred.

### Example 11: Manufacturing of Functionalized Condensate Monomers that can Separate Three Different Targets at the Microscopic Level

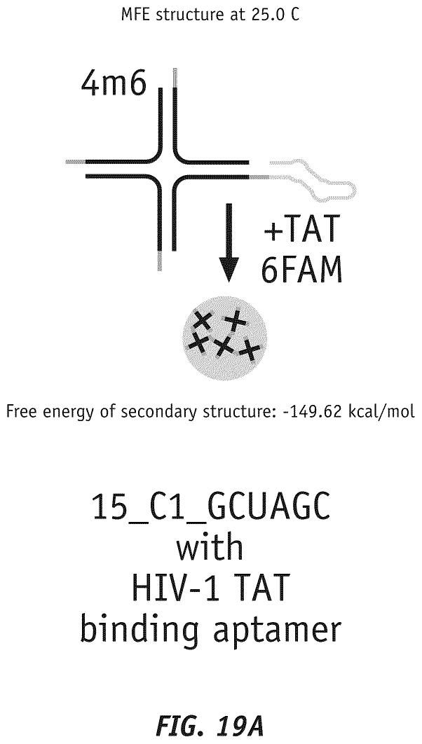

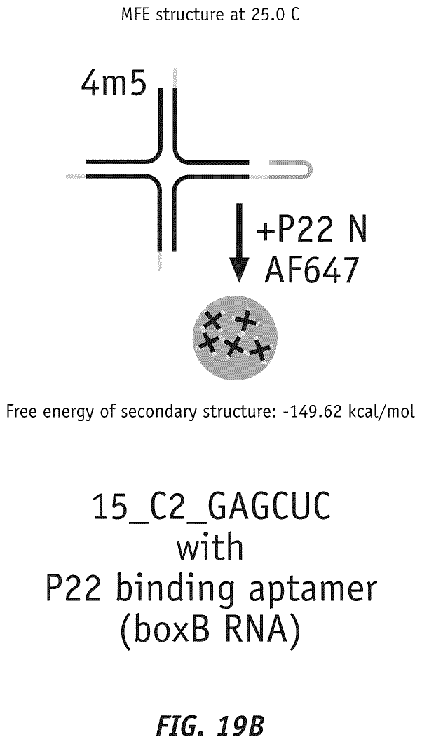

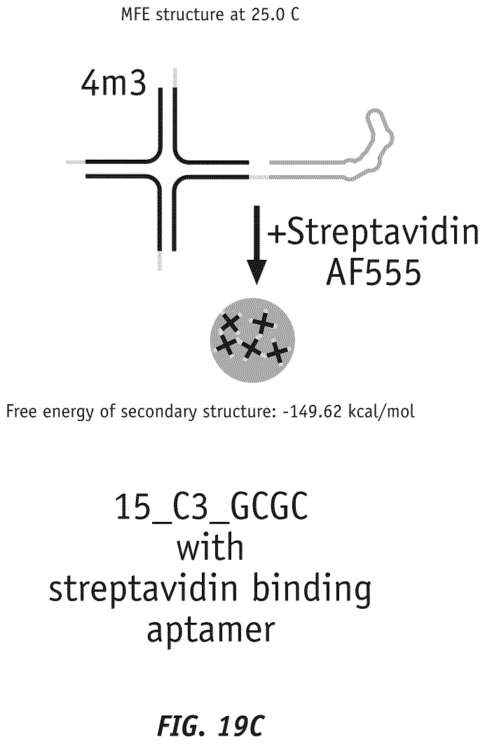

Three different functionalized monomers schematically shown in FIG. 19A to 19C formed by four armed nanostar structures were engineered to present different ligands attached to one of the duplex harm domains. The sequences are shown in FIG. 20.

FIG. 19 gives schematics for three different condensates that bind three different targets. FIG. 19A describes a condensate that has a ligand for the HIV-1 TAT peptide (labeled yellow, with FAM as the fluorophore). FIG. 19B describes a condensate that has a ligand for peptide P22 (labeled blue, with AF647 as the fluorophore). FIG. 19C describes a condensate that has a ligand for streptavidin (labeled red, with AF555 as the fluorophore. Colors are false colors and do not correspond to the normal fluorescence colors of the emitters.

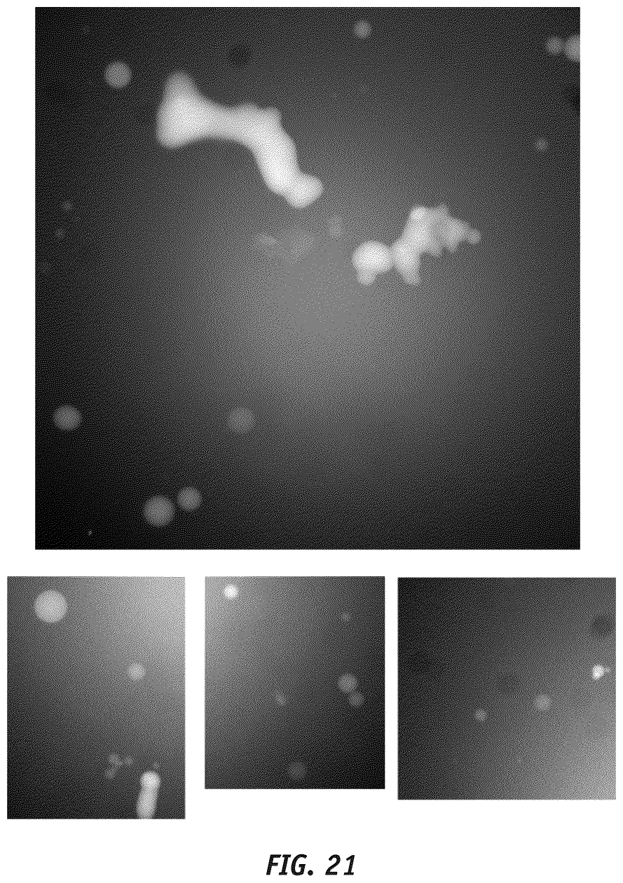

FIG. 20 gives the sequences for the RNA condensates described in FIG. 19 and experimentally imaged in FIG. 21.

FIG. 21 shows selected micrograph fields of the 3 condensates described in FIGS. 19A-C which show that at a microscopic level, three different condensates with three different ligands can compartmentalize three different targets, each one labeled with a different fluorescent color (red, yellow, or blue). The RNA condensates themselves are colorless, and would have no color without the fluorescently labeled targets. A mix of targets was applied to the condensates and the targets were spontaneously compartmentalized in the appropriate condensates.

Functionalized condensate monomers were prepared by mixing 30 ul at 5 μM motif concentration with 1.25 uM of aptamer in 40 mM HEPES, 100 mM KCl, 500 mM NaCl

The resulting mixture was subjected to thermal treatment at 70° C.: 10 min, 50° C.: 12 hr and fast cool to 20° C. Targets were added at RT and incubated at RT for ˜ 15 minutes

In particular, for all condensate sample: the following protocol was ward 6 μl condensate sample+0.5 μl of each target (350 μM-peptide, 25 μM-protein) to a final conc. ˜23.3 μM or 2.3 μM

Images were taken and are reported in FIG. 21 with the following Imaging exposure and scaling settings Cy3—100 ms—Low: 1160 High: 4705, Cy5—30 ms—Low: 3764 High: 8453 an FITC—100 ms—Low: 2464 High: 8038 and 20× objective.

All images 415 μm×415 μm showed an aptamer ligand to the four arm nanostar structure.

### Example 12: Use of Variable Arm Length to Create a 3-Layer System where all Other Structural Features are Held Constant

This example is similar to Example 4, and makes the same point that the trend in a single variable can be used for layering. This shows that a difference of 5 nucleotides in arm length is sufficient to provide density contrast and reproducibly determine the ordering of liquid layers.

We have synthesized a series of three immiscible DNA condensates (FIG. 18d) whose monomers' difference in size (arm length) creates large enough differences in density that they can be separated by low-speed centrifugation. This validates the design of a 3 layered condensate based on the design principle of varying only one structural feature, where the varied structural feature is the arm length (from 15, to 20, to 25 nucleotides). The number of arms is constant (4), and the sticky ends were designed to have similar free energies. Here this system is layered based on centrifugation.

Detailed protocol for FIG. 18d: All 12 strands for the three nanostars were mixed together at a concentration of 100 μM each in 10 mM TE, 250 mM NaCl. Sticky ends have similar free energies. 1-2% FAM, Yakima Yellow, and Texas Red modified strands were added for the 15, 20, and 25 arm length nanostars. The mixture was heated at 90° C. for 5 minutes and then annealed from 90° C. to 20° C. at a rate of 1° C./min. The mixture was next centrifuged at 15,000×g for 1 hour. Sequence details are:

For the 15-nt arm length:
SEQ ID NO 1:
GTAGTCTACAGTGCCAACTGGGCAGAATTCCCAGCTAGC;
SEQ ID NO 2:
GGGAATTCTGCCCAGAACGTCACCAGAAGCACAGCTAGC;
SEQ ID NO 3:
GTGCTTCTGGTGACGAAGACGGAATCTCCGTCAGCTAGC;
SEQ ID NO 4:
GACGGAGATTCCGTCAAGGCACTGTAGACTACAGCTAGC
For the 20-nt arm length:
SEQ ID NO 5:
CTACTATGGCGGGTGATAAAAACGGGAAGAGCATGCCCATCCACGATCG;
SEQ ID NO 6:
GGATGGGCATGCTCTTCCCGAACTCAACTGCCTGGTGATACGACGATCG;
SEQ ID NO 7:
CGTATCACCAGGCAGTTGAGAACATGCGAGGGTCCAATACCGACGATCG;
SEQ ID NO 8:
CGGTATTGGACCCTCGCATGAATTTATCACCCGCCATAGTAGACGATCG
For the 25-nt arm length:
SEQ ID NO 9:
CGCTACAATACAGTTACAAGAATGCAACGCTTGATGTATGCACGTATGTT
GCACACGTG;
SEQ ID NO 10:
GCAACATACGTGCATACATCAAGCGAACATATCTCATATTCGTGCCACTA
TGACACGTG;
SEQ ID NO 11:
CATAGTGGCACGAATATGAGATATGAACAGTAGGGCAGCAAAGACTACGG
TGACACGTG;
SEQ ID NO 12:
CACCGTAGTCTTTGCTGCCCTACTGAAGCATTCTTGTAACTGTATTGTAG
CGACACGTG

In summary described herein are. functionalized nucleic acid condensate monomers and related functionalized set of nucleic acid condensate monomers, functionalized nucleic acid condensate, as well as related composition methods and systems of manufacturing and related for separation, purification and/or detection of one or more biomolecular targets.

The examples set forth above are provided to give those of ordinary skill in the art a complete disclosure and description of how to make and use the embodiments of the materials, compositions, systems and methods of the disclosure, and are not intended to limit the scope of what the inventors regard as their disclosure. Those skilled in the art will recognize how to adapt the features of the exemplified methods and systems based on the target compound removing agents, nucleic acid removing agents, solid matrices, and devices according to various embodiments and scope of the claims.

All patents and publications mentioned in the specification are indicative of the levels of skill of those skilled in the art to which the disclosure pertains.

The entire disclosure of each document cited (including webpages patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified can be employed in the practice of the disclosure without resort to undue experimentation. All art-known functional equivalents, of any such methods, device elements, and materials are intended to be included in this disclosure.

Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein can be excluded from a claim of this disclosure. The disclosure illustratively described herein suitably can be practiced in the absence of any element or elements, limitation or limitations, which is not specifically disclosed herein.

“Optional” or “optionally” means that the subsequently described circumstance may or may not occur, so that the description includes instances where the circumstance occurs and instances where it does not according to the guidance provided in the present disclosure. For example, the phrase “optionally substituted” means that a non-hydrogen substituent may or may not be present on a given atom, and, thus, the description includes structures wherein a non-hydrogen substituent is present and structures wherein a non-hydrogen substituent is not present. It will be appreciated that the phrase “optionally substituted” is used interchangeably with the phrase “substituted or unsubstituted.” Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned can be identified in view of the desired features of the compound in view of the present disclosure, and in view of the features that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

A number of embodiments of the disclosure have been described. The specific embodiments provided herein are examples of useful embodiments of the disclosure and it will be apparent to one skilled in the art that the disclosure can be carried out using a large number of variations of the devices, device components, methods steps set forth in the present description. As will be obvious to one of skill in the art, methods and devices useful for the present methods can include a large number of optional composition and processing elements and steps.

In particular, it will be understood that various modifications may be made without departing from the spirit and scope of the present disclosure. Accordingly, other embodiments are within the scope of the following claims.

### REFERENCES

1. Biffi, S., et al., Phase behavior and critical activated dynamics of limited-valence DNA nanostars. Proceedings of the National Academy of Sciences, 2013. 110(39): p. 15633-15637.
2. Alberti, S., Phase separation in biology. Current Biology, 2017. 27(20): p. R1097-R1102.
3. Sato, Y., T. Sakamoto, and M. Takinoue, Sequence design-based control of DNA droplets formed from phase separation of DNA nanostructures. arXiv preprint arXiv: 1907.10252, 2019.
4. Jeon, B.-j., et al., Salt-dependent properties of a coacervate-like, self-assembled DNA liquid. Soft Matter, 2018. 14(34): p. 7009-7015.
5. Rolando, J. C., et al., Real-time, digital LAMP with commercial microfluidic chips reveals the interplay of efficiency, speed, and background amplification as a function of reaction temperature and time. Analytical chemistry, 2018. 91(1): p. 1034-1042.
6. Niu, X., et al., Biomolecular condensates: Formation mechanisms, biological functions, and therapeutic targets. MedComm, 2023. 4(2): p. e223.
7. Forman-Kay, J. D., et al., What are the distinguishing features and size requirements of biomolecular condensates and their implications for RNA-containing condensates? RNA, 2022. 28(1): p. 36-47.
8. Nguyen, D. T. and O. A. Saleh, Tuning phase and aging of DNA hydrogels through molecular design. Soft Matter, 2017. 13(32): p. 5421-5427.
9. Wang, J., X. Pan, and X. Liang, Assessment for melting temperature measurement of nucleic acid by HRM. Journal of analytical methods in chemistry, 2016. 2016(1): p. 5318935.
10. Glaser, M., et al., The art of designing DNA nanostructures with CAD software. Molecules, 2021. 26(8): p. 2287.
11. Elonen, A., et al., DNAforge: a design tool for nucleic acid wireframe nanostructures. Nucleic Acids Research, 2024: p. gkae367.
12. Poppleton, E., et al., Design, optimization and analysis of large DNA and RNA nanostructures through interactive visualization, editing and molecular simulation. Nucleic acids research, 2020. 48(12): p. e72-e72.
13. Woo, S. and P. W. Rothemund, Programmable molecular recognition based on the geometry of DNA nanostructures. Nature chemistry, 2011. 3(8): p. 620-627.
14. Seeger, M. A., et al., Design, construction, and characterization of a second-generation DARPin library with reduced hydrophobicity. Protein science, 2013. 22(9): p. 1239-1257.
15. Mast, F. D., et al., Highly synergistic combinations of nanobodies that target SARS-CoV-2 and are resistant to escape. Elife, 2021. 10: p. e73027.
16. Pendergrast, P. S., et al., Nucleic acid aptamers for target validation and therapeutic applications. Journal of biomolecular techniques: JBT, 2005. 16(3): p. 224.
17. Wang, F., et al., Nucleic acids and their analogues for biomedical applications. Biosensors, 2022. 12(2): p. 93.
18. Kick, A., M. Bönsch, and M. Mertig, EGNAS: an exhaustive DNA sequence design algorithm. BMC bioinformatics, 2012. 13: p. 1-17.
19. Collette, D., D. Dunlap, and L. Finzi, Macromolecular Crowding and DNA: Bridging the Gap between In Vitro and In Vivo. International Journal of Molecular Sciences, 2023. 24(24): p. 17502.
20. Zadeh, J. N., et al., NUPACK: Analysis and design of nucleic acid systems. Journal of computational chemistry, 2011. 32(1): p. 170-173.
21. Dirks, R. M., et al., Thermodynamic analysis of interacting nucleic acid strands. SIAM review, 2007. 49(1): p. 65-88.
22. Dirks, R. M. and N. A. Pierce, A partition function algorithm for nucleic acid secondary structure including pseudoknots. Journal of computational chemistry, 2003. 24(13): p. 1664-1677.
23. Dirks, R. M. and N. A. Pierce, An algorithm for computing nucleic acid base-pairing probabilities including pseudoknots. Journal of computational chemistry, 2004. 25(10): p. 1295-1304.
24. Zadeh, J. N., B. R. Wolfe, and N. A. Pierce, Nucleic acid sequence design via efficient ensemble defect optimization. Journal of computational chemistry, 2011. 32(3): p. 439-452.
25. Zuker, M., Mfold web server for nucleic acid folding and hybridization prediction. Nucleic acids research, 2003. 31(13): p. 3406-3415.
26. Waugh, A., et al., RNAML: a standard syntax for exchanging RNA information. Rna, 2002. 8(6): p. 707-717.
27. Zuker, M. and A. B. JACOBSON, Using reliability information to annotate RNA secondary structures. Rna, 1998. 4(6): p. 669-679.
28. Markham, N. R. and M. Zuker, UNAFold: software for nucleic acid folding and hybridization. Bioinformatics: structure, function and applications, 2008: p. 3-31.
29. Zuker, M., D. Mathews, and D. Turner, Algorithms and thermodynamics for RNA secondary structure prediction: A practical guide+ In: Barciszewski J, Clark BFC, eds+ RNA biochemistry and biotechnology. NATO ASI Series+ Dordrecht, The Netherlands: Kluwer Academic Publishers, 1999: p. 11-43.
30. Zuker, M., Prediction of RNA secondary structure by energy minimization. In “Computer Analysis of Sequence Data, Part II” (AM Griffin and HG Griffin, Eds.), Vol. 25. 1994, CRC Press, Totowa, NJ.
31. Jaeger, J. A., D. H. Turner, and M. Zuker, Predicting optimal and suboptimal secondary structure for RNA. 1990.
32. Hermanson, G. T., Bioconjugate techniques. 2013: Academic press.
33. Gyawali, B., K. Ramakrishna, and A. S. Dhamoon, Sepsis: The evolution in definition, pathophysiology, and management. SAGE open medicine, 2019. 7: p. 2050312119835043.
34. Torio, C. M. and B. J. Moore, National inpatient hospital costs: the most expensive conditions by payer, 2013. 2016.
35. Reimer, L. G., M. L. Wilson, and M. P. Weinstein, Update on detection of bacteremia and fungemia. Clinical microbiology reviews, 1997. 10(3): p. 444-465.
36. Brangwynne, C. P., et al., Germline P granules are liquid droplets that localize by controlled dissolution/condensation. Science, 2009. 324(5935): p. 1729-1732.
37. Feric, M., et al., Coexisting liquid phases underlie nucleolar subcompartments. Cell, 2016. 165(7): p. 1686-1697.
38. Itakura, A. K., R. A. Futia, and D. F. Jarosz, It pays to be in phase. Biochemistry, 2018. 57(17): p. 2520-2529.
39. Reinkemeier, C. D., G. E. Girona, and E. A. Lemke, Designer membraneless organelles enable codon reassignment of selected mRNAs in eukaryotes. Science, 2019. 363(6434): p. eaaw2644.
40. Woodruff, J. B., et al., The centrosome is a selective condensate that nucleates microtubules by concentrating tubulin. Cell, 2017. 169(6): p. 1066-1077. e10.
41. Tuerk, C. and L. Gold, Systematic evolution of ligands by exponential enrichment: RNA ligands to bacteriophage T4 DNA polymerase. science, 1990. 249 (4968): p. 505-510.
42. Ellington, A. D. and J. W. Szostak, In vitro selection of RNA molecules that bind specific ligands. nature, 1990. 346(6287): p. 818-822.
43. Marton, S., et al., Isolation of an aptamer that binds specifically to E. coli. PloS one, 2016. 11(4): p. e0153637.
44. Lim JaeWoo, L. J., et al., Peptidoglycan binding protein (PGBP)-modified magnetic nanobeads for efficient magnetic capturing of Staphylococcus aureus associated with sepsis in blood. 2019.
45. Schoepp, N. G., et al., Rapid pathogen-specific phenotypic antibiotic susceptibility testing using digital LAMP quantification in clinical samples. Science translational medicine, 2017. 9(410): p. eaal3693.
46. Ma, Y.-D., et al., Digital quantification of DNA via isothermal amplification on a self-driven microfluidic chip featuring hydrophilic film-coated polydimethylsiloxane. Biosensors and Bioelectronics, 2018. 99: p. 547-554.
47. Ma, Y.-D., et al., A microfluidic chip capable of generating and trapping emulsion droplets for digital loop-mediated isothermal amplification analysis. Lab on a Chip, 2018. 18(2): p. 296-303.
48. Schuler, F., et al., Digital droplet LAMP as a microfluidic app on standard laboratory devices. Analytical Methods, 2016. 8(13): p. 2750-2755.
49. Song, M. Y., et al., Broadly reactive aptamers targeting bacteria belonging to different genera using a sequential toggle cell-SELEX. Scientific reports, 2017. 7(1): p. 43641.
50. Shin, H.-S., et al., Detection of Gram-negative bacterial outer membrane vesicles using DNA aptamers. Scientific Reports, 2019. 9(1): p. 13167.
51. Ferreira, I. M., et al., Selection of peptidoglycan-specific aptamers for bacterial cells identification. Applied biochemistry and biotechnology, 2014. 174: p. 2548-2556.
52. Graziani, A. C., et al., High efficiency binding aptamers for a wide range of bacterial sepsis agents. Journal of microbiology and biotechnology, 2017. 27(4): p. 838-843.
53. Norouzi, N., H. C. Bhakta, and W. H. Grover, Sorting cells by their density. PloS one, 2017. 12(7): p. e0180520.
54. Chang, Y.-C., et al., Rapid single cell detection of Staphylococcus aureus by aptamer-conjugated gold nanoparticles. Scientific reports, 2013. 3(1): p. 1863.
55. Scheideler, O. J., et al., Recapitulating complex biological signaling environments using a multiplexed, DNA-patterning approach. Science advances, 2020. 6(12): p. eaay5696.
56. Zhang, D. Y. and G. Seelig, Dynamic DNA nanotechnology using strand-displacement reactions. Nature chemistry, 2011. 3(2): p. 103-113.
57. Song, W., et al., Imaging RNA polymerase III transcription using a photostable RNA-fluorophore complex. Nature chemical biology, 2017. 13(11): p. 1187-1194.
58. Cross, A. S., et al., A hospital-wide outbreak of septicemia due to a few strains of Staphylococcus aureus. American Journal of Clinical Pathology, 1983. 79(5): p. 598-603.
59. Vihta, K.-D., et al., Trends over time in Escherichia coli bloodstream infections, urinary tract infections, and antibiotic susceptibilities in Oxfordshire, U K, 1998-2016: a study of electronic health records. The Lancet Infectious Diseases, 2018. 18(10): p. 1138-1149.
60. Lee, H.-J., et al., A sensitive method to detect Escherichia coli based on immunomagnetic separation and real-time PCR amplification of aptamers. Biosensors and Bioelectronics, 2009. 24(12): p. 3550-3555.
61. Keeble, A. H., et al., Evolving accelerated amidation by SpyTag/SpyCatcher to analyze membrane dynamics. Angewandte Chemie International Edition, 2017. 56(52): p. 16521-16525.
62. Scaffold, P. D. O., Tug-of-War in Motor Protein Ensembles Revealed with. Mol. Plant Microbe Interact, 2008. 21: p. 7.
63. Lovendahl, K. N., A. N. Hayward, and W. R. Gordon, Sequence-directed covalent protein-DNA linkages in a single step using HUH-tags. Journal of the American Chemical Society, 2017. 139(20): p. 7030-7035.

1-70. (canceled)

71. A functionalized nucleic acid condensate monomer, configured to specifically bind a biomolecular target, and to form in an aqueous solution and in combination with a same or different nucleic acid condensate monomers a functionalized nucleic acid condensate via liquid-liquid phase separation (LLPS) at condensing thermodynamic conditions,
the functionalized nucleic acid condensate monomer comprising a single stranded or a multistranded nucleic acid complex having up to twelve double-stranded arms,
wherein at least one double-stranded arm of the up to twelve double-stranded arms, is configured to interact with another double stranded arm of another functionalized nucleic acid condensate monomer, to form the functionalized nucleic acid condensate, and
wherein double-stranded arms of the up to twelve double-stranded arms is functionalized with a ligand configured to specifically bind the biomolecular target in the aqueous solution.

72. The functionalized nucleic acid condensate monomer of claim 71, wherein the functionalized nucleic acid condensate monomer is a multistranded nucleic acid complex.

73. The functionalized nucleic acid condensate monomer of claim 71, wherein the single stranded or multistranded nucleic acid complex is formed by one or more nucleic acid strands selected from DNA strands, RNA strands, PNA strands and/or LNA strands.

74. The functionalized nucleic acid condensate monomer of claim 71, wherein an interaction between the at least one double-stranded arm and the another double stranded arm is performed through complementary overlaps.

75. The functionalized nucleic acid condensate monomer of claim 74, wherein the complementary overlaps are sticky ends of the at least one double stranded arm and the another double stranded arm.

76. The functionalized nucleic acid condensate monomer of claim 71, wherein the single stranded or a multistranded nucleic acid complex has 4 to 6 double stranded arms.

77. The functionalized nucleic acid condensate monomer of claim 71, wherein the up to twelve double-stranded arms have a length ranging from 15 to 25 nt.

78. The functionalized nucleic acid condensate monomer of claim 71, wherein the n the single stranded or a multistranded nucleic acid complex is a multi-armed nucleic acid nanostar structure.

79. The functionalized nucleic acid condensate monomer of claim 78, wherein arms of the nanostar structure are connected by unpaired bases.

80. The functionalized nucleic acid condensate monomer of claim 71, wherein the ligand is presented on a terminus of a duplex arm segment.

81. The functionalized nucleic acid condensate monomer of claim 71, wherein the ligand is a small molecule, an aptamers, an antibody or a fragment thereof, a nanobody, or a DARPin.

82. The functionalized nucleic acid condensate monomer of claim 71, wherein the biomolecular target is one of a protein, a nucleic acid or a cell.

83. The functionalized nucleic acid condensate monomer of claim 82, wherein the biomolecular target is DNA or RNA.

84. A functionalized set of nucleic acid condensate monomers, configured to specifically bind a biomolecular target, and to form, in aqueous solvent and under condensing thermodynamic conditions, a functionalized nucleic acid condensate having a distinct nucleic acid condensate density via liquid-liquid phase separation (LLPS),
the functionalized set of nucleic acid condensate monomers comprising one or more functionalized nucleic acid monomers of claim 71,
each functionalized nucleic acid monomer
configured to form a nucleic acid condensate at the condensing thermodynamic conditions through intermolecular interactions of interaction domains of another, same or different nucleic acid condensate monomer of the set of nucleic acid condensate monomers, and
presenting at least one ligand configured to specifically bind the biomolecular target.

85. The functionalized set of nucleic acid condensate monomers of claim 84, wherein the interaction domains of nucleic acid condensate monomers of the set of nucleic acid condensate monomers are sticky overhangs.

86. The functionalized set of nucleic acid condensate monomers of claim 84, wherein the one or more functionalized nucleic acid monomers are formed by a same functionalized nucleic acid monomer presenting self-binding interaction domains, preferably self-binding overhangs.

87. The functionalized set of nucleic acid condensate monomers of claim 84, wherein the one or more functionalized nucleic acid monomers have a same valency.

88. The functionalized set of nucleic acid condensate monomers of claim 84, wherein the valency is selected from 4 to 6.

89. The functionalized set of nucleic acid condensate monomers of claim 84, wherein the one or more functionalized monomers comprises a functionalized nucleic acid nanostructure.

90. The functionalized set of nucleic acid condensate monomers of claim 84, wherein the biomolecular target comprises a plurality of biomolecular target and the functionalized set of nucleic acid condensate monomers comprises a plurality of functionalized nucleic acid condensate monomers each presenting a ligand for a biomolecular target of the plurality of biomolecular targets.

91. A functionalized nucleic acid condensate having a distinct functionalized nucleic acid condensate density and configured to bind a biomolecular target, the functionalized nucleic acid condensate comprising
a functionalized set of nucleic acid condensate monomers of claim 84 configured to form in aqueous solvent and under the condensing thermodynamic conditions, a nucleic acid condensate having the distinct functionalized nucleic acid condensate density, via liquid-liquid phase separation (LLPS),
the functionalized nucleic acid condensate comprising ligand configured to bind the biomolecular target and presented on the nanostructure for binding with the target biomolecular target when present.

92. The functionalized set of nucleic acid condensate of claim 91, wherein all condensate monomers of the set of functionalized nucleic acid condensate monomers have a same valency.

93. A functionalized layered nucleic acid condensate layered structure comprising:
at least one functionalized nucleic acid condensate layer formed by a functionalized nucleic acid condensate of claim 91, each condensate layer having a distinct condensate density and comprising a distinct interaction domains and a distinct ligand specific for distinct one or more biomolecular targets,
wherein at condensing thermodynamic conditions the at least one functionalized nucleic acid condensate layer is arranged in the structure, in order of increasing density, with the densest layer positioned opposite the least dense layer within the structure.

94. The functionalized layered nucleic acid condensate structure of claim 93, further comprising a diffuse layer wherein the diffuse layer is the least dense layer in the structure.

95. The functionalized layered nucleic acid condensate structure of claim 93, wherein functionalized nucleic acid condensate layer structure is formed by a single functionalized condensate layer.

96. The functionalized layered nucleic acid condensate structure of claim 93, wherein functionalized nucleic acid layered condensate structure is formed by a plurality of functionalized condensate layers.

97. The functionalized layered nucleic acid condensate structure of claim 93, wherein each functionalized nucleic acid condensate layer is formed by functionalized nucleic acid condensate monomers presenting interaction domains orthogonal to interaction domains of functionalized condensate monomer of another functionalized nucleic acid condensate layer of the layered condensate structure.

98. The functionalized layered nucleic acid condensate structure of claim 93, wherein each functionalized nucleic acid condensate layer comprises ligand specific for a single distinct biomolecular target.

99. The functionalized layered nucleic acid condensate structure of claim 93, wherein each functionalized nucleic acid condensate layer comprises ligand specific for a plurality of distinct biomolecular targets.

100. A method to separate a biomolecular target from a mixture in which the biomolecular target is comprised together with additional compounds, the method comprising
providing the set of functionalized nucleic acid condensate monomers of claim 84 functionalized with a ligand specific for the biomolecular target,
contacting the set of functionalized nucleic acid condensate monomers with the mixture to allow binding of the biomolecular target with the ligand, and following the contacting with the mixture, inducing condensation of the set of functionalized nucleic acid condensate monomers to form a functionalized nucleic acid condensate,
layering of the functionalized nucleic acid condensate in nucleic acid condensate layer within a functionalized layered nucleic acid condensate structure;
releasing the biomolecular target from the ligand of the se of functionalized nucleic acid monomers to obtain the release of the biomolecular target from the nucleic acid condensate; and optionally
separating the released biomolecular target from the nucleic acid condensate layer.

101. A system to separate a biomolecular target from a mixture in which the biomolecular target is comprised together with additional compounds, the system comprising
at least one set of functionalized nucleic acid condensate monomers of claim 84, functionalized with a ligand specific for the biomolecular target, and
one or more release agents or devices capable of releasing the biomolecular target from the functionalized nucleic acid condensate monomer.

102. A method to provide a functionalized nucleic acid condensate, the method comprising
providing a set of functionalized nucleic acid condensate monomers of claim 84 configured to form a nucleic acid condensate having a distinctive density; and
inducing condensation of the set of functionalized condensate monomer to form a functionalized nucleic acid condensate having the distinctive density.

103. A system to provide a functionalized nucleic acid condensate, the system comprising
a set of functionalized condensate monomers of claim 84 configured to form a nucleic acid condensate having a same density in combination with condensation agents.


SEQUENCE LISTING (34 sequences)

SEQ ID NO: 1 - DNA, 39 bp, synthetic construct
  source 1..39: mol_type = other DNA
gtagtctacagtgccaactgggcagaattcccagctagc

SEQ ID NO: 2 - DNA, 39 bp, synthetic construct
  source 1..39: mol_type = other DNA
gggaattctgcccagaacgtcaccagaagcacagctagc

SEQ ID NO: 3 - DNA, 39 bp, synthetic construct
  source 1..39: mol_type = other DNA
gtgcttctggtgacgaagacggaatctccgtcagctagc

SEQ ID NO: 4 - DNA, 39 bp, synthetic construct
  source 1..39: mol_type = other DNA
gacggagattccgtcaaggcactgtagactacagctagc

SEQ ID NO: 5 - DNA, 49 bp, synthetic construct
  source 1..49: mol_type = other DNA
ctactatggcgggtgataaaaacgggaagagcatgcccatccacgatcg

SEQ ID NO: 6 - DNA, 49 bp, synthetic construct
  source 1..49: mol_type = other DNA
ggatgggcatgctcttcccgaactcaactgcctggtgatacgacgatcg

SEQ ID NO: 7 - DNA, 49 bp, synthetic construct
  source 1..49: mol_type = other DNA
cgtatcaccaggcagttgagaacatgcgagggtccaataccgacgatcg

SEQ ID NO: 8 - DNA, 49 bp, synthetic construct
  source 1..49: mol_type = other DNA
cggtattggaccctcgcatgaatttatcacccgccatagtagacgatcg

SEQ ID NO: 9 - DNA, 59 bp, synthetic construct
  source 1..59: mol_type = other DNA
cgctacaatacagttacaagaatgcaacgcttgatgtatgcacgtatgttgcacacgtg

SEQ ID NO: 10 - DNA, 59 bp, synthetic construct
  source 1..59: mol_type = other DNA
gcaacatacgtgcatacatcaagcgaacatatctcatattcgtgccactatgacacgtg

SEQ ID NO: 11 - DNA, 59 bp, synthetic construct
  source 1..59: mol_type = other DNA
catagtggcacgaatatgagatatgaacagtagggcagcaaagactacggtgacacgtg

SEQ ID NO: 12 - DNA, 59 bp, synthetic construct
  source 1..59: mol_type = other DNA
caccgtagtctttgctgccctactgaagcattcttgtaactgtattgtagcgacacgtg

SEQ ID NO: 13 - DNA, 33 bp, synthetic construct
  source 1..33: mol_type = other DNA
gcattgaccgctgtgtgacgcaacactcaatgc

SEQ ID NO: 14 - DNA, 58 bp, synthetic construct
  source 1..58: mol_type = other DNA
gcattgaccgctgtgtgacgcaacactcaatgcttaggcatgttaagtagacatatcg

SEQ ID NO: 15 - DNA, 38 bp, synthetic construct
  source 1..38: mol_type = other DNA
cctaagcattgagtgttgcgtcacacagcggtcaatgc

SEQ ID NO: 16 - RNA, 37 nt, synthetic construct
  source 1..37: mol_type = other RNA
gcgcatccacggcgtgtcgcaagacgccgacgcgtcg

SEQ ID NO: 17 - RNA, 37 nt, synthetic construct
  source 1..37: mol_type = other RNA
gcgcagtcgggatataccggaagcgacacgccgtgga

SEQ ID NO: 18 - RNA, 37 nt, synthetic construct
  source 1..37: mol_type = other RNA
gcgcaagtccacagtggcgcaaccggtatatcccgac

SEQ ID NO: 19 - RNA, 37 nt, synthetic construct
  source 1..37: mol_type = other RNA
gcgcacgacgcgtcggcgtcaagcgccactgtggact

SEQ ID NO: 20 - RNA, 99 nt, synthetic construct
  source 1..99: mol_type = other RNA
gatgcggccgccgaccagaatcatgcaagtgcgtaagatagtcgcgggtcggcggccgcatcgcgcatccacggcgtgtcgcaagacgccgacgcgtcg

SEQ ID NO: 21 - RNA, 39 nt, synthetic construct
  source 1..39: mol_type = other RNA
gagctcagagtctctggtcgccaagcttatcggacactg

SEQ ID NO: 22 - RNA, 39 nt, synthetic construct
  source 1..39: mol_type = other RNA
gagctcaagtaggcacatcagcaaggcgaccagagactc

SEQ ID NO: 23 - RNA, 39 nt, synthetic construct
  source 1..39: mol_type = other RNA
gagctcatgcgtggagacaacgaagctgatgtgcctact

SEQ ID NO: 24 - RNA, 39 nt, synthetic construct
  source 1..39: mol_type = other RNA
gagctcacagtgtccgataagcaacgttgtctccacgca

SEQ ID NO: 25 - RNA, 60 nt, synthetic construct
  source 1..60: mol_type = other RNA
ggtgcgctgacaaagcgcgccgagctcagagtctctggtcgccaagcttatcggacactg

SEQ ID NO: 26 - RNA, 39 nt, synthetic construct
  source 1..39: mol_type = other RNA
gctagcaggtcagtgtgaacgcaagcgagagcgctgccc

SEQ ID NO: 27 - RNA, 39 nt, synthetic construct
  source 1..39: mol_type = other RNA
gctagcagggcagcgctctcgcaagcgtatcactacacc

SEQ ID NO: 28 - RNA, 39 nt, synthetic construct
  source 1..39: mol_type = other RNA
gctagcaggtgtagtgatacgcaagcgatgaacgtaggg

SEQ ID NO: 29 - RNA, 39 nt, synthetic construct
  source 1..39: mol_type = other RNA
gctagcaccctacgttcatcgcaagcgttcacactgacc

SEQ ID NO: 30 - RNA, 71 nt, synthetic construct
  source 1..71: mol_type = other RNA
ggagcttgatcccggaaacggtcgatcgctccgctagcaggtcagtgtgaacgcaagcgagagcgctgccc

SEQ ID NO: 31-34 - skipped (4 entries)